(19) United States
(12) Patent Application Publication
(10) Pub. No.: US 2026/0325010 A1
(43) Pub. Date: Oct. 22, 2026

(54) MONITORING AND MANAGEMENT OF A CONTINUOUS ANALYTE MONITORING SYSTEM

(71) Applicant: Biolinq Incorporated, San Diego, CA (US)

(72) Inventors: Keith NOGUEIRA, San Diego, CA (US); Richard Chien YANG, Carlsbad, CA (US); Cecilia LANG-REE, San Francisco, CA (US); Alexis SCHOENBERG, Oakland, CA (US); Hallie Fei SMITH, San Diego, CA (US); Bora HWANG, Los Angeles, CA (US); Rupert SKINNER, Heathfield (GB); Albert CAI, San Diego, CA (US)

(21) Appl. No.: 19/536,158

(22) Filed: Feb. 10, 2026

Related U.S. Application Data
(60) Provisional application No. 63/756,773, filed on Feb. 10, 2025.
(60) Provisional application No. 63/826,339, filed on Jun. 18, 2025.

(51) Int. Cl. A61B 5/11; A61B 5/00; A61B 5/145; G01J 1/42; G16H 20/30

(52) U.S. Cl. CPC A61B 5/1118; A61B 5/14503; A61B 5/14532; A61B 5/4809; A61B 5/6801; G01J 1/4204; G16H 20/30

(57) ABSTRACT

Aspects are directed to a wearable analyte monitoring device including a microneedle array configured to detect an analyte in interstitial fluid of a user. The wearable analyte monitoring device also includes sensors to determine a state of a user, including for example periods of rest and periods of activity, using one or more signals from the sensors. The sensors include an ambient light sensor, a kinetic sensor, and a clock.

### CROSS REFERENCE TO RELATED APPLICATIONS

This application claims priority to U.S. Provisional Patent Application No. 63/756,773, filed Feb. 10, 2025, and to U.S. Provisional Patent Application No. 63/826,339, filed Jun. 18, 2025, the content of each of which is hereby incorporated by reference herein in its entirety.

### TECHNICAL FIELD

This invention relates generally to the field of analyte monitoring, such as continuous glucose monitoring.

### BACKGROUND

Diabetes is a chronic disease in which the body does not produce or properly utilize insulin, a hormone that regulates blood glucose. Insulin may be administered to a diabetic patient to help regulate blood glucose levels, though blood glucose levels must nevertheless be carefully monitored to help ensure that timing and dosage are appropriate. Without proper management of their condition, diabetic patients may suffer from a variety of complications resulting from hyperglycemia (high blood sugar levels) or hypoglycemia (low blood sugar levels).

Blood glucose monitors help diabetic patients manage their condition by measuring blood glucose levels from a sample of blood. For example, a diabetic patient may obtain a blood sample through a fingerstick sampling mechanism, transfer the blood sample to a test strip with suitable reagent(s) that react with the blood sample, and use a blood glucose monitor to analyze the test strip to measure glucose level in that blood sample. However, a patient using this process can typically only measure his or her glucose levels at discrete instances in time, which may fail to capture a hyperglycemia or hypoglycemia condition in a timely manner. Yet a more recent variety of glucose monitor is a continuous glucose monitor (CGM) device, which includes implantable transdermal electrochemical sensors that are used to continuously detect and quantify blood glucose levels by proxy measurement of glucose levels in the subcutaneous interstitial fluid. However, conventional CGM devices also have weaknesses including tissue trauma from insertion and signal latency (e.g., due to the time required for the glucose analyte to diffuse from capillary sources to the sensor). These weaknesses also lead to a number of drawbacks, such as pain experienced by the patient when electrochemical sensors are inserted, and limited accuracy in glucose measurements, particularly when blood glucose levels are changing rapidly. Accordingly, there is a need for a new and improved analyte monitoring system.

### SUMMARY

The present disclosure generally relates to detecting a state of a user, including for example periods of rest (e.g., sleep) and periods of activity, using one or more signals from an analyte monitoring device.

In some variations, a method for detecting activity of a user may include receiving activity data from a wearable sensor and determining an onset of an activity state. This determination may be based on a first comparison of the received activity data to a first activity threshold over a first evaluation interval, and a second comparison of the received activity data to a second, different activity threshold applied over a second, different evaluation interval. The second evaluation interval may precede a portion of the first evaluation interval. The method may further include providing, via a display, activity information comprising a start time of the activity state based on the determined onset.

The activity data may comprise a step count detected over a plurality of discrete sampling intervals, such as intervals of about 5 minutes. In some variations, the method may include identifying a subset of the received activity data that meets a minimum activity threshold, wherein the subset is used for the determining step. The second evaluation interval may be shorter than the first evaluation interval; for example, the first interval may be about 10 minutes and the second may be about 5 minutes. Additionally, the first activity threshold may be greater than the second activity threshold.

In some variations, an end of the activity state may be determined based on a third, different activity threshold applied over a third evaluation interval. This may further involve evaluating the data over a fourth, different and subsequent evaluation interval. The activity information provided may consequently include an activity end time. Furthermore, the method may involve inserting an inactivity value correlated with gaps in the data exceeding a threshold duration, such as a gap of about 5 to 10 minutes.

The activity thresholds may be specifically configured to reduce false positive activity detections. In some variations, a remote device may receive the activity data. The method may also include aggregating the activity data received over discrete sampling intervals and classifying

the aggregated data as a low, moderate, or high activity state. Providing the information may include displaying a time-based visualization with segments aligned along a common timeline. The detected activity state may be a visually differentiated segment. In some variations, the timeline may simultaneously display sleep, medication, or glucose information. The system may also receive user input to hide categories of information or to select a temporal span. The displayed information may include a duration of the activity state, which may be classified by type and modified via user input.

Systems and non-transitory computer-readable media may also be provided to execute these processes. In some variations, a system may comprise a wearable sensor and a remote device configured to receive data, determine the onset of an activity state, and provide activity information via a display.

Additionally, methods for detecting inactivity of a user may include receiving activity data and light data from one or more wearable devices. A user-specific sleep reference time may be determined and used to transform a current time into a normalized time. An inactivity probability may then be generated based on the activity data, light data, and normalized time. An inactivity interval may be identified when this probability exceeds a threshold for a minimum evaluation interval. In some variations, the sleep reference time is based on user input regarding typical sleep or wake times. The normalized time may be constrained within a bounded range, such as −12 to +12 hours relative to the reference time.

A predictive model, such as a layered neural network, may receive the activity and light data to generate the inactivity probability. In some variations, the model may also consider proximity to the sleep reference time or data from intervals before and after the current time. The inactivity interval may be identified over a plurality of consecutive intervals, each of which may correspond to at least about one hour. The light data may be discretized into values, such as a scale of 1 to 5.

Furthermore, some variations relate to a method for determining inactivity by aggregating activity and light data within evaluation intervals preceding and following a target time to define a characterization interval. The light data may be quantized, and an inactivity probability may be determined associated with the target time. An inactivity interval, such as a sleep state, may be assigned if the probability exceeds a threshold. In some variations, an awake state is assigned if the probability does not persist for the minimum evaluation interval.

In some variations, a method for graphically representing physiological state information of a user may include receiving, at a processor, physiological data comprising activity intervals, sleep intervals, and analyte monitoring data for the user. The method may further include providing the activity intervals, the sleep intervals, and the analyte monitoring data via a graphical user interface (GUI). The GUI may comprise a timeline extending across a temporal span. The GUI may further comprise a first segment aligned in a first row along the timeline representing analyte monitoring data, such as glucose information.

In some variations, the temporal span may include a selectable temporal span such as a 12-hour view, a 24-hour view, or a multi-day view.

In some variations, the first segment may represent continuous analyte monitoring data over a first portion of the timeline. The first segment may comprise at least one of a first subsegment representing in-range analyte monitoring data or a second subsegment representing out-of-range analyte monitoring data. In some variations, the first segment may comprise both the first and second subsegments, and may be provided such that no temporal gaps exist between the first and second subsegments.

The GUI may also comprise a second segment aligned in a second row along the timeline representing a first activity interval and a third segment aligned in the second row representing a first sleep interval. The second and third segments may represent discrete, non-continuous traces of activity and sleep, respectively, that may be separated by a temporal gap over the first portion of the timeline. In some variations, the first activity interval and/or the first inactivity interval may be displayed with a start time and a duration. The method may involve receiving user input to modify at least one of a start time or a duration associated with the first activity interval or the first inactivity interval. Additionally, the first, second, and third segments may each comprise a unique color to facilitate visual differentiation.

In some variations, the GUI may be configured to re-render the second row with at least one of a modified second segment or a modified third segment without re-rendering the first row. The GUI may further include one or more user interface controls configured to selectively display or hide one or more categories of physiological information. These categories may comprise at least one of the analyte monitoring data, the activity intervals, the sleep intervals, or medication data. By providing these synchronized but visually distinct segments, the GUI may enhance a user's ability to correlate behavioral intervals with physiological responses.

Finally, a wearable analyte monitoring device may be provided. The device may comprise an ambient light sensor, a kinetic sensor to monitor step count, a clock, and a microcontroller. The microcontroller may include a trained machine learning model configured to determine a state of rest of the user based on signals from the light sensor, kinetic sensor, and clock. In some variations, the model may utilize time parameters inputted by the user. The device may further include a microneedle array for detecting an analyte and a communication controller to transmit state of rest data to a mobile application for display.

### BRIEF DESCRIPTION OF THE DRAWINGS

FIG. 1 depicts an illustrative schematic of an analyte monitoring system with a microneedle array.

FIG. 2A depicts an illustrative schematic of an analyte monitoring device.

FIG. 2B depicts an illustrative schematic of microneedle insertion depth in an analyte monitoring device.

FIG. 3A-FIG. 3D depict an upper perspective view, a side view, a bottom view, and an exploded view, respectively, of an analyte monitoring device.

FIG. 4A-FIG. 4E depict a perspective exploded view, a side exploded view, a lower perspective view, a side view, and an upper perspective view, respectively, of a sensor assembly in an analyte monitoring device.

FIG. 4F-FIG. 4H depict a perspective exploded view, a side exploded view, and a side view, respectively, of a sensor assembly in an analyte monitoring device.

FIG. 5A depicts an illustrative schematic of a microneedle array. FIG. 5B depicts an illustrative schematic of a microneedle in the microneedle array depicted in FIG. 5A.

FIG. 6 depicts an illustrative schematic of a microneedle array used for sensing multiple analytes.

FIG. 7A depicts a cross-sectional side view of a columnar microneedle having a tapered distal end. FIGS. 7B and 7C are images depicting perspective and detailed views, respectively, of an embodiment of the microneedle shown in FIG. 7A.

FIG. 8 depicts an illustrative schematic of a columnar microneedle having a tapered distal end.

FIG. 9A and FIG. 9B depict illustrative schematics of a microneedle array and a microneedle, respectively. FIG. 9C-FIG. 9F depict detailed partial views of an illustrative variation of a microneedle.

FIG. 10A and FIG. 10B depict an illustrative variation of a microneedle.

FIG. 11A and FIG. 11B depict illustrative schematics of a microneedle array configuration. FIG. 11C and FIG. 11D depict illustrative schematics of a microneedle array configuration.

FIG. 12A and FIG. 12B depict perspective and orthogonal views, respectively, of an illustrative variation of a die including a microneedle array.

FIG. 13 depicts a system block diagram of a sensor assembly in an analyte monitoring device.

FIG. 14 depicts a system block diagram of a rest detection model in an analyte monitoring device.

FIG. 15 depicts a block diagram of an activity detection process implementable in an analyte monitoring device.

FIG. 16A depicts a block diagram of an activity detection model in a remote device. FIG. 16B depicts a flow diagram of a method for detecting activity of a user via the activity detection model.

FIG. 17A depicts a block diagram of an inactivity detection model in a remote device. FIG. 17B depicts a flow diagram of a method for detecting inactivity of a user via the inactivity detection model. FIG. 17C depicts a flow diagram of another method for detecting inactivity of a user via the inactivity detection model.

FIG. 18 depicts a first example plot of glucose, physical activity, and sleep determined by an analyte monitoring device.

FIG. 19 depicts a second example plot of glucose, physical activity, and sleep determined by an analyte monitoring device.

FIG. 20 through FIG. 25 depict illustrative user interfaces that display analyte data.

FIG. 26A, FIG. 26B, FIG. 26C, and FIG. 26D depict illustrative user interfaces that display time-in-range analyte data.

FIG. 27A, FIG. 27B, FIG. 27C, FIG. 27D, and FIG. 27E depict illustrative user interfaces that display activity logging features.

FIG. 28A, FIG. 28B, FIG. 28C, and FIG. 28D depict illustrative user interfaces that display medication logging features.

FIG. 29A, FIG. 29B, FIG. 29C, FIG. 29D, FIG. 29E, FIG. 29F, FIG. 29G, FIG. 29H, and FIG. 29I depict illustrative user interfaces that display medication registration features.

FIG. 30A and FIG. 30B depict illustrative user interfaces that display sleep logging features.

FIG. 31 and FIG. 32 depict illustrative user interfaces that display analyte data with respect to a temporal scan.

FIG. 33 through FIG. 34D depict illustrative user interfaces that display features related to logging of events.

FIG. 35A, FIG. 35B, and FIG. 35C depict illustrative user interfaces that display side effects logging features.

FIG. 36A, FIG. 36B, FIG. 36C, and FIG. 36D depict illustrative user interfaces that display food logging features.

FIG. 37A through FIG. 41C depict illustrative user interfaces that display data with respect to timeline and goals.

FIG. 42A, FIG. 42B, FIG. 42C, FIG. 42D, FIG. 42E, FIG. 42F, FIG. 42G, FIG. 42H, FIG. 42I, and FIG. 42J depict illustrative user interfaces that provide data related to a wear session of an analyte monitoring device.

FIG. 43A, FIG. 43B, and FIG. 43C depict illustrative user interfaces that display goal editing features.

FIGS. 44, 45, and 46 depict a series of illustrative user registration user interfaces for creating a user account.

FIGS. 47-56 depict a series of illustrative onboarding user interfaces that guide a user through use and operation of an analyte monitoring device and mobile application.

FIGS. 57-61 depict a series of illustrative user interfaces that display time-in-range analyte data.

FIG. 62 depicts a series of illustrative user interfaces that provide access to informational resources.

FIG. 63 depict a series of illustrative user interfaces that display food logging features.

FIG. 64 depicts a first page of an exemplary glucose and lifestyle progress report.

FIG. 65 depicts a second page of ans exemplary glucose and lifestyle progress report.

### DETAILED DESCRIPTION

The term “a” or “an” refers to one or more of that entity, (e.g., can refer to plural referents). As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, an “inactivity state” refers to a state of intentional rest or sleep, and is distinct from periods during which a user is awake but not engaged in elevated physical activity. That is, an inactivity state is not merely the absence of an activity state, but rather a detected period of rest (e.g., characterized by low movement and low light) consistent with sleep. Periods during which a user is awake and engaged in normal daily activities (e.g., sitting, standing, walking at a non-elevated pace) but is not in an activity state or an inactivity state may be referred to herein as “baseline” periods or may simply not be assigned a physiological state classification.

Non-limiting examples of various aspects and variations of the invention are described herein and illustrated in the accompanying drawings.

Aspects of the current subject matter are directed to detecting physiological states of a user that may be represented as physiological state intervals (e.g., via a graphical user interface) for user interpretation. In some variations, physiological states like activity states and/or inactivity states of a user may be detected using signals from one or more monitoring devices. An activity state may comprise a period during which a user is active or exercising, while an inactivity state may comprise a period of rest, such as an intentional sedentary state. In some variations, the inactivity or rest state may be a sleep state. By identifying these states and representing them as corresponding activity intervals or inactivity intervals, the system may provide comprehensive physiological and/or behavioral information about a user. For example, data regarding these

intervals may be integrated with other health metrics like medication and analyte (e.g., glucose) levels, which may help users identify physiological and/or behavioral patterns and may improve health outcomes.

The detection of activity and inactivity states herein may be performed using data received from a variety of monitoring devices and device combinations. For example, a system may include a first wearable device (e.g., a wearable sensor) configured to provide activity data and a second wearable device configured to provide light data (e.g., ambient light levels). In some variations, the first and second wearable devices may be the same device (i.e., the same device may provide both the activity data and the light data), while in other variations, they may be separate and distinct devices worn by the user (i.e., one device may provide the activity data and a different device may provide the light data). In some variations, one of the devices may comprise an analyte monitoring device configured to detect at least one analyte (e.g., glucose) in a user. The analyte monitoring or other monitoring device may include a kinetic sensor (e.g., an accelerometer to monitor step count), an ambient light sensor, a temperature sensor, or a combination thereof.

In some variations, a system may include a remote device (e.g., a mobile phone, tablet, or cloud-based server) communicably coupled to the one or more monitoring devices. The remote device may be configured to receive activity data, light data, and/or analyte data from the one or more monitoring devices. The remote device may include a display for providing information regarding detected states, such as start times, durations, and time-based visualizations (e.g., via a graphical user interface). For instance, the remote device may receive activity data from a first device and light data from a second device (which, as noted above, may be the same or different devices), and may subsequently determine an inactivity state (e.g., a sleep state) by processing the data locally or via a remote processor.

In some variations, a graphical user interface (GUI) provided via the display may be configured to represent physiological state information in an interactive, time-aligned format. For example, the GUI may comprise a timeline extending across a selectable temporal span (e.g., 12 hours, 24 hours, or multiple days) that simultaneously displays activity intervals, sleep intervals, and analyte monitoring data. The GUI may be configured to receive user input to selectively display or hide specific categories of information, such as medication data or analyte level data, and may allow a user to adjust or modify detected intervals, such as a start time, end time, and/or duration of a sleep or activity interval. By presenting continuous analyte data alongside discrete, non-continuous traces of activity and sleep, the GUI may provide a visually differentiated and synchronized view of a user's health metrics, which may enhance the user's ability to correlate behavioral events with physiological responses.

The detection methods and systems described herein may provide several technical advantages over traditional monitoring systems. For example, the system may implement a multi-interval evaluation process that may enable the precise identification of an activity state “onset” or “start” and/or “end” even when activity data (e.g., step count data) is discrete, low-resolution, or otherwise imprecise. By evaluating activity levels across multiple distinct temporal evaluation intervals, the system may effectively filter out transient movements and may reduce false-positive detections. This may ensure that users are not prompted to log or review activity intervals that never actually occurred, which may improve the efficiency and reliability of the system.

Furthermore, the systems and methods described herein may address the technical challenge of accurately characterizing rest and sleep when point-in-time data (e.g., a single instance of zero activity and zero light) is insufficient to distinguish between sleep and wakeful inactivity. Because a user may be sedentary but awake (e.g., sitting or in a supine position), the system may analyze data over an extended characterization interval that spans both before and after a specific target time. This contextual analysis of data may allow the system to smooth out sensor noise and discontinuities, which may provide a more accurate determination of a user's actual physiological state.

To further enhance detection accuracy, the system may implement a temporal normalization process centered around user-specific references. By shifting temporal data relative to a user's typical behavior patterns, the system may create a clearer distinction between states of rest and states of wakefulness. This technical approach may ensure that the detected sleep matches the user's subjective experience and behavior more closely than standard population-based averages, which may effectively prevent the overestimation of rest intervals.

By providing accurate detection of activity and inactivity states, the systems and methods described herein may help users better understand their behavior patterns and make more informed lifestyle choices. For example, by correlating detected sleep periods with, for example, glucose readings, a user with diabetes may identify how their rest patterns affect their glycemic control and may adjust their sleep habits accordingly. Similarly, by correlating detected activity periods with glucose responses, the user may understand how different types or durations of exercise influence their blood sugar levels. This information may empower users to make behavioral changes—like adjusting meal timing relative to exercise, improving sleep habits, or increasing exercise—that may lead to better disease management and improved health outcomes. The activity and inactivity detection described herein may thus serve as a foundation for personalized health insights that extend beyond raw sensor data to actionable guidance for the user.

Generally, the systems herein may comprise one or more monitoring devices, each configured to monitor one or more physiological or environmental conditions, such as analyte levels, activity (e.g., user movement), light (e.g., ambient light), and/or temperature. A monitoring device may comprise at least one sensor configured to detect a physiological or environmental condition. In some variations, a monitoring device may comprise a plurality of sensors, each configured to detect a different physiological or environmental condition. For example, systems for detecting activity and/or inactivity of a user may comprise at least one of an activity monitoring device (e.g., comprising an accelerometer, gyroscope, and/or an inertial measurement unit), a light monitoring device (e.g., comprising a light sensor), a temperature monitoring device (e.g., comprising a temperature sensor), or an analyte monitoring device (e.g., comprising an analyte sensor). In some variations, a monitoring device may be a wearable device configured to be worn on a body of a user. In some variations, one or more monitoring devices herein may be communicably coupled to transmit data therebetween.

Additionally, the systems here may generally comprise a remote device (or a plurality of remote devices) to receive and process and/or analyze data from the one or more monitoring devices. A “remote device” may refer to any computing device that is communicably coupled to a monitoring device and is configured to receive data therefrom and perform processing thereon. A remote device may comprise a mobile computing device (e.g., a smartphone, tablet, or smartwatch), a cloud-based server, or a combination thereof. In some variations, processing may be distributed between a mobile computing device and a cloud-based server, and both may be considered remote devices.

In some variations, a remote device (e.g., a smartphone, smartwatch, or tablet) may comprise at least one processor and at least one memory storing instructions that, when executed by the processor, cause the remote device to perform the activity and inactivity detection methods described herein. In some variations, the one or more processors may comprise one or more of a central processing unit (CPU), a graphics processing unit (GPU), a digital signal processor (DSP), a microcontroller, a finite state machine (FSM), a machine learning processor, an application specific integrated circuit (ASIC), or a field programmable gate array (FPGA). In some variations, the one or more processors may execute software, hardware, or combinations thereof. In some variations, the remote device may be configured to receive and proceed raw data from a monitoring device. Additionally, or alternatively the remote device may be configured to receive processed data for analysis from a monitoring device.

The memory may comprise one or more non-transitory computer-readable media configured to store instructions and/or data. In some variations, the memory may comprise one or more of RAM, ROM, EEPROM, flash memory, or other volatile or non-volatile memory. The memory may store, for example, received sensor data, processed or normalized data, model parameters, outputs generated by the model, and/or instructions for performing the operations described herein.

The remote device may further comprise a display and physical interface components (e.g., touch-sensitive screens, buttons, or haptic engines) to enable the generation and operation of a mobile application/graphical user interface (GUI). The methods and models described herein may be implemented as a non-transitory computer-readable medium storing executable instructions. In some variations, such a computer-readable medium may be located on a monitoring device (e.g., an analyte monitoring device), the remote device, such as, for example, a mobile computing device and/or a cloud-based server, or a combination thereof.

Further, a remote device may comprise a communication interface configured to receive data from, and/or transmit data to, one or more monitoring devices. In some variations, the communication interface may support wired or wireless communication. For example, the communication interface may be configured to receive activity data and/or light data from one or more monitoring devices and to transmit activity or inactivity information to a remote server or other remote device.

Before providing additional details regarding aspects of detecting inactivity states and activity states, the following provides a description of some aspects of an analyte monitoring device that may be used for such detections. The following descriptions are meant to be exemplary, and aspects related to the described wearable analyte monitoring device consistent with the current subject matter are not limited to the examples described herein. Additionally, while an analyte monitoring device may be used as a source for activity or light data, the methods for activity and inactivity detection described herein need not be limited to systems requiring an analyte monitoring device and may be practiced with any combination of wearable and remote devices.

As generally described herein, an analyte monitoring system may include an analyte monitoring device that is worn by a user and includes one or more sensors for monitoring at least one analyte of a user. The sensors may, for example, include one or more electrodes configured to perform electrochemical detection of at least one analyte. The analyte monitoring device may communicate sensor data to a remote device (e.g., an external computing device or mobile computing device) for storage, display, and/or analysis of sensor data.

The wearable analyte monitoring device may be applied in any suitable location, though in some variations it may be desirable to avoid anatomical areas of thick or calloused skin (e.g., palmar and plantar regions), or areas undergoing significant flexion (e.g., olecranon or patella). Suitable wear sites may include, for example, on the arm (e.g., upper arm, lower arm), shoulder (e.g., over the deltoid), back of hands, neck, face, scalp, torso (e.g., on the back such as in the thoracic region, lumbar region, sacral region, etc. or on the chest or abdomen), buttocks, legs (e.g., upper legs, lower legs, etc.), and/or top of feet.

As described above, the working electrode is the electrode at which the oxidation and/or reduction reaction of interest occurs. In some variations, sensing may be performed at the interface of the working electrode and interstitial fluid located within the body (e.g., on an outer surface of the overall microneedle). In some variations, a working electrode may include an electrode material and a biorecognition layer in which a biorecognition element (e.g., enzyme) is immobilized on the working electrode to facilitate selective analyte quantification. In some variations, the biorecognition layer may also function as an interference-blocking layer and may help prevent endogenous and/or exogenous species from directly oxidizing (or reducing) at the electrode. In some variations, the biorecognition layer and the interference-blocking layer may be separate and distinct layers. In some variations, in addition to the biorecognition layer and/or the combined biorecognition and interference-blocking layer, an electrode protecting layer may be provided for additional protection of the electrode.

In some variations, the working electrode may include an electrode material and a biorecognition layer arranged at least partially over the electrode material, where the biorecognition layer includes an aptamer that selectively and reversibly binds an analyte. The biorecognition layer may include a conductive polymer layer and the aptamer, and the electrode material may include platinum. In some variations, the aptamer may be tethered to the conductive polymer layer via an amide linker. The amide linker may be formed through a reduction of a carboxyl group in the conductive polymer layer and an amine group covalently bound to a 3′ end or a 5′ end of the aptamer, or conversely of an amine group in the conductive polymer layer and a carboxyl group covalently bound to a 3′ end or a 5′ end of the aptamer. In some variations, the electrode material may include gold, and the aptamer may be tethered to the electrode material via a thiol link between the gold and a thiol group covalently bound to a 3′ end or a 5′ end of the aptamer. In some variations, the biorecognition layer may further include 6-mercapto-1-hexanol tethered to the gold via a thiol link. In some variations, the aptamer may be covalently bound to a redox-active molecule at the 3′ end or the 5′ end of the aptamer, such that selective binding of the cortisol to the aptamer and a resulting conformational change of the aptamer brings the redox-active molecule closer to or farther from a surface of the electrode material to facilitate or attenuate electron transfer between the redox-active molecule and the electrode material, thereby generating the sensor signal. In some variations, the redox-active molecule may be methylene blue or an anthraquinone.

Once the analyte monitoring device is inserted and warm-up and any calibration has completed, the analyte monitoring device may be ready for providing sensor measurements of a target analyte. The target analyte (and any requisite co-factor(s)) diffuses from the biological milieu, through the biocompatible and diffusion-limiting layers on the working electrode, and to the biorecognition layer including the biorecognition element. In the presence of a co-factor (if present), the biorecognition element may convert the target analyte to an electroactive product.

A bias potential may be applied between the working and reference electrodes of the analyte monitoring device, and an electrical current may flow from the counter electrode to maintain the fixed potential relationship between the working and reference electrodes. This causes the oxidation or reduction of the electroactive product, causing a current to flow between the working electrodes and counter electrodes. The current value is proportional to the rate of the redox reaction at the working electrode and, specifically, to the concentration of the analyte of interest according to the Cottrell relation.

The electrical current may be converted to a voltage signal by a transimpedance amplifier and quantized to a digital bitstream by means of an analog-to-digital converter (ADC). Alternatively, the electrical current may be directly quantized to a digital bitstream by means of a current-mode ADC. The digital representation of the electrical current may be processed in the embedded microcontroller(s) in the analyte monitoring device and relayed to the wireless communication module for broadcast or transmission (e.g., to one or more remote devices). In some variations, the microcontroller may perform additional algorithmic treatment to the data to improve the signal fidelity, accuracy, and/or calibration, etc.

In some variations, the digital representation of the electrical current, or sensor signal, may be correlated to an analyte measurement (e.g., glucose measurement) by the analyte monitoring device. For example, the microcontroller may execute a programmed routine in firmware to interpret the digital signal and perform any relevant algorithms and/or other analysis. Keeping the analysis on-board the analyte monitoring device may, for example, enable the analyte monitoring device to broadcast analyte measurement(s) to multiple devices in parallel, while ensuring that each connected device has the same information. Thus, generally, the user's target analyte (e.g., glucose) values may be estimated and stored in the analyte monitoring device and communicated to one or more remote devices.

A remote device (e.g., mobile computing device 102 described below) may include one or more processors executing a mobile application to handle data (e.g., display data and analyze data) and provide notifications related to analyte data, rest data, and activity data. As described herein, the analyte data, rest data (also referred to herein as sleep data and/or inactivity data), and activity data may be determined by the analyte monitoring device, the remote device, one or more additional remote servers or devices, or a combination thereof. The one or more processors executing the mobile application generate or provide an interface to allow for user viewing of data, user input of data, user logging of additional data, and user manipulation of data and displays.

In some variations, wireless communication between the analyte monitoring device and the remote device is utilized for the transfer of data between the devices. In some variations, near field communication (NFC) technology is utilized. As described herein, a communication controller of the analyte monitoring device may include an NFC tag integrated circuit (IC) or NFC module, which enables wireless connection through an NFC reader of the remote device. Alignment and/or contact between the mobile computing device and the analyte monitoring device may initiate and/or cause transfer of data between the devices. In some variations, the mobile application may generate a user interface or display instructing the user to align the remote device with the analyte monitoring device and, in some variations, select or tap a button to initiate the transfer of data between the devices. In some variations, the alignment initiates the transfer of data between the devices. In some variations, each time the remote device is aligned with the analyte monitoring device, data is transferred. In some variations, data is updated and/or available at a predefined rate of time. In some variations, alternate or additional types of wireless communication may be implemented between the devices to affect the transfer of data to the mobile computing device, which then uses the data to provide the various user interfaces described herein. In some variations, the data provided on the various user interfaces is from a single point of time. In some variations, the data is over a period of time, such as a wear session of the analyte monitoring device. In some variations, the data is updated as the wear session progresses. In addition to or alternative to data transfer between the analyte monitoring device and the remote device, the remote device may obtain data, through one or more wireless or wired connections, from one or more remote servers or devices.

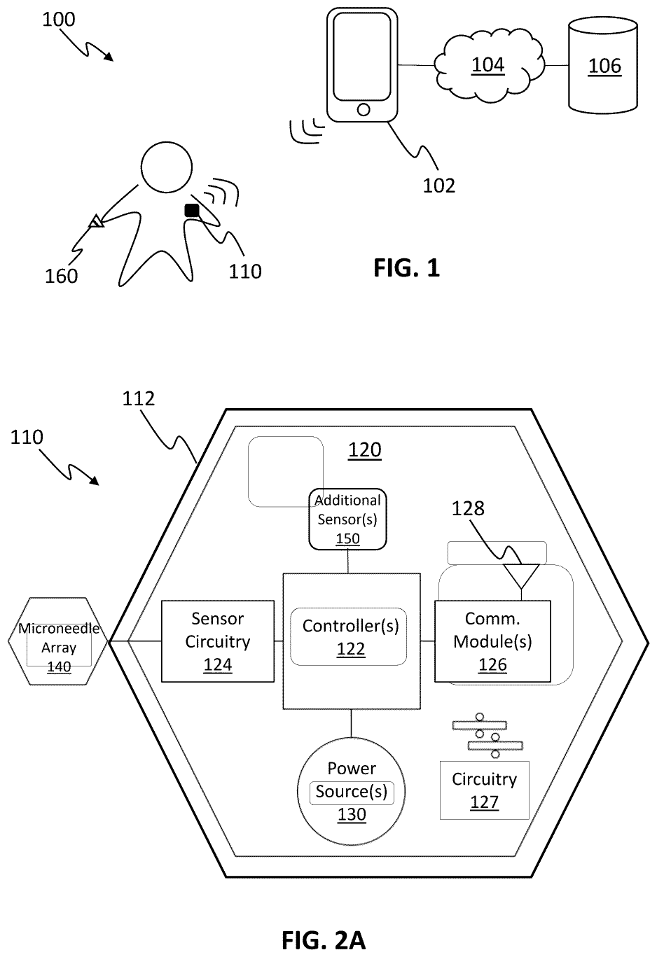

An exemplary analyte monitoring system is provided in FIG. 1. As shown, the analyte monitoring system 100 may include an analyte monitoring device 110 that is worn by a user, and the analyte monitoring device 110 may be a continuous analyte monitoring device (e.g., continuous glucose monitoring device). The analyte monitoring device 110 may include, for example, a microneedle array comprising at least one electrochemical sensor for detecting and/or measuring one or more analytes in body fluid of a user. The analyte monitoring device 110 may include one or more processors for performing analysis on sensor data, and/or a communication module (e.g., wireless communication module) configured to communicate sensor data to a mobile computing device 102 (e.g., smartphone) or other suitable computing device. In some variations, the mobile computing device 102 may include one or more processors executing a mobile application to handle sensor data (e.g., displaying data, analyzing data for trends, etc.) and/or provide suitable alerts or other notifications related to the sensor data and/or analysis thereof. It should be understood that while in some variations the mobile computing device 102 may perform sensor data analysis locally, other computing device(s) may alternatively or additionally remotely analyze sensor data and/or communicate information related to such analysis with the mobile computing device 102 (or other suitable user interface) for display to the user. Furthermore, in some variations the mobile computing device 102 may be configured to communicate sensor data and/or analysis of the sensor data over a network 104 to one or more storage devices 106 (e.g., server) for archiving data and/or other suitable information related to the user of the analyte monitoring device.

The analyte monitoring devices described herein have characteristics that improve a number of properties that are advantageous for a continuous analyte monitoring device such as a continuous glucose monitoring (CGM) device. For example, the analyte monitoring device described herein have improved sensitivity (amount of sensor signal produced per given concentration of target analyte), improved selectivity (rejection of endogenous and exogenous circulating compounds that can interfere with the detection of the target analyte), and improved stability to help minimize change in sensor response over time through storage and operation of the analyte monitoring device. Additionally, compared to conventional continuous analyte monitoring devices, the analyte monitoring devices described herein have a shorter warm-up time that enables the sensor(s) to quickly provide a stable sensor signal following implantation, as well as a short response time that enables the sensors(s) to quickly provide a stable sensor signal following a change in analyte concentration in the user. Furthermore, as described in further detail below, the analyte monitoring devices described herein may be applied to and function in a variety of wear sites, and provide for pain-free sensor insertion for the user. Other properties such as biocompatibility, sterilizability, and mechanical integrity are also optimized in the analyte monitoring devices described herein.

Although the analyte monitoring systems described herein may be described with reference to monitoring of glucose (e.g., in users with Type 2 diabetes, Type 1 diabetes), it should be understood that such systems may additionally or alternatively be configured to sense and monitor other suitable analytes. As described in further detail below, suitable target analytes for detection may, for example, include glucose, ketones, lactate, and cortisol. One target analyte may be monitored, or multiple target analytes may be simultaneously monitored (e.g., in the same analyte monitoring device). For example, monitoring of other target analytes may enable the monitoring of other indications such as stress (e.g., through detection of rising cortisol and glucose) and ketoacidosis (e.g., through detection of rising ketones).

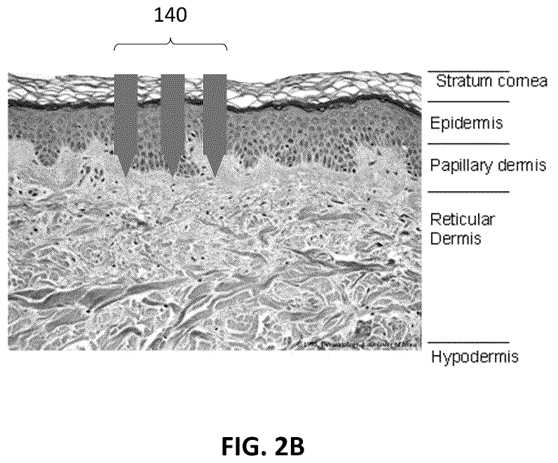

As shown in FIG. 2A, in some variations, an analyte monitoring device 110 may generally include a housing 112 and a microneedle array 140 extending outwardly from the housing. The housing 112, may, for example, be a wearable housing configured to be worn on the skin of a user such that the microneedle array 140 extends at least partially into the skin of the user. For example, the housing 112 may include an adhesive such that the analyte monitoring device 110 is a skin-adhered patch that is simple and straightforward for application to a user. The microneedle array 140 may be configured to puncture the skin of the user and include one or more electrochemical sensors (e.g., electrodes) configured for measuring one or more target analytes that are accessible after the microneedle array 140 punctures the skin of the user. In some variations, the analyte monitoring device 110 may be integrated or self-contained as a single unit, and the unit may be disposable (e.g., used for a period of time and replaced with another instance of the analyte monitoring device 110).

An electronics system 120 may be at least partially arranged in the housing 112 and include various electronic components, such as sensor circuitry 124 configured to perform signal processing (e.g., biasing and readout of electrochemical sensors, converting the analog signals from the electrochemical sensors to digital signals, etc.). The electronics system 120 may also include at least one microcontroller 122 for controlling the analyte monitoring device 110, at least one communication module 126, at least one power source 130, and/or other various suitable passive circuitry 127. The microcontroller 122 may, for example, be configured to interpret digital signals output from the sensor circuitry 124 (e.g., by executing a programmed routine in firmware), perform various suitable algorithms or mathematical transformations (e.g., calibration, etc.), and/or route processed data to and/or from the communication module 124. In some variations, the communication module 126 may include a suitable wireless transceiver (e.g., Bluetooth transceiver or the like) for communicating data with an external computing device 102 via one or more antennas 128. For example, the communication module 126 may be configured to provide unidirectional and/or bi-directional communication of data with an external computing device 102 that is paired with the analyte monitoring device 110. The power source 130 may provide power for the analyte monitoring device 110, such as for the electronics system. The power source 130 may include battery or other suitable source, and may, in some variations, be rechargeable and/or replaceable. Passive circuitry 127 may include various non-powered electrical circuitry (e.g., resistors, capacitors, inductors, etc.) providing interconnections between other electronic components, etc. The passive circuitry 127 may be configured to perform noise reduction, biasing and/or other purposes, for example. In some variations, the electronic components in the electronics system 120 may be arranged on one or more printed circuit boards (PCB), which may be rigid, semi-rigid, or flexible, for example. Additional details of the electronics system 120 are described further below.

In some variations, the analyte monitoring device 110 may further include one or more additional sensors 150 to provide additional information that may be relevant for user monitoring. For example, the analyte monitoring device 110 may further include at least one temperature sensor (e.g., thermistor) configured to measure skin temperature, thereby enabling temperature compensation for the sensor measurements obtained by the microneedle array electrochemical sensors.

The microneedle array 140 in the analyte monitoring device 110 is configured to puncture skin of a user. As shown in FIG. 2B, when the device 110 is worn by the user, the microneedle

array 140 may extend into the skin of the user such that electrodes on distal regions of the microneedles rest in the dermis. Specifically, in some variations, the microneedles may be designed to penetrate the skin and access the upper dermal region (e.g., papillary dermis and upper reticular dermis layers) of the skin, in order to enable the electrodes to access interstitial fluid that surrounds the cells in these layers. For example, in some variations, the microneedles may have a height generally ranging between at least 350 μm and about 515 μm. In some variations, one or more microneedles may extend from the housing such that a distal end of the electrode on the microneedle is located less than about 5 mm from a skin-interfacing surface of the housing, less than about 4 mm from the housing, less than about 3 mm from the housing, less than about 2 mm from the housing, or less than about 1 mm from the housing.

In contrast to traditional continuous analyte monitoring devices (e.g., CGM devices), which include sensors typically implanted between about 8 mm and about 10 mm beneath the skin surface in the subcutis or adipose layer of the skin, the analyte monitoring device 110 has a shallower microneedle insertion depth of about 0.25 mm (such that electrodes are implanted in the upper dermal region of the skin) that provides numerous benefits. These benefits include access to dermal interstitial fluid including one or more target analytes for detection, which is advantageous at least because at least some types of analyte measurements of dermal interstitial fluid have been found to closely correlate to those of blood. For example, it has been discovered that glucose measurements performed using electrochemical sensors accessing dermal interstitial fluid are advantageously highly linearly correlated with blood glucose measurements. Accordingly, glucose measurements based on dermal interstitial fluid are highly representative of blood glucose measurements.

Additionally, because of the shallower microneedle insertion depth of the analyte monitoring device 110, a reduced time delay in analyte detection is obtained compared to traditional continuous analyte monitoring devices. Such a shallower insertion depth positions the sensor surfaces in close proximity (e.g., within a few hundred micrometers or less) to the dense and well-perfused capillary bed of the reticular dermis, resulting in a negligible diffusional lag from the capillaries to the sensor surface. Diffusion time is related to diffusion distance according to t=x²/(2D) where is the diffusion time, x is the diffusion distance, and D is the mass diffusivity of the analyte of interest. Therefore, positioning an analyte sensing element twice as far away from the source of an analyte in a capillary will result in a quadrupling of the diffusional delay time. Accordingly, conventional analyte sensors, which reside in the very poorly vascularized adipose tissue beneath the dermis, result in a significantly greater diffusion distance from the vasculature in the dermis and thus a substantial diffusional latency (e.g., typically 5-20 minutes). In contrast, the shallower microneedle insertion depth of the analyte monitoring device 110 benefits from low diffusional latency from capillaries to the sensor, thereby reducing time delay in analyte detection and providing more accurate results in real-time or near real-time. For example, in some embodiments, diffusional latency may be less than 10 minutes, less than 5 minutes, or less than 3 minutes.

Furthermore, when the microneedle array rests in the upper dermal region, the lower dermis beneath the microneedle array includes very high levels of vascularization and perfusion to support the dermal metabolism, which enables thermoregulation (via vasoconstriction and/or vasodilation) and provides a barrier function to help stabilize the sensing environment around the microneedles. Yet another advantage of the shallower insertion depth is that the upper dermal layers lack pain receptors, thus resulting in a reduced pain sensation when the microneedle array punctures the skin of the user, and providing for a more comfortable, minimally-invasive user experience.

Thus, the analyte monitoring devices and methods described herein enable improved continuous monitoring of one or more target analytes of a user. For example, as described above, the analyte monitoring device may be simple and straightforward to apply, which improves ease-of-use and user compliance. Additionally, analyte measurements of dermal interstitial fluid may provide for highly accurate analyte detection. Furthermore, compared to traditional continuous analyte monitoring devices, insertion of the microneedle array and its sensors may be less invasive and involve less pain for the user. Additional advantages of other aspects of the analyte monitoring devices and methods are further described below.

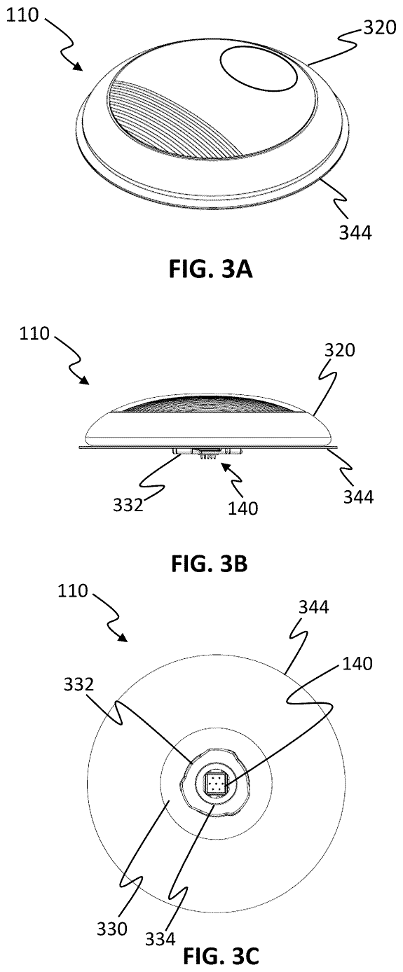

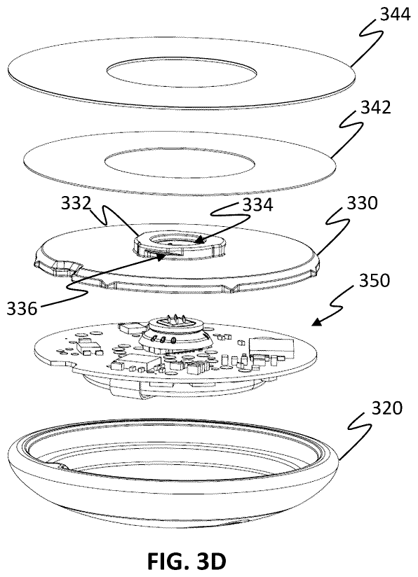

FIG. 3A-FIG. 3D depict aspects of the analyte monitoring device 110. FIG. 3A-FIG. 3D depict an upper perspective view, a side view, a bottom view, and an exploded view, respectively, of the analyte monitoring device 110.

The analyte monitoring device 110 may include a housing that at least partially surrounds or encloses other components (e.g., electronic components) of the analyte monitoring device 110, such as for protection of such components. For example, the housing may be configured to help prevent dust and moisture from entering the analyte monitoring device 110. In some variations, an adhesive layer may attach the housing to a surface (e.g., skin) of a user, while permitting the microneedle array 140 to extend outwardly from the housing and into the skin of the user. Furthermore, in some variations, the housing may generally include rounded edges or corners and/or be low-profile to reduce interference with clothing, etc. worn by the user.

For example, as shown in FIGS. 3A-3D, an example variation of the analyte monitoring device 110 may include a housing cover 320 and a base plate 330, configured to at least partially surround internal components of the analyte monitoring device 110. For example, the housing cover 320 and the base plate 330 may provide an enclosure for a sensor assembly 350 including the microneedle array 140 and electronic components. Once assembled, the microneedle array 140 extends outwardly from a portion of the base plate 330 in a skin-facing direction (e.g., an underside) of the analyte monitoring device 110.

The housing cover 320 and the base plate 330 may, for example, include one or more rigid or semi-rigid protective shell components that may couple together via suitable fasteners (e.g., mechanical fasteners), mechanically interlocking or mating features, and/or an engineering fit. The housing cover 320 and the base plate 330 may include radiused edges and corners and/or other atraumatic features. When coupled together, the housing cover 320 and the base plate 330 may form an internal volume that houses internal components, such as the sensor assembly 350. For example, the internal components arranged in the internal volume may be arranged in a compact, low-profile stack-up as the sensor assembly 350.

The analyte monitoring device 110 may include one or more adhesive layers to attach the analyte monitoring device 110 (e.g., the coupled together housing cover 320 and the base plate 330) to a surface (e.g., the skin) of a user. As shown in FIG. 3D, the one or more adhesive layers may include an inner adhesive layer 342 and an outer adhesive layer 344. The inner adhesive layer 342 may adhere to the base plate 330, and the outer adhesive layer 344 may adhere to the inner adhesive layer 342 and, on its outward facing side, provide an adhesive for adhering (e.g., temporarily) to the skin of the user. The inner adhesive layer 342 and the outer adhesive layer 344 together act as a double-sided adhesive for adhering the analyte monitoring device 110 to the skin of the user. The outer adhesive layer 344 may be protected by a release liner that the user removes to expose the adhesive prior to skin application. In some variations, a single adhesive layer is provided. In some variations, the outer adhesive layer 344 and/or the inner adhesive layer 342 may have a perimeter that extends farther than the perimeter or periphery of the housing cover 320 and the base plate 330. This may increase surface area for attachment and increase stability of retention or attachment to the skin of the user. The inner adhesive layer 342 and the outer adhesive layer 344 each have an opening that permits passage of the outwardly extending microneedle array 140, as further described below. The openings of the inner adhesive layer 342 and the outer adhesive layer 344 may generally align with one another but may, in some variations, differ in size such that one opening is smaller than the other opening. In some variations, the openings are substantially the same size.

The base plate 330 has a first surface (e.g., outwardly exposed surface) opposite a second surface and serves as a support and/or connection structure and as a protective cover for the sensor assembly 350. The base plate 330 is sized and shaped to attach to the housing cover 320. The base plate 330 may be shaped to securely fit within the housing cover 320 such that outer edges of the base plate 330 align with corresponding edges of an opening of the housing 320. The alignment may be such that there is no gap between the outer edges of the base plate 330 and the corresponding edges of the opening of the housing cover 320.

A connection member 332 may be formed in a central or near central region of the first surface of the base plate 330. The connection member 332 has a first surface substantially parallel to the first surface of the base plate 330. Sidewalls extend from edges of the first surface of the connection member 332 to the first surface of the base plate 330. A remaining portion of the first surface of the base plate 330 surrounding the connection member 332 may be flat or substantially flat. One or more connector features may extend outwardly from the sidewalls of the connection member 332 to releasably engage with corresponding connectors of a microneedle enclosure. The first surface and the sidewalls of the connection member 332 define, in part, a cavity. The cavity may be further defined through a portion of the base plate 330 adjacent (e.g., below) the connection member 332. The cavity has an opening, and is accessible, on the second surface of the base plate 330. An aperture 334 is formed through the first surface of the connection member 332. The aperture 334 may be sized and shaped such that the microneedle array 140 fits securely within and extends through the aperture 334. For example, sidewalls of the microneedle array 140 may align with corresponding sidewalls of the aperture 334. In some variations, the aperture 334 may be sized and shaped to correspond with an area surrounding the microneedle array 140. The openings in the inner adhesive layer 342 and the outer adhesive layer 344 are sized such that the connection member 332 extends through the openings without interfering with the inner adhesive layer 342 and the outer adhesive layer 344. For example, the diameter of the opening of the inner adhesive layer 342 and the diameter of the opening of the outer adhesive layer 344 is larger than that of the connection member 332.

Although the housing cover 320 and the base plate 330 depicted in FIGS. 3A-3D are substantially circular with the housing cover 320 having a dome shape, in other variations, the housing cover 320 and the base plate 330 may have any suitable shape. For example, in other variations the housing cover 320 and the base plate 330 may be generally prismatic and have an elliptical, triangular, rectangular, pentagonal, hexagonal, or other suitable shape. The outer adhesive layer 344 may extend outwardly from the housing cover 320 and the base plate 330 to extend beyond the perimeter of the housing cover 320. The outer adhesive layer 344 may be circular, as shown in FIGS. 3A-3D or may have an elliptical, triangular, rectangular, pentagonal, hexagonal, or other suitable shape and need not be the same shape as the housing cover 320 and/or the base plate 330.

In some variations, the analyte monitoring device 110 may provide user status, analyte monitoring device status, and/or other suitable information directly via a user interface (e.g., display, indicator lights, etc. as described below) on the analyte monitoring device 110. Thus, in contrast to analyte monitoring devices that may solely communicate information to a separate remote device (e.g., mobile phone, etc.) that in turn communicates the information to a user, in some variations such information may be directly provided by the analyte monitoring device 110.

Accordingly, in some variations, the housing cover 320 may include a user interface, such as an interface to provide information in a visual, audible, and/or tactile manner to provide information regarding user status and/or status of the analyte monitoring device, and/or other suitable information. Examples of user status that may be communicated via the user interface include information representative of analyte measurement in the user (e.g., below a predetermined target analyte measurement threshold or range, within a predetermined target analyte measurement range, above a predetermined target analyte measurement threshold or range, increase or decrease of analyte measurement over time, rate of change of analyte measurement, other information relating to trend of analyte measurements, other suitable alerts associated with analyte measurement, etc.). Examples of analyte monitoring device status that may be communicated via the user interface include device operation mode (e.g., associated with device warm-up state, analyte monitoring state, battery power status such as low battery, etc.), a device error state (e.g., operational error, pressure-induced sensing attenuation, fault, failure mode, etc.), device power status, device life status (e.g., anticipated sensor end-of-life), status of connectivity between device and a mobile computing device, and/or the like.

In some variations, the user interface may by default be in an enabled or “on” state to communicate such information at least whenever the analyte monitoring device 110 is performing analyte measurements or whenever the analyte monitoring device 110 is powered on, thereby helping to ensure that information is continuously available to the user. For example, user interface elements may communicate through a display or indicator light(s) (e.g., as described below) not only alerts to flag user attention or recommend remedial action, but also when user status and/or device status are normal. Accordingly, in some variations, a user is not required to perform an action to initiate a scan to learn their current analyte measurement level(s), and such information may always readily be available to the user. In some variations, however, a user may perform an action to disable the user interface temporarily (e.g., similar to a “snooze” button) such as for a predetermined amount of time (e.g., 30 minutes, 1 hour, 2 hours, etc.) after which the user interface is automatically reenabled, or until a second action is performed to reenable the user interface.

In some variations, the user interface of the housing cover 320 may include a display configured to visually communicate information. The display may, for example, include a display screen (e.g., LCD screen, OLED display, electrophoretic display, electrochromic display, etc.) configured to display alphanumeric text (e.g., numbers, letters, etc.), symbols, and/or suitable graphics to communicate information to the user. For example, the display screen may include a numerical information, textual information, and/or a graphics (e.g., sloped line, arrows, etc.) of information such as user status and/or status of the analyte monitoring device. For example, the display screen may include text or graphical representations of analyte measurement levels, trends, and/or recommendations (e.g., physical activity, reduced dietary intake, etc.).

Indicator light(s) on the display may be illuminated in one or more various manners to communicate different kinds of information. For example, an indicator light may be selectively illuminated on or off to communicate information (e.g., illumination “on” indicates one status, while illumination “off” indicates another status). An indicator light may be illuminated in a selected color or intensity to communicate information (e.g., illumination in a first color or intensity indicates a first status, while illumination in a second color or intensity indicates a second status). An indicator light may be illuminated in a selected temporal pattern to communicate information (e.g., illumination in a first temporal pattern indicates a first status, while illumination in a second temporal pattern indicates a second status). For example, an indicator light may be selectively illuminated in one of a plurality of predetermined temporal patterns that differ in illumination frequency (e.g., repeated illumination at a rapid or slow frequency), regularity (e.g., periodic repeated illumination vs. intermittent illumination), duration of illumination “on” time, duration of illumination “off” time, rate of change in illumination intensity, duty cycle (e.g., ratio of illumination “on” time to illumination “off” time), and/or the like, where each predetermined temporal pattern may indicate a respective status.

In some variations, a display may include multiple indicator lights that may be collectively illuminated in one or more predetermined illumination modes or sequences in accordance with one or more predetermined spatial and/or temporal patterns. For example, in some variations, some or all the indicator lights arranged on a display may be illuminated in synchrony or in sequence to indicate a particular status. Accordingly, the selected subset of indicator lights (e.g., the spatial arrangement of the indicator lights that are illuminated) and/or the manner in which they are illuminated (e.g., illumination order, illumination rate, etc.) may indicate a particular status. In some variations, a plurality of indicator lights may illuminate simultaneously or in sequence to increase the diversity of the color palette. For example, in some variations, red, green, and blue LEDs may be illuminated in rapid succession to create the impression of white light to a user.

In some variations, one or more of the above-described illumination modes may be combined in any suitable manner (e.g., combination of varying color, intensity, brightness, luminosity, contrast, timing, location, etc.) to communicate information.

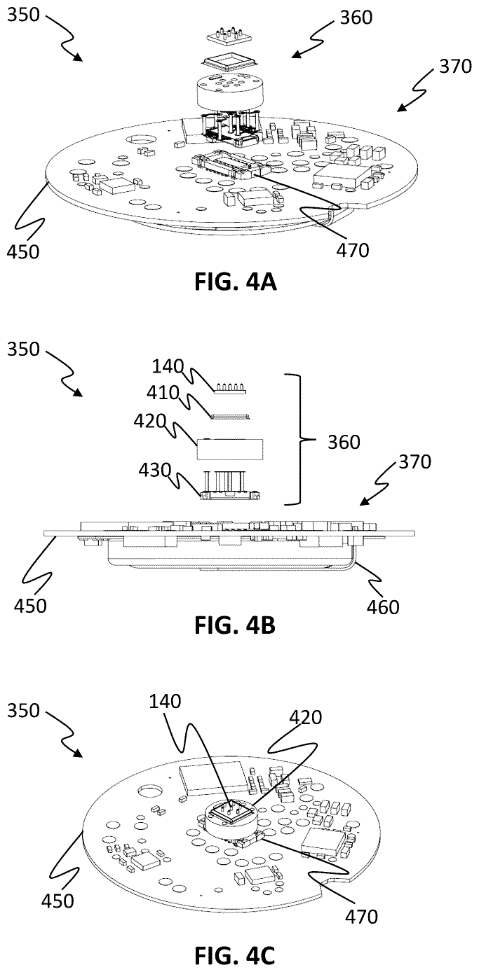

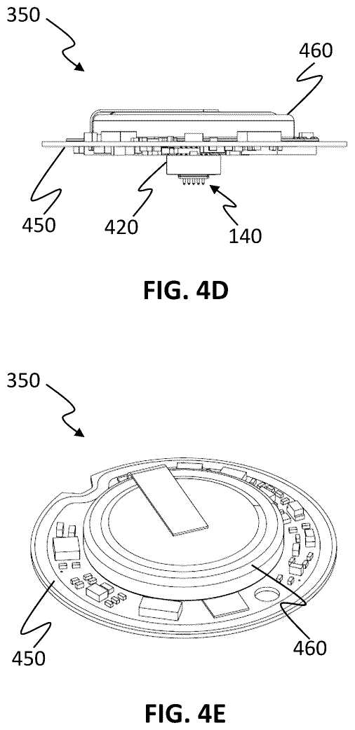

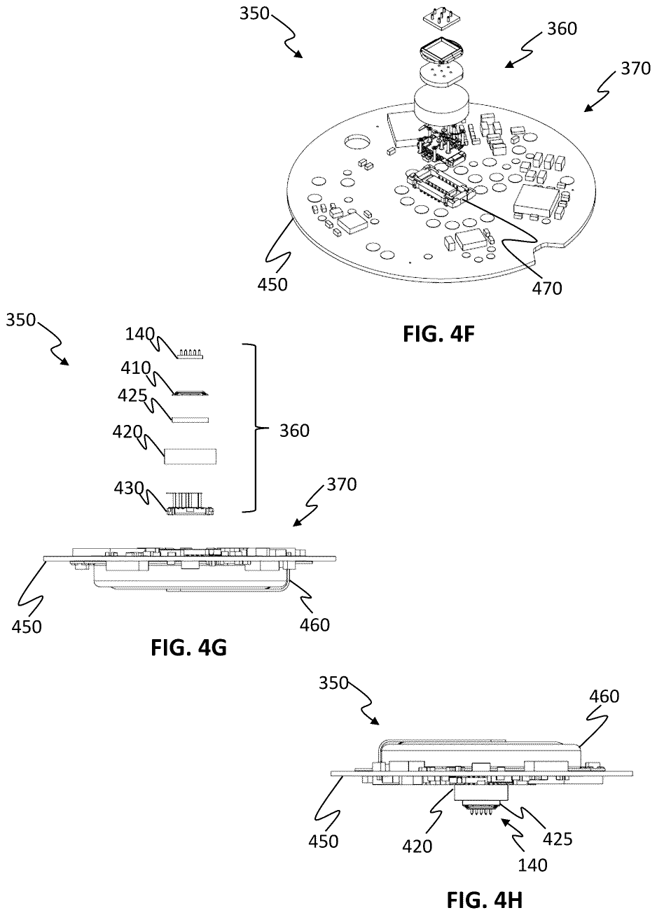

FIGS. 4A-4E depict aspects of the sensor assembly 350 of the analyte monitoring device 110 in a perspective exploded view, a side exploded view, a distal perspective view, a side view, and a proximal perspective view, respectively.

The sensor assembly 350 includes microneedle array components and electronic components to implement analyte detection and processing aspects of the microneedle array-based continuous analyte monitoring device 110 for the detection and measuring of an analyte. In some variations, the sensor assembly 350 is a compact, low-profile stack-up that is at least partially contained within the internal volume defined by the housing cover 320 and the base plate 330.

In some variations, the sensor assembly 350 includes a microneedle array assembly 360 and an electronics assembly 370 that connect to one another to implement the microneedle array analyte detection and processing aspects further described herein. In some variations, the electronics assembly 370 includes a first printed circuit board (PCB) 450 on which electronic components are connected, and the microneedle array assembly 360 includes a second printed circuit board (PCB) 420 on which the microneedle array 140 is connected.

In some variations, the microneedle array assembly 360 includes, in addition to the second PCB 420 and the microneedle array 140, an epoxy skirt 410 and a second PCB connector 430. The microneedle array 140 is coupled to a top side (e.g., outer facing or distal side) of the second PCB 420 so that the individual microneedles of the microneedle array 140 are exposed as described with reference to FIG. 3A-FIG. 3D. The second PCB connector 430 is coupled to a back or proximal side, opposite the top side, of the second PCB 420. The second PCB connector 430 may be an electromechanical connector and may communicatively couple to the first PCB 450 through a first PCB connector 470 on a top side (e.g., outer facing or distal side) of the first PCB 450 to allow for signal communication between the second PCB 420 and the first PCB 450. For example, signals from the microneedle array 140 may be communicated to the first PCB 450 through the second PCB 420, the second PCB connector 430, and the first PCB connector 470.

The second PCB 420 may in part determine the distance to which the microneedle array 140 protrudes from the back plate 330 of the housing. Accordingly, the height of the second PCB 420 may be selected to help ensure that the microneedle array 140 is inserted properly into a user's skin. During microneedle insertion, the first surface (e.g., outer facing surface) of the connection member 332 of the back plate 330 may act as a stop for microneedle insertion. If the second PCB 420 has a reduced height and its top surface is flush or nearly flush with the first surface of the connection member 332, then the connection member 332 may prevent the microneedle array 140 from being fully inserted into the skin.

In some variations, other components (e.g., electronic components such as sensors or other components) may also be connected to the second PCB 420. For example, the second PCB 420 may be sized and shaped to accommodate electronic components on the top side or the back side of the second PCB 420.

In some variations, the epoxy skirt 410 may be deposited along the edges (e.g., the outer perimeter) of the microneedle array 140 to provide a secure fit of the microneedle array 140 within the aperture 334 formed in the connection member 332 of the base plate 330 and/or to relieve the sharp edges along the microneedle array 140, as shown in FIG. 3B and FIG. 3C. For example, the epoxy skirt 410 may occupy portions of the aperture 334 not filled by the microneedle array 140 and/or portions of the cavity defined in the base plate 330 not filled by the second PCB 420. The epoxy skirt 410 may also provide a transition from the edges of the microneedle array 140 to the edge of the second PCB 420. In some variations, the epoxy skirt 410 may be replaced or supplemented by a gasket (e.g., a rubber gasket) or the like.

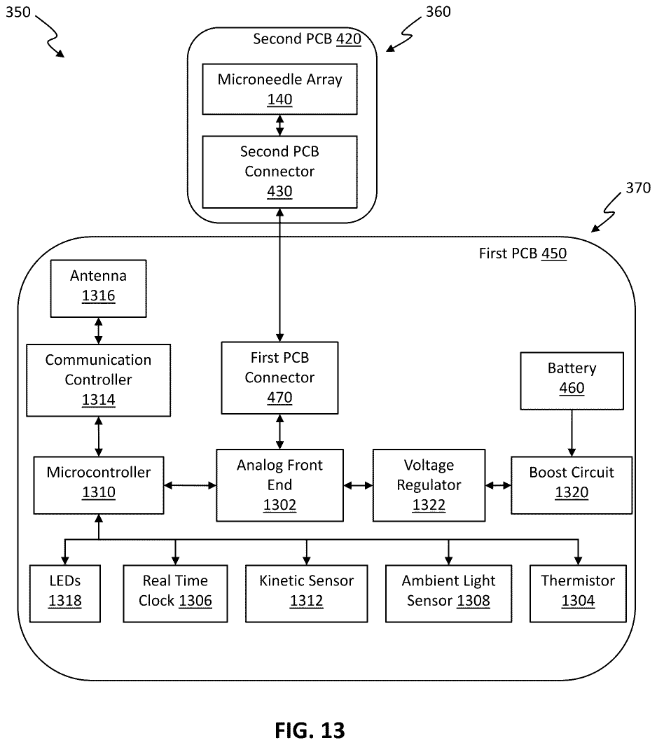

The electronics assembly 370, having the first PCB 450, includes a battery 460 coupled to a back side of the first PCB 450, opposite the top side on which the first PCB connector 470 is coupled. In some variations, the battery 460 may be coupled on the top side of the first PCB 450 and/or in other arrangements. Additional details of the electronics assembly are described with reference to FIG. 13.

FIGS. 4F-4H depict aspects of an alternate variation of the sensor assembly 350 of the analyte monitoring device 110. A perspective exploded view, a side exploded view, and a side view of the sensor assembly 350 are provided, respectively, in FIGS. 4F-4H.

As shown, in the sensor assembly 350, an additional PCB component, an intermediate PCB 425, is incorporated. In some variations, the intermediate PCB 425 is part of the microneedle array assembly 360 and is positioned between and connected to the second PCB 420 and the microneedle array 140. The intermediate PCB 425 may be added to increase the height of the microneedle array assembly 360 such that the microneedle array 140 extends at a further distance from the base plate 330, which may aid in insertion of the microneedle array 140 into the skin of a user. The microneedle array 140 is coupled to a top side (e.g., outer facing side) of the intermediate PCB 425 so that the individual microneedles of the microneedle array 140 are exposed as described with reference to FIG. 3A-FIG. 3D. The second PCB 420 is coupled to a back side, opposite the top side, of the intermediate PCB 425, and the second PCB connector 430 is coupled to a back side, opposite the top side, of the second PCB 420. The epoxy skirt 410 (which may be replaced or supplemented by a gasket of the like) provides a transition from the edges of the microneedle array 140 to the edge of the intermediate PCB 425.

The intermediate PCB 425 with the second PCB 420 in part determine the distance to which the microneedle array 140 protrudes through the aperture 334 of the back plate 330. The incorporation of the intermediate PCB 425 provides an additional height to help ensure that the microneedle array 140 is properly inserted into a user's skin. In some variations, the top side (e.g., outer facing side) of the intermediate PCB 425 extends through and out of the aperture 334 so that the first surface (e.g., top, exposed surface) of the connection member 332 surrounding the aperture 334 does not prevent the microneedle array from being fully inserted into the skin. In some variations, the top side (e.g., outer facing side) of the intermediate PCB 425 does not extend out of the aperture 334 but the increased height (by virtue of incorporating the intermediate PCB 425) ensures that the microneedle array 140 protrudes at a sufficient distance from the back plate 330 of the housing.

In some variations, a microneedle enclosure may be provided for releasable attachment to the analyte monitoring device 110. The microneedle enclosure may provide a protective environment or enclosure in which the microneedle array 140 may be safely contained, thereby ensuring the integrity of the microneedle array 140 during certain stages of manufacture and transport of the analyte monitoring device 110, prior to application of the analyte monitoring device 110. The microneedle enclosure is releasable or removable from the analyte monitoring device 110 to allow for the microneedle array 140 to be exposed and ready for insertion into the skin of the user, as further described herein.

In some variations, the microneedle enclosure, by providing an enclosed and sealed environment in which the microneedle array 140 may be contained, provides an environment in which the microneedle array 140 may be sterilized. For example, the microneedle enclosure with the microneedle array 140 may be subjected to a sterilization process, during which the sterilization penetrates the microneedle enclosure so that the microneedle array 140 is also sterilized. As the microneedle array 140 is contained in an enclosed environment, the microneedle array 140 remains sterilized until removed from the enclosed environment.

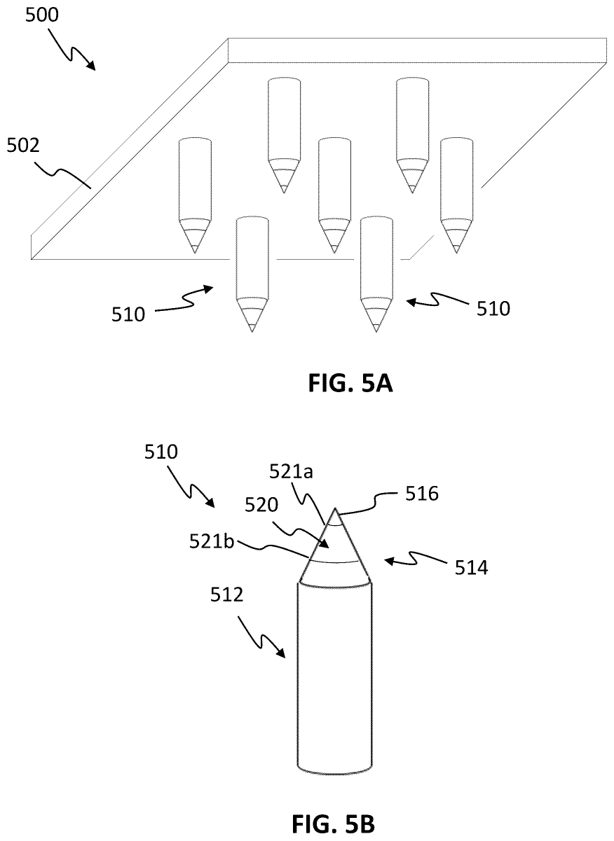

As shown in the schematic of FIG. 5A, in some variations, a microneedle array 510 for use in sensing an analyte may include one or more microneedles 510 projecting from a substrate surface 502. The substrate surface 502 may, for example, be a generally planar semiconductor (e.g. Silicon) substrate and one or more microneedles 510 may project orthogonally from the planar surface. Generally, as shown in FIG. 5B, a microneedle 510 may include a body portion 512 (e.g., shaft) and a tapered distal portion 514 configured to puncture skin of a user. In some variations, the tapered distal portion 514 may terminate in an insulated distal apex 516. The microneedle 510 may further include an electrode 520 on a surface of the tapered distal portion. In some variations, electrode-based measurements may be performed at the interface of the electrode and interstitial fluid located within the body (e.g., on an outer surface of the overall microneedle). In some variations, the microneedle 510 may have a solid core (e.g., solid body portion), though in some variations the microneedle 510 may include one or more lumens, which may be used for drug delivery or sampling of the dermal interstitial fluid, for example. Other microneedle variations, such as those described below, may similarly either include a solid core or one or more lumens.

The microneedle array 500 may be at least partially formed from a semiconductor (e.g., silicon) substrate and include various material layers applied and shaped using various suitable microelectromechanical systems (MEMS) manufacturing techniques (e.g., deposition and etching techniques), as further described below. The microneedle array may be reflow-soldered to a circuit board, similar to a typical integrated circuit. Furthermore, in some variations the microneedle array 500 may include a three electrode setup including a working (sensing) electrode having an electrochemical sensing coating (including a biorecognition element such as an aptamer or an enzyme) that enables detection of the analyte, a reference electrode, and a counter electrode. In other words, the microneedle array 500 may include at least one microneedle 510 that includes a working electrode, at least one microneedle 510 including a reference electrode, and at least one microneedle 510 including a counter electrode. Additional details of these types of electrodes are described in further detail below.

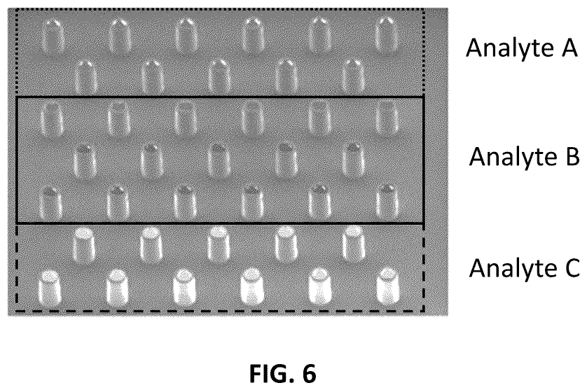

In some variations, the microneedle array 500 may include a plurality of microneedles that are insulated such that the electrode on each microneedle in the plurality of microneedles is individually addressable and electrically isolated from every other electrode on the microneedle array. The resulting individual addressability of the microneedle array 500 may enable greater control over each electrode's function, since each electrode may be separately probed. For example, the microneedle array 500 may be used to provide multiple independent measurements of a given analyte, which improves the device's sensing reliability and accuracy. Furthermore, in some variations the electrodes of multiple microneedles may be electrically connected to produce augmented signal levels. As another example, the same microneedle array 500 may additionally or alternatively be interrogated to simultaneously measure multiple analytes to provide a more comprehensive assessment of physiological status. For example, as shown in the schematic of FIG. 6, a microneedle array may include a portion of microneedles to detect s first analyte A, a second portion of microneedles to detect a second Analyte B, and a third portion of microneedles to detect a third Analyte C. It should be understood that the microneedle array may be configured to detect any suitable number of analytes (e.g., 1, 2, 3, 4, 5 or more, etc.), provided that at least one of the analytes is analyte.

In some variations of microneedles (e.g., microneedles with a working electrode), the electrode 520 may be located proximal to the insulated distal apex 516 of the microneedle. In other words, in some variations the electrode 520 does not cover the apex of the microneedle. Rather, the electrode 520 may be offset from the apex or tip of the microneedle. The electrode 520 being proximal to or offset from the insulated distal apex 516 of the microneedle advantageously provides more accurate sensor measurements. For example, this arrangement prevents concentration of the electric field at the microneedle apex 516 during manufacturing, thereby avoiding non-uniform electro-deposition of sensing chemistry on the electrode surface 520 that would result in faulty sensing. The electrode 520 may be configured to have an annular shape and may comprise a distal edge 521a and a proximal edge 521b.

As another example, placing the electrode 520 offset from the microneedle apex further improves sensing accuracy by reducing undesirable signal artefacts and/or erroneous sensor readings caused by stress upon microneedle insertion. The distal apex of the microneedle is the first region to penetrate into the skin, and thus experiences the most stress caused by the mechanical shear phenomena accompanying the tearing or cutting of the skin. If the electrode 520 were placed on the apex or tip of the microneedle, this mechanical stress may delaminate the electrochemical sensing coating on the electrode surface when the microneedle is inserted, and/or cause a small yet interfering amount of tissue to be transported onto the active sensing portion of the electrode. Thus, placing the electrode 520 sufficiently offset from the microneedle apex may improve sensing accuracy. For example, in some variations, a distal edge 521a of the electrode 520 may be located at least about 10 μm (e.g., between about 20 μm and about 30 μm) from the distal apex or tip of the microneedle, as measured along a longitudinal axis of the microneedle.

The body portion 512 of the microneedle 510 may further include an electrically conductive pathway extending between the electrode 520 and a backside electrode or other electrical contact (e.g., arranged on a backside of the substrate of the microneedle array). The backside electrode may be soldered to a circuit board, enabling electrical communication with the electrode 520 via the conductive pathway. For example, during use, the in-vivo sensing current (inside the dermis) measured at a working electrode is interrogated by the backside electrical contact, and the electrical connection between the backside electrical contact and the working electrode is facilitated by the conductive pathway. In some variations, this conductive pathway may be facilitated by a metal via running through the interior of the microneedle body portion (e.g., shaft) between the microneedle's proximal and distal ends. Alternatively, in some variations the conductive pathway may be provided by the entire body portion being formed of a conductive material (e.g., doped silicon). In some of these variations, the complete substrate on which the microneedle array 500 is built upon may be electrically conductive, and each microneedle 510 in the microneedle array 500 may be electrically isolated from adjacent microneedles 510 as described below. For example, in some variations, each microneedle 510 in the microneedle array 500 may be electrically isolated from adjacent microneedles 510 with an insulative barrier including electrically insulative material (e.g., dielectric material such as silicon dioxide) that surrounds the conductive pathway extending between the electrode 520 and backside electrical contact. For example, body portion 512 may include an insulative material that forms a sheath around the conductive pathway, thereby preventing electrical communication between the conductive pathway and the substrate. Other example variations of structures enabling electrical isolation among microneedles are described in further detail below.

Such electrical isolation among microneedles in the microneedle array permits the sensors to be individually addressable. This individually addressability advantageously enables independent and parallelized measurement among the sensors, as well as dynamic reconfiguration of sensor assignment (e.g., to different analytes). In some variations, the electrodes in the microneedle array can be configured to provide redundant analyte measurements, which is an advantage over conventional analyte monitoring devices. For example, redundancy can improve performance by improving accuracy (e.g., averaging multiple analyte measurement values from different microneedles which reduces the effect of extreme high or low sensor signals on the determination of analyte levels) and/or improving reliability of the device by reducing the likelihood of total failure.

In some variations, as described in further detail below with respective different variations of the microneedle, the microneedle array may be formed at least in part with suitable semiconductor and/or MEMS fabrication techniques and/or mechanical cutting or dicing. Such processes may, for example, be advantageous for enabling large-scale, cost-efficient manufacturing of microneedle arrays.

Described herein are further example variations of microneedle structures incorporating one or more of the above-described microneedle features for a microneedle array in an analyte monitoring device.

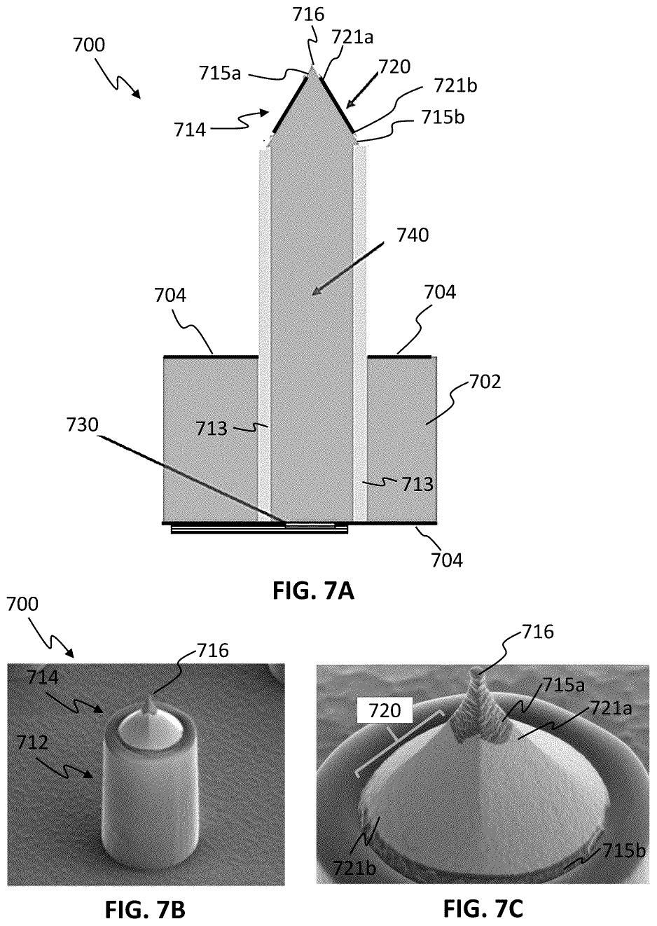

In some variations, a microneedle may have a generally columnar body portion and a tapered distal portion with an electrode. For example, FIGS. 7A-7C illustrate an example variation of a microneedle 700 extending from a substrate 702. FIG. 7A is a side cross-sectional view of a schematic of microneedle 700, while FIG. 7B is a perspective view of the microneedle 700 and FIG. 7C is a detailed perspective view of a distal portion of the microneedle 700. As shown in FIGS. 7B and 7C, the microneedle 700 may include a columnar body portion 712, a tapered distal portion 714 terminating in an insulated distal apex 716, and an annular electrode 720. The annular electrode 720 includes a conductive material (e.g., Pt, Ir, Au, Ti, Cr, Ni, combinations thereof, etc.) arranged on the tapered distal portion 714, such as, for example, on a segment thereof, and comprises a distal edge 721a and a proximal edge 721b. As shown in FIG. 7A, the annular electrode 720 may be proximal to (offset or spaced apart from) the distal apex 716. The annular electrode 720 may be electrically isolated from the distal apex 716 by a distal insulating surface 715a including an insulating material (e.g., SiO2). For example, the distal edge 721a of the annular electrode 720 may be proximate to a proximal edge of the distal insulating surface 715a of the insulated distal apex 716. In some variations, the distal edge 721a of the annular electrode 720 may be proximal to (e.g., just proximal to, adjacent, abutting) a proximal edge of the distal apex 716 (a proximal edge of the distal insulating surface 715a), while in other variations, the distal edge 721a of the annular electrode 720 may be distal to (e.g., just distal to, adjacent) the proximal edge of the insulated distal apex 716 (proximal edge of the distal insulating surface 715a), but may remain proximal to the apex itself. Accordingly, in some variations, the annular electrode 720 may overlie a portion of the distal insulating surface 715a, but may remain proximal to (and offset from) the insulated distal apex itself.

Also as shown in FIG. 7A, the proximal edge 721b of the annular electrode 720 may be distal to, and in some variations, offset or spaced apart from, the columnar body portion 712. In some variations, the proximal edge 721b of the annular electrode 720 may also be electrically isolated from the columnar body portion 712 by a second distal insulating surface 715b comprising an insulating material (e.g., SiO2) at a proximal end or region of the tapered distal portion 714. For example, the proximal edge 721b of the annular electrode 720 may be proximate to a distal edge of the second distal insulating surface 715b. In some variations, the proximal edge 721b of the annular electrode 720 may be proximal to (e.g., just proximal to, adjacent, abutting) a distal edge the second distal insulating surface 715b, while in other variations, the proximal edge 721b of the annular electrode 720 may be distal to (e.g., just distal to, adjacent) the distal edge of the second distal insulating surface 715b, but may remain proximal to the columnar body portion 712. Accordingly, in some variations, the annular electrode 720 may overlie a portion of the second distal insulating surface 715b but may remain proximal to (and offset from) the columnar body portion 712. As shown in FIG. 7A and in some other variations, the annular electrode 720 may be on only a segment of the surface of the tapered distal portion 714, and may or may not extend to the columnar boy portion 712.

The electrode 720 may be in electrical communication with a conductive core 740 (e.g., conductive pathway) passing along the body portion 712 to a backside electrical contact 730 (e.g., made of Ni/Au alloy) or other electrical pad in or on the substrate 702. For example, the body portion 712 may include a conductive core material (e.g., highly doped silicon). As shown in FIG. 7A, in some variations, an insulating moat 713 including an insulating material (e.g., SiO2) may be arranged around (e.g., around the perimeter) of the body portion 712 and extend at least partially through the substrate 702. Accordingly, the insulating moat 713 may, for example, help prevent electrical contact between the conductive core 740 and the surrounding substrate 702. The insulating moat 713 may further extend over the surface of the body portion 712. Upper and/or lower surfaces of the substrate 702 may also include a layer of substrate insulation 704 (e.g., SiO2). Accordingly, the insulation provided by the insulating moat 713 and/or substrate insulation 704 may contribute at least in part to the electrical isolation of the microneedle 700 that enables individual addressability of the microneedle 700 within a microneedle array. Furthermore, in some variations the insulating moat 713 extending over the surface of the body portion 712 may function to increase the mechanical strength of the microneedle 700 structure.

The microneedle 700 may be formed at least in part by suitable MEMS fabrication techniques such as plasma etching, also called dry etching. For example, in some variations, the insulating moat 713 around the body portion 712 of the microneedle may be made by first forming a trench in a silicon substrate by deep reactive ion etching (DRIE) from the backside of the substrate, then filling that trench with a sandwich structure of SiO2/polycrystalline silicon (poly-Si)/SiO2 by low pressure chemical vapor deposition (LPCVD) or other suitable process. In other words, the insulating moat 713 may passivate the surface of the body portion 712 of the microneedle, and continue as a buried feature in the substrate 702 near the proximal portion of the microneedle. By including largely compounds of silicon, the insulating moat 713 may provide good fill and adhesion to the adjoining silicon walls (e.g., of the conductive core 740, substrate 702, etc.). The sandwich structure of the insulating moat 713 may further help provide excellent matching of coefficient of thermal expansion (CTE) with the adjacent silicon, thereby advantageously reducing faults, cracks, and/or other thermally-induced weaknesses in the insulating structure 713.

The tapered distal portion may be fashioned out by an isotropic dry etch from the frontside of the substrate, and the body portion 712 of the microneedle 700 may be formed from DRIE. The frontside metal electrode 720 may be deposited and patterned on the distal portion by specialized lithography (e.g., electron-beam evaporation) that permits metal deposition in the desired annular region for the electrode 720 without coating the distal apex 716. Furthermore, the backside electrical contact 730 of Ni/Au may be deposited by suitable MEMS manufacturing techniques (e.g., sputtering).

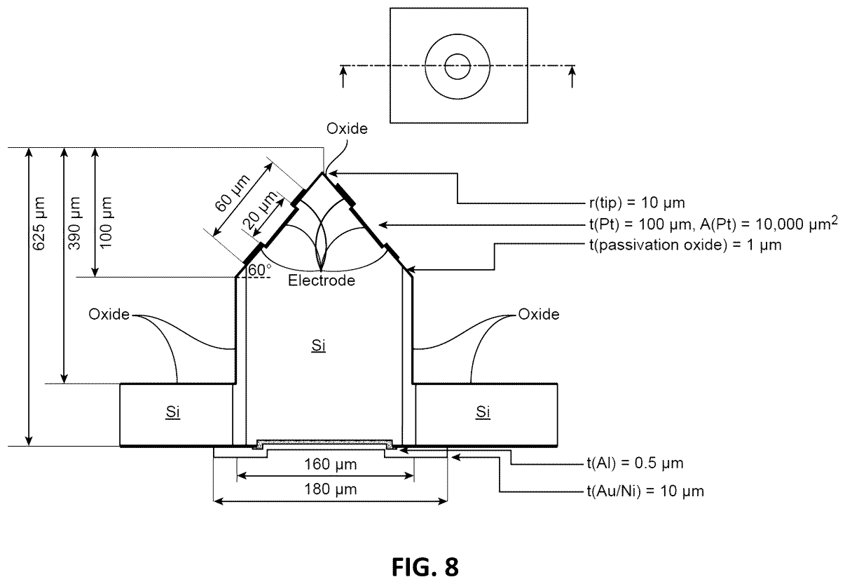

The microneedle 700 may have any suitable dimensions. By way of illustration, the microneedle 700 may, in some variations, have a height of between about 300 μm and about 500 μm. In some variations, the tapered distal portion 714 may have a tip angle between about 60 degrees and about 80 degrees, and an apex diameter of between about 1 μm and about 15 μm. In some variations, the surface area of the annular electrode 720 may include between about 9,000 μm² and about 11,000 μm², or about 10,000 μm². FIG. 8 illustrates various dimensions of an example variation of a columnar microneedle with a tapered distal portion and annular electrode, similar to microneedle 700 described above. As with the microneedle 700 described above, the columnar microneedle of FIG. 8 comprises a columnar body portion, a tapered distal portion terminating in an insulated distal apex, a contact trench formed within the tapered distal portion, and an annular electrode (denoted by “Pt” in FIG. 8) that is arranged on the tapered distal portion and overlays the contact trench. The annular electrode may comprise a conductive material (e.g., Pt, Ir, Au, Ti, Cr, Ni, combinations thereof, etc.). In some variations, the contact trench may have a width of about 1 μm, about 2 μm, about 3 μm, about 4 μm, about 5 μm, about 10 μm, about 15 μm, about 20 μm, about 25 μm, about 30 μm, about 35 μm, about 40 μm, about 45 μm, about 50 μm, or, as shown in FIG. 8, about 20 um. The annular electrode may comprise a distal edge and a proximal edge, and in some variations, a distance between the distal edge and the proximal edge of the annular electrode may be about 20 μm, about 30 μm, about 40 μm about 50 μm, about 60 μm, about 70 μm, about 80 μm, about 90 μm, about 100 μm, or, as shown in FIG. 8, about 60 82 m. In some variations, and as shown in FIG. 8 by the dimensional callouts 60 μm and 20 μm, the annular electrode may overlie the contact trench and, in some instances, a portion of the insulating surfaces (denoted by “Oxide” in FIG. 8) of the tapered distal portion.

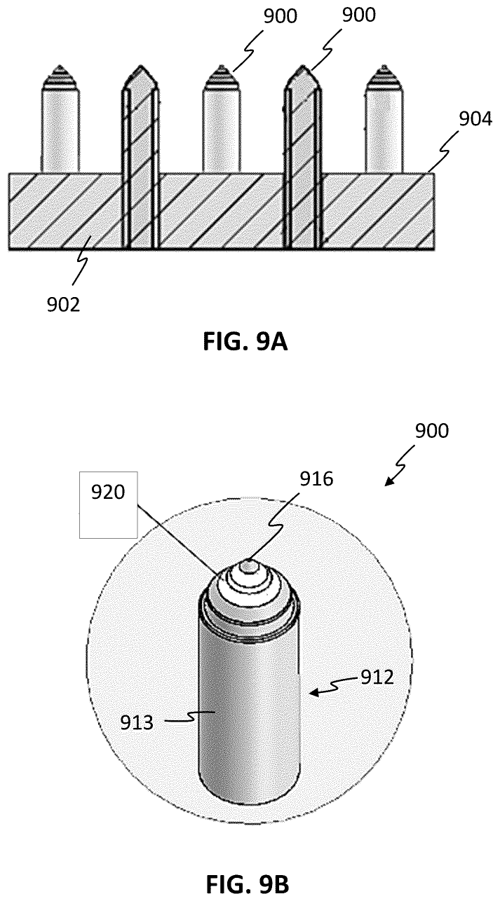

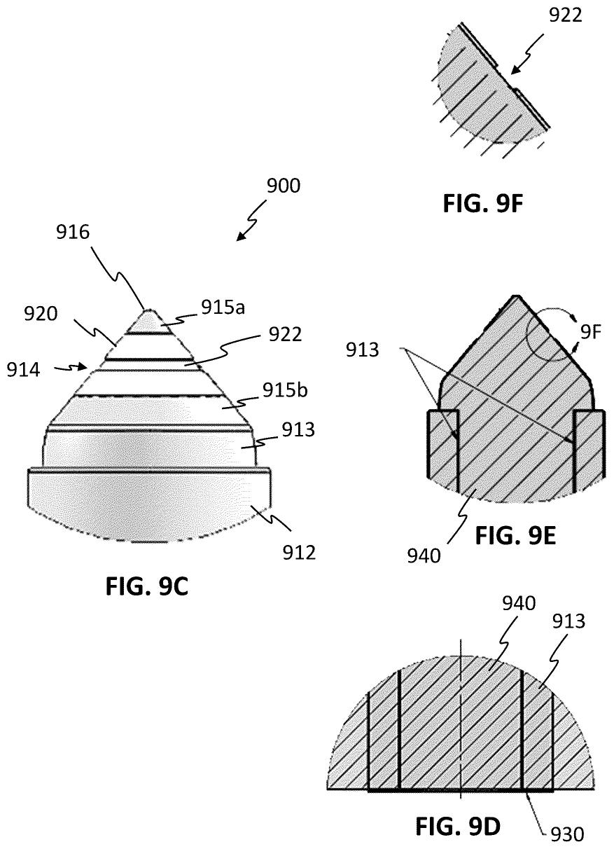

FIGS. 9A-9F illustrate another example variation of a microneedle 900 having a generally columnar body portion extending from a substrate 902 having a top surface 904. The microneedle 900 may be similar to microneedle 700 as described above, except as described below. For example, as shown in FIG. 9B, like the microneedle 700, the microneedle 900 may include a columnar body portion 912, and a tapered distal portion arranged on a cylinder 913 and terminating in an insulated distal apex 916. The cylinder 913 may be insulated and have a smaller diameter than the columnar body portion 912. The microneedle 900 may further include an annular electrode 920 that includes a conductive material and is arranged on the tapered distal portion at a location proximal to (or offset or spaced apart from) the distal apex 916. The electrode 920 may be in electrical communication with a conductive core 940 (e.g., conductive pathway) passing along the body portion 912 to a backside electrical contact 930 (e.g., made of Ni/Au alloy) or other electrical pad in or on the substrate 902. Other elements of microneedle 900 as shown in FIGS. 9A-9F have numbering similar to corresponding elements of microneedle 700.

As can most easily be seen in FIGS. 9B, 9C and 9F, the tapered distal portion 914, and more specifically, the electrode 920 on the tapered distal portion 914 of the microneedle 900, may include a tip contact trench 922. This contact trench may be configured to establish ohmic contact between the electrode 920 and the underlying conductive core 940 of the microneedle. In some variations, the shape of the tip contact trench 922 may include an annular recess formed in the surface of the tapered distal portion 914. In some variations, the shape of the tip contact trench 922 may include an annular recess formed in the surface of the conductive core 940 (e.g., into the body portion of the microneedle, or otherwise in contact with a conductive pathway in the body portion). In some variations, the tip contact trench 922 may be formed in the insulating material on the tapered distal portion 914, and may have a depth about equal to the thickness of the insulating material (e.g., the distal insulating surface 915a and/or the second distal insulating surface 915b). In some instances, the depth of the contact trench may be greater than the thickness of the insulating material such that the contact trench extends beyond a surface of the conductive core 940 (e.g., into the conductive core 940). The electrode 920 may overlie the tip contact trench 922 such that ohmic contact is established between the electrode 920 and the conductive core 940. In some variations, the electrode 920 may extend beyond the tip contact trench 922 such that when the electrode 920 material is deposited onto the conductive core 940, the electrode 920 with the tip contact trench 922 may have a stepped profile when viewed from the side. The tip contact trench 922 may thus advantageously help ensure contact between the electrode 920 and the underlying conductive core 940. Any of the other microneedle variations described herein may also have a similar tip contact trench to help ensure contact between the electrode (which may be, for example, a working electrode, reference electrode, counter electrode, etc.) with a conductive pathway within the microneedle.

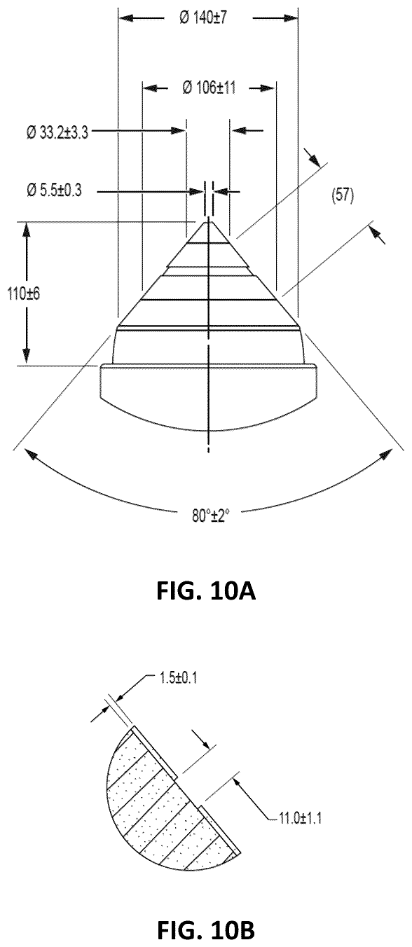

FIGS. 10A and 10B illustrate additional various dimensions of an example variation of a columnar microneedle with a tapered distal portion and annular electrode, similar to microneedle 900 described above. For example, the variation of the microneedle shown in FIGS. 10A and 10B may have a tapered distal portion generally having a taper angle of about 80 degrees (or between about 78 degrees and about 82 degrees, or between about 75 degrees and about 85 degrees), and a cone diameter of about 140 μm (or between about 133 μm and about 147 μm, or between about 130 μm and about 150 μm). The cone of the tapered distal portion may be arranged on a cylinder such that the overall combined height of the cone and cylinder is about 110 μm (or between about 99 μm and about 116 μm, or between about 95 μm and about 120 μm). The annular electrode on the tapered distal portion may have an outer or base diameter of about 106 μm (or between about 95 μm and about 117 μm, or between about 90 μm and about 120 μm), and an inner diameter of about 33.2 μm (or between about 30 μm and about 36 μm, or between about 25 μm and about 40 μm). The length of the annular electrode, as measured along the slope of the tapered distal portion, may be about 57 μm (or between about 55 μm and about 65 μm), and the overall surface area of the electrode may be about 12,700 μm² (or between about 12,500 μm² and about 12,900 μm², or between about 12,000 μm² and about 13,000 μm²). As shown in FIG. 10B, the electrode may furthermore have a tip contact trench extending around a central region of the cone of the tapered distal portion, where the contact may have a width of about 11 μm (or between about 5 μm and about 50 μm, between about 10 μm and about 12 μm, or between about 8 μm and about 14 μm) as measured along the slope of the tapered distal portion, and a trench depth of about 1.5 μm (or between about 0.1 μm and about 5 μm, or between about 0.5 μm and about 1.5 μm, or between about 1.4 μm and about 1.6 μm, or between about 1 μm and about 2 μm). The microneedle has an insulated distal apex having a diameter of about 5.5 μm (or between about 5.3 μm and about 5.8 μm, or between about 5 μm and about 6 μm).

As described above, each microneedle in the microneedle array may include an electrode. In some variations, multiple distinct types of electrodes may be included among the microneedles in the microneedle array. For example, in some variations the microneedle array may function as an electrochemical cell operable in an electrolytic manner with three types of electrodes. In other words, the microneedle array may include at least one working electrode, at least one counter electrode, and at least one reference electrode. Thus, the microneedle array may include three distinct electrode types, though one or more of each electrode type may form a complete system (e.g., the system might include multiple distinct working electrodes). Furthermore, multiple distinct microneedles may be electrically joined to form an effective electrode type (e.g., a single working electrode may be formed from two or more connected microneedles with working electrode sites). Each of these electrode types may include a metallization layer and may include one or more coatings or layers over the metallization layer that help facilitate the function of that electrode.

Generally, the working electrode is the electrode at which oxidation and/or reduction reaction of interest occurs for detection of an analyte of interest. The counter electrode functions to source (provide) or sink (accumulate) the electrons, via an electrical current, that are required to sustain the electrochemical reaction at the working electrode. The reference electrode functions to provide a reference potential for the system; that is, the electrical potential at which the working electrode is biased is referenced to the reference electrode. A fixed, time-varying, or at least controlled potential relationship is established between the working and reference electrodes, and within practical limits no current is sourced from or sinked to the reference electrode. Additionally, to implement such a three-electrode system, the analyte monitoring device may include a suitable potentiostat or electrochemical analog front end to maintain a fixed potential relationship between the working electrode and reference electrode contingents within the electrochemical system (via an electronic feedback mechanism), while permitting the counter electrode to dynamically swing to potentials required to sustain the redox reaction of interest.

Multiple microneedles (e.g., any of the microneedle variations described herein, each of which may have a working electrode, counter electrode, or reference electrode as described above) may be arranged in a microneedle array. Considerations of how to configure the microneedles include factors such as desired insertion force for penetrating skin with the microneedle array, optimization of electrode signal levels and other performance aspects, manufacturing costs and complexity, etc.

For example, the microneedle array may include multiple microneedles that are spaced apart at a predefined pitch (distance between the center of one microneedle to the center of its nearest neighboring microneedle). In some variations, the microneedles may be spaced apart with a sufficient pitch so as to distribute force (e.g., avoid a “bed of nails” effect) that is applied to the skin of the user to cause the microneedle array to penetrate the skin. As pitch increases, force required to insert the microneedle array tends to decrease and depth of penetration tends to increase. However, it has been found that pitch only begins to affect insertion force at low values (e.g., less than about 150 μm). Accordingly, in some variations the microneedles in a microneedle array may have a pitch of at least 200 μm, at least 300 μm, at least 400 μm, at least 500 μm, at least 600 μm, at least 700 μm, or at least 750 μm. For example, the pitch may be between about 200 μm and about 800 μm, between about 300 μm and about 700 μm, or between about 400 μm and about 600 μm. In some variations, the microneedles may be arranged in a periodic grid, and the pitch may be uniform in all directions and across all regions of the microneedle array. Alternatively, the pitch may be different as measured along different axes (e.g., X, Y directions) and/or some regions of the microneedle array may include a smaller pitch while other may include a larger pitch.

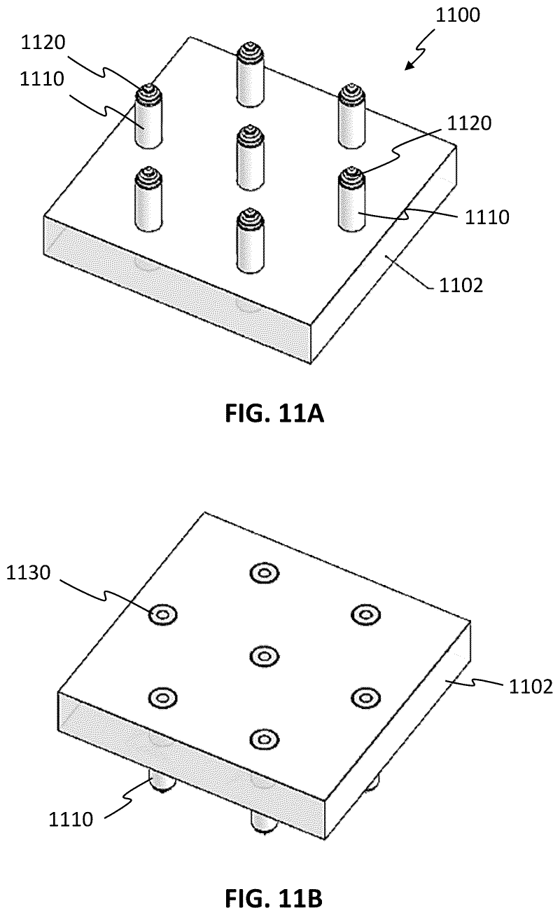

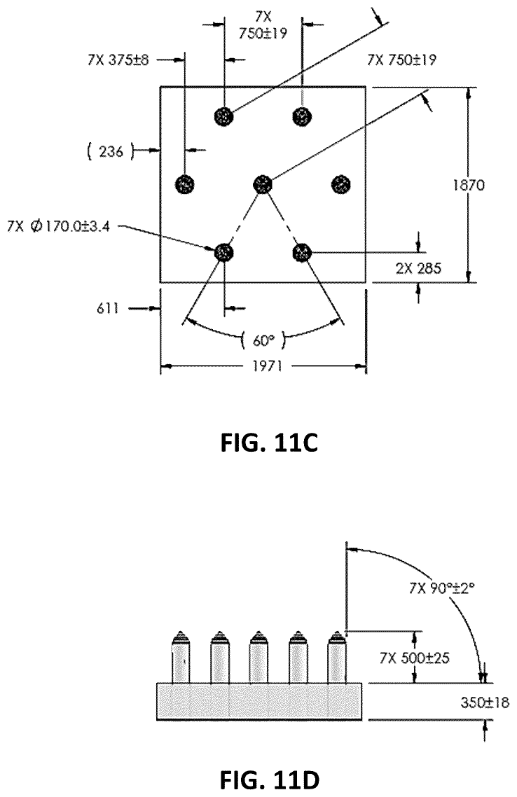

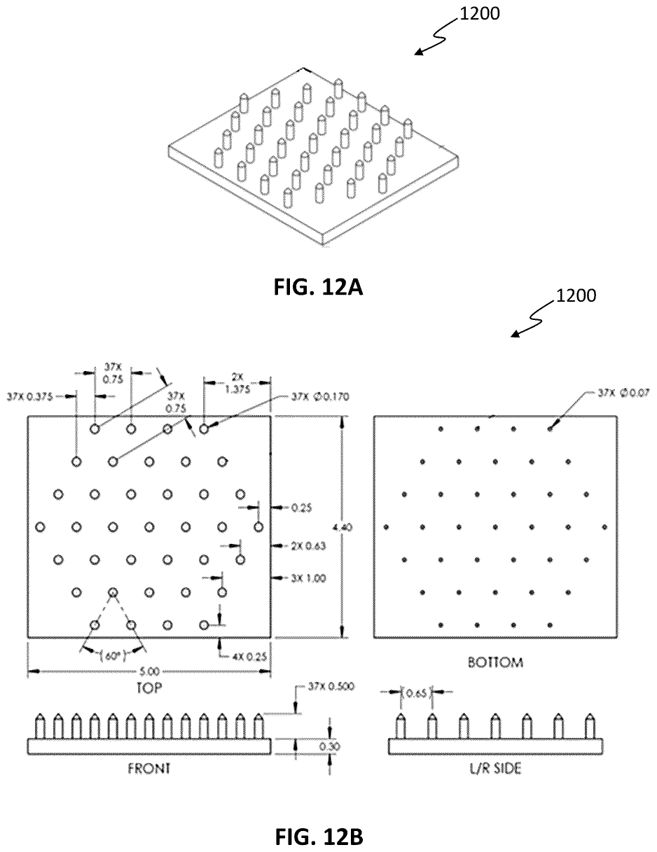

Furthermore, for more consistent penetration and in some variations, microneedles may be spaced equidistant from one another (e.g., same pitch in all directions). To that end, in some variations, the microneedles in a microneedle array may be arranged in a hexagonal configuration as shown in FIGS. 11A-11C and 12A-12B. Alternatively, the microneedles in a microneedle array may arranged in a rectangular array (e.g., square array), or in another suitable symmetrical manner.

FIGS. 11A and 11B depict perspective views of an illustrative schematic of seven microneedles 1110 arranged in an example variation of a microneedle array 1100. The seven microneedles 1110 are arranged in a hexagonal array on a substrate 1102. As shown in FIG. 11A, the electrodes 1120 are arranged on distal portions of the microneedles 1110 extending from a first surface of the substrate 1102. As shown in FIG. 11B, proximal portions of the microneedles 1110 are conductively connected to respective backside electrical contacts 1130 on a second surface of the substrate 1102 opposite the first surface of the substrate 1102. FIGS. 11C and 11D depict plan and side views of an illustrative schematic of a microneedle array similar to microneedle array 1100. As shown in FIGS. 11C and 11D, the seven microneedles are arranged in a hexagonal array with an inter-needle center-to-center pitch of about 750 μm between the center of each microneedle and the center of its immediate neighbor in any direction. In other variations the inter-needle center-to-center pitch may be, for example, between about 700 μm and about 800 μm, or between about 725 μm and about 775 μm. The microneedles may have an approximate outer shaft diameter of about 170 μm (or between about 150 μm and about 190 μm, or between about 125 μm and about 200 μm) and a height of about 500 μm (or between about 475 μm and about 525 μm, or between about 450 μm and about 550 μm).

FIGS. 12A and 12B depict an illustrative schematic of 37 microneedles arranged in an example variation of a microneedle array 1200. The 37 microneedles may, for example, be arranged in a hexagonal array with an inter-needle center-to-center pitch of about 750 μm (or between about 700 μm and about 800 μm, or between about 725 μm and about 775 μm) between the center of each microneedle and the center of its immediate neighbor in any direction. FIG. 12A depicts an illustrative schematic of an example variation of a die including the microneedle arrangement. Example dimensions of the die (e.g., about 4.4 mm by about 5.0 mm) and the microneedle array 1200 are shown in FIG. 12B.

One consideration for determining configuration of a microneedle array may be overall signal level provided by the microneedles. Generally, signal level at each microneedle is invariant of the total number of microneedle elements in an array. However, signal levels can be enhanced by electrically interconnecting multiple microneedles together in an array. For example, an array with a large number of electrically connected microneedles is expected to produce a greater signal intensity (and hence increased accuracy) than one with fewer microneedles. However, a higher number of microneedles on a die will increase die cost (given a constant pitch) and will also require greater force and/or velocity to insert into skin. In contrast, a lower number of microneedles on a die may reduce die cost and enable insertion into the skin with reduced application force and/or velocity. Furthermore, in some variations a lower number of microneedles on a die may reduce the overall footprint area of the die, which may lead to less unwanted localized edema and/or erythema. Accordingly, in some variations, a balance among these factors may be achieved with a microneedle array including 37 microneedles as shown in FIGS. 12A-12B or a microneedle array including seven microneedles as shown in FIGS. 11A-11C. However, in other variations there may be fewer microneedles in an array (e.g., between about 5 and about 35, between about 5 and about 30, between about 5 and about 25, between about 5 and about 20, between about 5 and about 15, between about 5 and about 100, between about 10 and about 30, between about 15 and about 25, etc.) or more microneedles in an array (e.g., more than 37, more than 40, more than 45, etc.).

Additionally, in some variations only a subset of the microneedles in a microneedle array may be active during operation of the analyte monitoring device. For example, a portion of the microneedles in a microneedle array may be inactive (e.g., no signals read from electrodes of inactive microneedles). In some variations, a portion of the microneedles in a microneedle array may be activated at a certain time during operation and remain active for the remainder of the operating lifetime of the device. Furthermore, in some variations, a portion of the microneedles in a microneedle array may additionally or alternatively be deactivated at a certain time during operation and remain inactive for the remainder of the operating lifetime of the device.

In considering characteristics of a die for a microneedle array, die size is a function of the number of microneedles in the microneedle array and the pitch of the microneedles. Manufacturing costs are also a consideration, as a smaller die size will contribute to lower cost since the number of dies that can be formed from a single wafer of a given area will increase. Furthermore, a smaller die size will also be less susceptible to brittle fracture due to the relative fragility of the substrate.

As described above, each microneedle in the microneedle array may include an electrode. In some variations, multiple distinct types of electrodes may be included among the microneedles in the microneedle array. For example, in some variations the microneedle array may function as an electrochemical cell operable in an electrolytic manner with three types of electrodes. In other words, the microneedle array may include at least one working electrode, at least one counter electrode, and at least one reference electrode. Thus, the microneedle array may include three distinct electrode types, though one or more of each electrode type may form a complete system (e.g., the system might include multiple distinct working electrodes). Furthermore, multiple distinct microneedles may be electrically joined to form an effective electrode type (e.g., a single working electrode may be formed from two or more connected microneedles with working electrode sites). Each of these electrode types may include a metallization layer and may include one or more coatings or layers over the metallization layer that help facilitate the function of the particular electrode.

Generally, the working electrode is the electrode at which an oxidation reaction and/or a reduction reaction of interest occurs for detection of an analyte of interest. The counter electrode functions to source (provide) or sink (accumulate) the electrons, via an electrical current, that are required to sustain the electrochemical reaction at the working electrode. The reference electrode functions to provide a reference potential for the system; that is, the electrical potential at which the working electrode is biased is referenced to the reference electrode. A fixed, time-varying, or at least controlled potential relationship is established between the working and reference electrodes, and within practical limits no current is sourced from or sinked to the reference electrode. Additionally, to implement such a three-electrode system, the analyte monitoring device may include a suitable potentiostat or electrochemical analog front end to maintain a fixed potential relationship between the working electrode and reference electrode contingents within the electrochemical system (via an electronic feedback mechanism), while permitting the counter electrode to dynamically swing to potentials required to sustain the redox reaction of interest.

Furthermore, the microneedle arrays described herein may have a high degree of configurability concerning where the working electrode(s), counter electrode(s), and reference electrode(s) are located within the microneedle array. This configurability may be facilitated by the electronics system. Microneedle configurations may include different numbers and/or distributions of working electrodes, counter electrodes, and reference electrodes, and different numbers and/or distributions of active electrodes and inactive electrodes.

FIG. 13 depicts a block diagram representation of the sensor assembly 350 of the analyte monitoring device 110. The sensor assembly 350 includes aspects of the microneedle array assembly 360 and the electronics assembly 370, which may include aspects of the electronics system 120 shown in and described with reference to FIG. 2A. While FIG. 13 depicts the sensors (e.g., kinetic sensor 1312, ambient light sensor 1308, etc.) as components of the sensor assembly 350 of the analyte monitoring device 110, it should be understood that in some variations, one or more of these sensors may be located on one or more separate monitoring devices. For example, a system may comprise a first wearable device including a kinetic sensor 1312 and a second, different wearable device including an ambient light sensor 1308. In such variations, the analyte monitoring device 110 may or may not include these sensors, or may include a different subset of sensors than those illustrated in FIG. 13.

The sensor assembly 350 includes the first PCB 450 for the electronics assembly 370 and the second PCB 420 (and optionally the intermediate PCB 425) for the microneedle array assembly 360. The first PCB 450 and the second PCB 420 may be connected, thereby establishing a connection between the microneedle array assembly 360 and the electronics assembly 370, by way of connectors. For example, the second PCB connector 430 on the back side of the second PCB 420 connects to the first PCB connector 470 on the top side of the first PCB 450.

The second PCB connector 430 and the first PCB connector 470 may be electromechanical connectors that provide for communicative coupling between the second PCB 420 and the first PCB 450 to allow for signal communication between the second PCB 420 and the first PCB 450. For example, signals from the microneedle array 140 may be communicated to the first PCB 450 through the second PCB 420, the second PCB connector 430, and the first PCB connector 470.

The first PCB 450 may include various electronic components to receive and process the electrochemical signals received from the microneedle array 140, and some electronic components may be included for additional functionality. As shown in FIG. 13, the first PCB 450 may include coupled thereto an analog front end 1302, a thermistor 1304, a real time clock 1306, an ambient light sensor 1308, a microcontroller 1310 (or a controller), a kinetic sensor 1312, a communication controller 1314, an antenna 1316, the battery 460, a voltage regulator 1322, and a boost circuit 1320. In some variations, the second PCB 420 or the intermediate PCB 425 may include, in addition to the microneedle array 140 and the second PCB connector 430, the thermistor 1304. In some variations, the thermistor 1304 is positioned to minimize its distance to the skin of a user. In some variations, the thermistor 1304 is not included and temperature measurements may be obtained from a temperature sensor that is incorporated in the analog front end 1302. In some variations, fewer, additional, and/or alternative components may be included in the sensor assembly 350, as described further herein.

The analog front end 1302 is part of the sensor assembly 350 (e.g., part of the electronics assembly 370) of the analyte monitoring device 110. The analog front end 1302 may include sensor circuitry (e.g., sensor circuitry 124 as shown in FIG. 2A) that converts analog current measurements to digital values that can be processed by the microcontroller 1310. The analog front end 1302 may, for example, include a programmable analog front end that is suitable for use with electrochemical sensors. In some variations, the analog front end 1302 may be an ultra-low power programmable analog front end for use with electrochemical sensors. In some variations, the analog front end 1302 may be a high precision, impedance, and electrochemical front end. In some variations, the analog front end 1302 may be a configurable analog front end potentiostat for low-power chemical sensing applications. The analog front end 1302 may provide biasing and a complete measurement path, including the analog to digital converters (ADCs). Ultra-low power may allow for the continuous biasing of the microneedle array 140 to maintain accuracy and fast response.

In some variations, the analog front end 1302 may be compatible with both two and three terminal electrochemical sensors, such as to enable both DC current measurement, AC current measurement, and electrochemical impedance spectroscopy (EIS) measurement capabilities. Furthermore, the analog front end 1302 may include an internal temperature sensor and programmable voltage reference, support external temperature monitoring, provide an external reference source, and integrate voltage monitoring of bias and supply voltages for safety and compliance.

In some variations, the analog front end 1302 may include a multi-channel potentiostat to multiplex sensor inputs and handle multiple signal channels. For example, the analog front end may include a multi-channel potentiostat such as that described in U.S. Pat. No. 9,933,387, which is incorporated herein in its entirety by this reference.

In some variations, the analog front end 502 and peripheral electronics may be integrated into an application-specific integrated circuit (ASIC). In some variations, this integrated solution may include the microcontroller 1310 described below.

In some variations, the sensor assembly 350 of the analyte monitoring device 110 may include at least one microcontroller 1310 (e.g., the controller 122 shown in FIG. 2A) incorporated in the electronics assembly 370. The microcontroller 1310 may include, for example, a processor with integrated flash memory. In some variations, the microcontroller 1310 in the analyte monitoring device 110 may be configured to perform analysis to correlate sensor signals to an analyte measurement (e.g., glucose measurement). For example, the microcontroller 1310 may execute a programmed routine in firmware to interpret the digital signal (e.g., from the analog front end 1302), perform any relevant algorithms and/or other analysis, and route processed data to and/or from a communication module (e.g., the communication module 126 shown in FIG. 2A). Keeping the analysis on-board the analyte monitoring device 110 may, for example, enable the analyte monitoring device 110 to broadcast analyte measurement(s) to multiple devices (e.g., mobile computing devices such as a smartphone or smartwatch, therapeutic delivery systems such as insulin pens or pumps, etc.) in parallel, while ensuring that each connected device has the same information.

In some variations, the microcontroller 1310 may be configured to activate and/or inactivate the analyte monitoring device 110 in response to one or more detected conditions or states of one or more of the environment (e.g., surrounding area of the analyte monitoring device 110) or components of the analyte monitoring device 110. For example, the microcontroller 1310 may be configured to power on the analyte monitoring device 110 in response to one or more conditions, such as insertion of the microneedle array 140 into skin, removal of the analyte monitoring device 110 from an applicator device, transition of the analyte monitoring device 110 from an unusable state to a usable state, and a command from an external device, as further described herein. The microcontroller 1310 may be configured to power on the analyte monitoring device 110 in response to a determination of a valid power-on event. Based on the type of valid power-on event, the microcontroller 1310 may transition the analyte monitoring device 110 to a corresponding mode of operation. In some variations, the microcontroller 1310 may be configured to determine a source of a power-on event and transition the analyte monitoring device 110 to a mode of operation corresponding to the source of the power-on event.

In some variations, the microcontroller 1310 may utilize an 8-bit, 16-bit, 32-bit, or 64-bit data structure. Suitable microcontroller architectures include Reduced Instruction Set Computer (RISC) architectures or Complex Instruction Set Computer (CISC) architectures, and flash memory may be embedded or external to the microcontroller 510 for suitable data storage. In some variations, the microcontroller 1310 may be a single core microcontroller, while in some variations the microcontroller 1310 may be a multi-core (e.g., dual core) microcontroller which may enable flexible architectures for optimizing power and/or performance within the analyte monitoring device 110. For example, the cores in the microcontroller 1310 may include similar or differing architectures. For example, in an example variation, the microcontroller 1310 may be a dual core microcontroller including a first core with a high performance and high-power architecture, and a second core with a low performance and low power architecture. The first core may function as a “workhorse” in that it may be used to process higher performance functions (e.g., sensor measurements, algorithmic calculations, etc.), while the second core may be used to perform lower performance functions (e.g., background routines, data transmission, etc.). Accordingly, the different cores of the microcontroller 510 may be run at different duty cycles (e.g., the second core for lower performance functions may be run at a higher duty cycles) optimized for their respective functions, thereby improving overall power efficiency. In some variations, the microcontroller 1310 may include embedded analog circuitry, such as for interfacing with additional sensors and/or the microneedle array 140. In some variations, the microcontroller 510 may be configured to operate using a 0.8V to 5V power source, such as a 1.2V to 3V power source.

In some variations, the sensor assembly 350 of the analyte monitoring device 110 may include at least one communication module (e.g., communication module 126 as shown in FIG. 2A), such as a wireless communication module to communicate with one or more devices. For example, the communication module may include a wireless transceiver that is integrated into the microcontroller 1310. However, the electronics assembly 370 may additionally or alternatively include a communication module that is separate from the microcontroller 1310. In some variations, the communication module may communicate via wireless network (e.g., through Bluetooth, NFC, WiFi, RFID, Thread, 6LoWPAN, LoRa, or any type of data transmission that is not connected by cables). For example, devices may directly communicate with each other in pairwise connection (1:1 relationship, e.g., unicasting) or in a hub-spoke or broadcasting connection (“one to many” or 1: m relationship, e.g., multicasting). As another example, the devices may communicate with each other through mesh networking connections (e.g., “many to many”, or m:m relationships, or ad-hoc), such as through Bluetooth mesh networking. Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), or any other suitable communication protocol. Some wireless network deployments may combine networks from multiple cellular networks or use a mix of cellular, Wi-Fi, and satellite communication. In an example variation, the communication module may include a wireless transceiver integrated into the microcontroller and including a Bluetooth Low Energy compatible radio that complies with the Bluetooth Special Interest Group 5.0 specification.

The communication module may further include or be coupled to one or more antennas (e.g., antenna 128 as shown in FIG. 2A). For example, the sensor assembly 350 may include a chip antenna mounted on the first PCB 450 or an antenna implemented directly onto the first PCB 450, which may provide better range while reducing cost and complexity. In some variations, as shown in FIG. 13, a communication controller 1314 may be part of the sensor assembly 350 on the first PCB 450 and may be coupled to an antenna 1316 for wireless communication according to various wireless protocols as described above. In a variation, the communication controller 1314 is an NFC tag IC or NFC module. In some variations, rather than occupy space on the first PCB 450, the antenna may be contained in or etched on the housing, such as on an underside area or topside area of the housing cover 320. For example, a portion of the housing cover 320 may be metallicized and metal may be deposited to form the antenna, with contacts between the metal and the first PCB 450 incorporated. In other variations, a flexible PCB may be incorporated for the antenna and fitted, for example, within the housing cover 320, and contacts between the flexible PCB and the main PCB may be incorporated. By incorporating the antenna into a space separate from the first PCB 450, additional space is made available on the first PCB 450 and increased performance may be achieved by optimizing the placement of the antenna (e.g., at a point closest to or on the housing cover 320).

In some variations, the microcontroller 1310 on the analyte monitoring device 110 may have a unique serial number, enabling tracking of the analyte monitoring device 110 during production and/or field use.

In some variations, remote devices can come in and out of range from the communication controller 1314 (or other communication module) to connect and reconnect so that the user is able to seamlessly connect and transfer information between devices. For example, a monitoring device, such as, for example, the analyte monitoring device 110, may be communicably coupled with one or more remote devices configured to receive, process, and analyze one or more data types monitored from the analyte monitoring device 110. As is discussed in detail below with respect to the activity and inactivity detection models, one or more additional monitoring devices (which may be wearable), may additionally or alternatively be communicably coupled to one or more remote devices.

As described above, in some variations, the analyte monitoring device 110 may include one or more sensors in addition to the microneedle array 140. For example, the analyte monitoring device 110 may include one or more temperature sensors configured to measure skin temperature, which may be used to enable temperature compensation for the microneedle array 140. For example, in some variations, a thermistor 1304 (or other temperature sensor such as a resistance temperature detector, a semiconductor junction, a bimetallic sensor, and a thermopile sensor) may be coupled to the first PCB 450 or the second PCB 420. The thermistor 1304 may be arranged near a skin-facing portion or outer facing side of the sensor assembly, such as on the second PCB 420 or on the top side of the first PCB 450 (e.g., near or adjacent to the back plate 330). The back plate 330 may be of a suitable thickness to reduce thermal resistance and improve heat transfer and measurement accuracy.

A sensor may be incorporated in the sensor assembly 350 to enable dynamic adjustment of light levels in indicator lights, such as light emitting diodes (LEDs) 1318, to compensate for environmental light conditions and to help conserve power. The ambient light sensor 1308 may, in some variations, be coupled to the first PCB 450 to sense the level of ambient light in the surrounding environment of the analyte monitoring device 110. Additional details related to utilization of the ambient light sensor 1308 are provided below.

The LEDs 1318 may be coupled to the first PCB 450 of the sensor assembly 350 as part of the electronics assembly 370. The LEDs 1318 may be controlled in one or more predetermined illumination patterns or modes to communicate different statuses and/or other suitable information. An indicator light may be controlled to illuminate with multiple colors (e.g., red, orange, yellow, green, blue, and/or purple, etc.) or in only one color. For example, an indicator light may include a multi-colored LED. As another example, an indicator light may include a transparent or semi-transparent material (e.g., acrylic) positioned over one or more different-colored light sources (e.g., LED) such that different-colored light sources may be selectively activated to illuminate the indicator light in a selected color. The activation of light sources can either occur simultaneously or in sequence. An indicator light may have any suitable form (e.g., raised, flush, recessed, etc. from housing body) and/or shape (e.g., circle or other polygon, ring, elongated strip, etc.). In some variations, an indicator light may have a pinhole size and/or shape to present the same intensity of the light as a larger light source, but with significantly less power requirements, which may help conserve onboard power in the analyte monitoring device 110.

Although LEDs are shown in FIG. 13, in some variations, other types of indicator lights may be incorporated in the sensor assembly 350. For example, the indicator lights may include LEDs, OLEDs, lasers, electroluminescent material, or other suitable light sources or waveguides. In some variations, rather than include LEDs or indicator lights, the dome surface may be a liquid crystal display (LCD) or an E-ink display.

In some variations, additional sensors may be incorporated in the sensor assembly 350. For example, a kinetic sensor 1312 (e.g., as described in further detail below) may be incorporated in the electronics assembly 370 and coupled to the first PCB 450. The kinetic sensor 1312 may be used to further determine appropriate periods for the analyte monitoring device 110 to transition to a power saving mode or a reduced power state. For example, detection of darkness via the ambient light sensor 1308 and no motion of the analyte monitoring device 110 via the kinetic sensor 1312 may indicate that the wearer of the analyte monitoring device 110 is asleep or in a relaxed state, which may trigger the analyte monitoring device 110 to transition to a power saving mode or a reduced power state. In some variations, the kinetic sensor 1312 may be used to track movement of the user of the analyte monitoring device 110 for other purposes. The kinetic sensor 1312 may, for example, include an accelerometer, a gyroscope, and/or an inertial measurement unit to capture positional, displacement, trajectory, velocity, acceleration, and/or device orientation values. For example, such measurements may be used to infer the wearer's physical activity (e.g., steps, intense exercise) over a finite duration. In some variations, the kinetic sensor 1312 may be employed to enable detection of wearer interactions with the analyte monitoring device 110, such as touch or tapping. For example, touch or tap detection can be employed to silence or snooze notifications, alerts, and alarms, control a wirelessly connected mobile computing device, or to activate and/or deactivate a user interface on the analyte monitoring device 110 (e.g., an embedded display or indicator light such as the LEDs 1318). Touching or tapping may be performed in a defined sequence and/or for a predetermined duration (e.g., at least 3 seconds, at least 5 seconds) to elicit certain actions (e.g., display or indicator light deactivation and/or activation). In some variations, the analyte monitoring device 110 may transition to a power saving mode upon detection of limited motion or activity (e.g., absence of significant acceleration) for at least a predetermined period of time (e.g., 15 minutes, 30 minutes, 45 minutes, 1 hour, or other suitable of time), as measured by the kinetic sensor 1312 and/or other sensors.

In some variations, the analyte monitoring device 110 may include at least one real-time clock (RTC) 1306. For example, the real-time clock 1306 may be part of the electronics assembly 370 coupled to the first PCB 450. In some variations, the real-time clock 1306 has an embedded quartz crystal or the like for maintaining an accurate tracking of time. The real-time clock 1306 may be employed to track absolute time (e.g., Coordinated Universal Time, UTC, or local time) when the analyte monitoring device 110 is in storage or during use. In some variations, synchronization to absolute time may be performed following manufacturing of the analyte monitoring device 110. During operation, the real-time clock 1306 may output a clocking signal to the microcontroller 1310 to drive and/or adjust internal clocks of the microcontroller 1310 to ensure that the microcontroller 1310, which may have lower timing accuracy than the real-time clock 1306, is properly tracking time. In some variations, the clocking signal from the real-time clock 1306 to the microcontroller 1310 is a constant signal. In some variations, the clocking signal is sent periodically at predefined intervals.

The real-time clock 1306 may be employed to time-stamp analyte measurements (e.g., glucose measurements) during operation of the analyte monitoring device 110 to create a time-series data set that is communicated to a connected remote device (e.g., mobile computing device), cloud storage, or other suitable data storage device, such as for later review by the user (e.g., wearer of the analyte monitoring device), a support network, a healthcare provider, etc. In some variations, the microcontroller 1310 performs the time-stamping operations.

As shown in FIG. 2A, the analyte monitoring device may include one or more power sources 130 configured to provide power to other components. For example, the analyte monitoring device 110 may include a battery 460. The battery 460 may be any suitable type of battery able to provide power to the various components of the sensor assembly 350. The battery 460 may be a silver-oxide battery, which has a high energy density and is more environmentally friendly than lithium batteries. In some variations, a primary (e.g., non-rechargeable) battery may be used. Furthermore, in some variations, a secondary (e.g., rechargeable) battery may be used. However, any suitable power source may be used, including a lithium-based battery.

In some variations, the battery 460 may be coupled to a boost circuit 1320. The boost circuit 1320 may be part of the electronics assembly 370 coupled to the first PCB 450. The boost circuit 1320 may be incorporated to provide appropriate power levels to certain electronic components. For example, the boost circuit 1320 may boost or increase the voltage provided by the battery 460 to provide sufficient power to one or more of the components of the sensor assembly 350, as further described herein. In some variations, a voltage regulator 1322 may be coupled to an output of the boost circuit 1320 to reduce the noise generated by the boost circuit 1320.

In some variations, the analyte monitoring device 110 may be paired to at least one remote device such that the remote device receives broadcasted or otherwise transmitted data from the analyte monitoring device 110, including measurement data. Suitable remote devices include, for example a mobile computing device (e.g., smartphone, smartwatch, or cloud-based server) which may be executing a mobile application.

As described above, the pairing may be accomplished through suitable wireless communication modules (e.g., NFC and/or Bluetooth). In some variations, the pairing may occur after the analyte monitoring device 110 is applied and inserted into the skin of a user (e.g., after the analyte monitoring device 110 is activated). The pairing may occur prior to the analyte monitoring device 110 being applied and inserted into the skin of a user.

Thus, the paired mobile or other device may receive the broadcasted or transmitted data from the analyte monitoring device 110. The remote device 110 may display, store, and/or transmit the measurement data to the user and/or a healthcare provider and/or a support network. Furthermore, in some variations, the paired mobile or wearable device may perform algorithmic treatment to the data to improve the signal fidelity, accuracy, and/or calibration, etc. In some variations, measurement data and/or other user information may additionally or alternatively be communicated and/or stored via a network (e.g., a cloud network).

By way of illustration, in some variations, a mobile computing device or other computing device (e.g., smartphones, smartwatches, tablets, etc.) may be configured to execute a mobile application that provides an interface to display estimated glucose values, trend information, and historical data, etc. Although the below description refers specifically to glucose as a target analyte, the features and processes described below with respect to glucose may be similarly applied to applications relating to other kinds of analytes.

In some variations, the mobile application may use the mobile computing device's wireless communication framework to scan for the analyte monitoring device 110. The analyte monitoring device 110 may power on or initialize once it is applied to the skin, and the analyte monitoring device 110 may begin an advertising process. The mobile application may then connect to the analyte monitoring device 110 and begin priming the microneedle array 140 for measurement. In case the mobile application detects multiple analyte monitoring devices, the mobile application may detect the analyte monitoring device 110 that is closest in proximity to itself, may request the user (e.g., via the user interface on the mobile device) to confirm disambiguation, and/or may request a confirmation via a physical interaction with the analyte monitoring device 110 that is intended for use (e.g., tapping or other prescribed action with the analyte monitoring device 110 by the user). In some variations, the mobile application may also be capable of connecting to multiple analyte monitoring devices simultaneously. This may be useful, for example, to replace sensors that are reaching the end of their lifetime.

In some variations, the Bluetooth® Low Energy™ (BLE) protocol may be used for connectivity. For example, the sensor implements a custom BLE peripheral profile for the analyte monitoring system. Data may be exchanged after establishing a standard secure BLE connection between the analyte monitoring device and the smartphone, smartwatch, or tablet running the mobile application. The BLE connection may be maintained permanently for the life of the sensor. If the connection is broken due to any reasons (e.g., weak signal) the analyte monitoring device may start advertising itself again and the mobile application may re-establish the connection at the earliest opportunity (e.g., when in range/physical proximity).

In some variations, there may be one or more additional layers of security implemented on top of the BLE connection to ensure authorized access consisting of a combination of one or more techniques such as passcode-protection, shared-secrets, encryption, and multi-factor authentication.

The mobile application may guide the user through initiating a new analyte monitoring device. Once this process completes, the mobile application is not be required for the analyte monitoring device 110 to operate and record measurements. In some variations, a smart insulin delivery device that is connected to the analyte monitoring device 110 can be authorized from the mobile application to receive glucose readings from the sensor directly. In some variations, a secondary display device like a smartwatch can be authorized from the mobile application to receive glucose readings from the sensor directly.

Furthermore, in some variations the mobile application may additionally or alternatively help calibrate the analyte monitoring device 110. For example, the analyte monitoring device 110 may indicate a request for calibration to the mobile application, and the mobile application may request calibration input from the user to calibrate the sensor.

In some variations, a wearable analyte monitoring device, in addition to measuring levels of one or more analytes, may also be utilized to implement a model to determine inactivity states (e.g., rest, such as sleep) of a user. In some variations, an inactivity detection model (e.g., algorithm or state machine) may utilize a neural network, taking inputs from the wearable analyte monitoring device's kinetic sensor 1312 (e.g., an accelerometer) and ambient light sensor 1308. The neural network outputs may be passed through a threshold-based algorithm to filter out brief episodes of wearable signals that resemble, but are not, rest (e.g., sleep).

In some implementations, signals from at least one of the ambient light sensor 1308, the kinetic sensor 1312, and the real time clock 1306 may be used to determine a state of the user, for example, an inactivity state (e.g., a state or rest or sleep). For example, the microcontroller 1310 may implement one or more routines, using as inputs signals from the ambient light sensor 1308, the kinetic sensor 1312, and/or the real time clock 1306, to determine a state of the user. Additionally, a mobile application executed on a mobile computing device may provide an interface that includes details and data related to the determined state of the user. Described herein are exemplary user interfaces that incorporate the display of rest data as determined by the wearable analyte monitoring device.

In some variations, a machine learning model is trained with reference data provided by a plurality of users and is used to determine a likelihood of rest, which in some instances may be classified as sleep. For example, to train the machine learning model, the plurality of users provide records (e.g., written accounts) of periods of time at which they are at rest. Data from the ambient light sensor 1308, the kinetic sensor 1312, and/or the real time clock 1306 is correlated with the user-provided records to formulate a model that predicts if the user is at a state of rest (or sleep) based on input data from the ambient light sensor 1308, the kinetic sensor 1312, and/or the real time clock 1306. Using the received data from the wearable analyte monitoring device, the model may generate a probability that the user is at a state of rest. If the probability that the user is at a state of rest is greater than a defined threshold, then the microcontroller 1310, executing the trained model, may indicate that the user is in a state of rest. If the probability that the user is at a state of rest is less than a defined threshold, then the microcontroller 1310, executing the trained model, may indicate that the user is not in a state of rest.

In some variations, the model is a neural network. In some variations, the model is a multi-layer neural network.

In some variations, a user of the analyte monitoring device may be prompted (e.g., via the interface provided by the mobile app) to provide the times (e.g., one or more periods or ranges of time) at which the user is typically resting (e.g., sleeping). The model may use the inputted typical times of rest with the data from the real time clock 1306 as an input into the model to generate the probability that the user is at a state of rest.

A wearable analyte monitoring device, in some variations, may also be utilized to determine activity states of a user, such as a state of physical exercise. In some variations, activity detection may rely on a wearable device (e.g., analyte monitoring or other monitoring device's) kinetic sensor 1312 (e.g., an accelerometer) and a state machine-based algorithm. When the user is deemed inactive, recent accelerometer data may be analyzed to determine whether physical activity may have started. While the user is in the middle of physical activity, data from the kinetic sensor 1312 may be checked (e.g., continuously or at predetermined intervals) to determine whether physical activity has ended.

In some variations, the kinetic sensor 1312 may count a number of steps every five minutes, although other time periods may be used. If there is a minimum threshold number of steps within the time period, the step count may be recorded. This may help reduce memory usage and may also provide for quick data transfer. In some variations, step counts are converted into periods of sedentary, low, moderate, or high activity. Each step count category is representative of a range of step counts. In some variations, low, moderate, and high periods of activity are combined to result in active periods of time. In other variations, different categories may be defined and used to represent active periods of time.

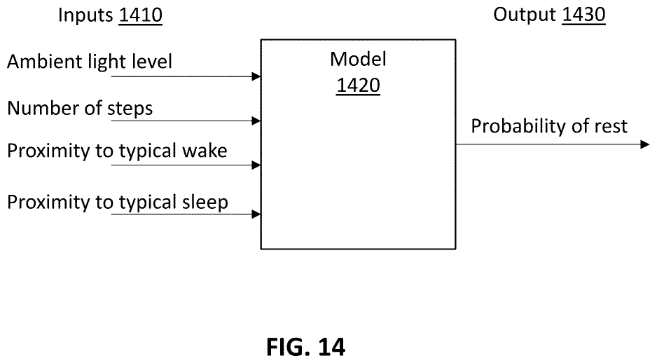

In some variations, an analyte monitoring device may comprise one or more sensors configured for activity and/or inactivity detection. FIG. 14 depicts a system block diagram of an inactivity detection model in a wearable analyte monitoring device. In some variations, the inactivity detection may comprise sleep detection. Signal inputs 1410 from the wearable analyte monitoring device to a model 1420 include ambient light level from the ambient light sensor 1308, number of steps from the kinetic sensor 1312, and/or time data from the real time clock 1306, which may indicate the proximity to the typical wake time of the user and the proximity to the typical sleep time of the user. In some variations, the model 1420 may be a trained model. The ambient light level may include real-time data over a predetermined period of time, and the step data may also be real-time data over a predetermined period of time. The model 1420, having been trained with historical user data that indicates values of the input signals at which the user is typically at rest or sleeping, determines a probability that the user is at rest.

In some variations, if the probability that the user is at rest is above a threshold value for a threshold period of time, the model 1420 determines that the user is at rest. If the probability that the user is at rest is not above a threshold value or is not above the threshold value for the threshold period of time, the model 1420 determines that the user is not at rest. The output 1430 of the model 1620 may comprise a probability of rest.

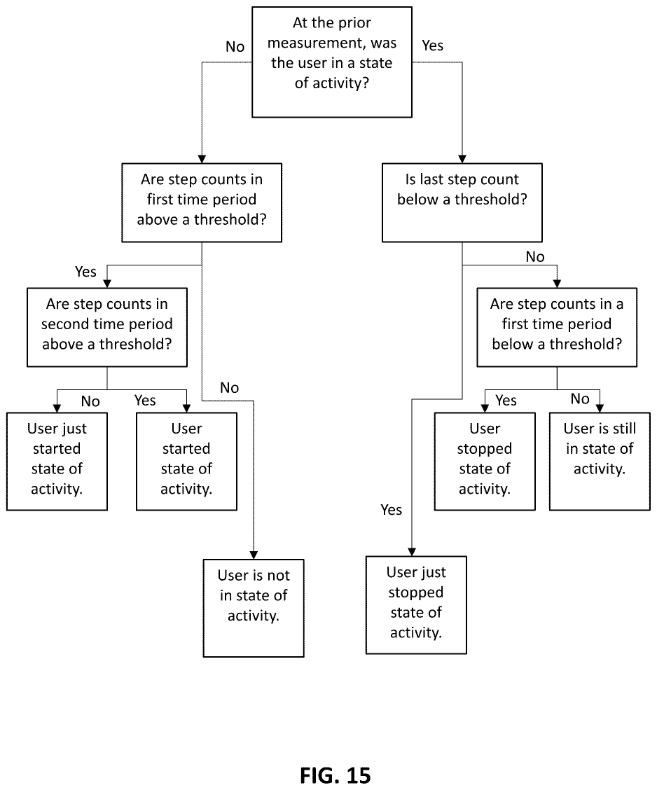

FIG. 15 depicts a block diagram of an exemplary activity detection process that may be implemented in an analyte monitoring device. The process begins with a determination of, at the previous measurement, if the user was in an activity state (e.g., a period of activity like exercise). If the user was not in an activity state, a determination is made as to whether a step count within a first predetermined time period (e.g., 10 minutes) is above a first threshold step count. If not, the user is determined to not be in an activity state. If yes, a second predetermined time period is analyzed to determine if the number of the step count is above a second threshold step count. If no, it is determined that the user has just begun an activity state. If yes, it is determined that the user started an activity state within the second predetermined time period. In some variations, the first predetermined time period is greater than the second predetermined time period. In some variations, the first threshold step count is greater than the second threshold step count.

Further, referring to FIG. 15, if the user was initially determined to be in a an activity state, a determination is made as to whether the activity state has ended. This may be achieved by evaluating if the last step count is below a threshold step count. If yes, it is be determined that the activity state ended. If no, the step count over the first predetermined time period is compared to the threshold time count. If yes, the user is no longer in an activity state. If not, the user is still in an activity state.

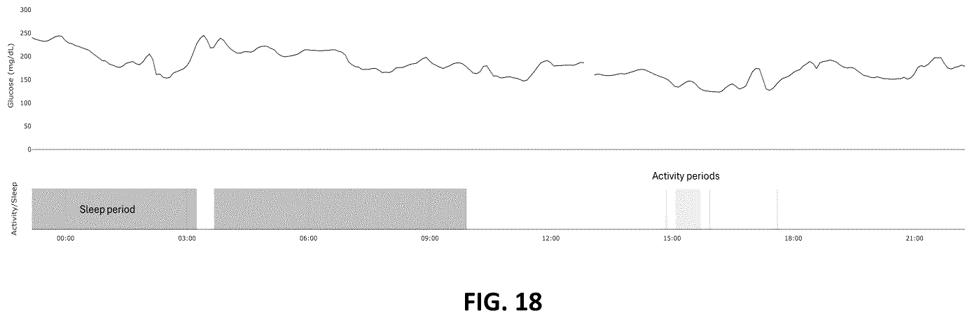

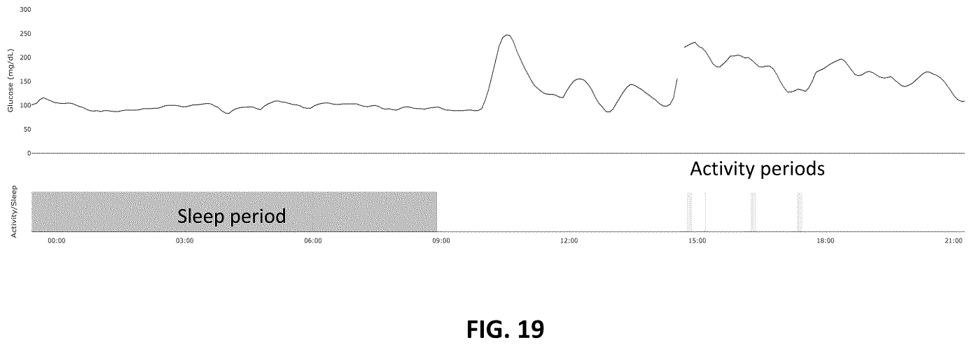

FIG. 18 provides a first example plot of glucose, physical activity, and sleep, each of which are determined by the analyte monitoring system that is adapted to determine a state of a user, including activity (physical exercise) states and inactivity (rest) states. FIG. 19 similarly provides a second example plot of glucose, physical activity, and sleep, each of which are determined by the analyte monitoring system that is adapted to determine a state of a user, including activity (physical exercise) states and inactivity (rest) states.

Models for activity and inactivity detection are described in further detail below. As used herein, a “model” may refer to a functional component configured to perform one or more tasks, such as receiving input data, processing the data, and generating output data. A model may comprise an algorithm, a state machine, or a combination thereof. A model may refer to a specific computational structure, such as a predictive model, a machine learning model, a neural network, or a rule-based algorithm, that processes input data to generate an output. The model may be implemented as software instructions stored on a non-transitory computer-readable medium. When executed by one or more processors of a remote device, these instructions may cause the processors to perform the functions of the model. Thus, references to a model performing a function may be understood to mean that a processor executes instructions stored on a non-transitory computer-readable medium to perform that function.

While the previous description focuses on features and capabilities of analyte monitoring devices, the detection models described below may be executed with or without an analyte monitoring device. In some variations, one or more separate monitoring devices may be utilized. Each monitoring device may comprise a sensor configured to monitor a physiological or environmental condition. For example, systems for detecting activity and/or inactivity of a user may comprise at least one of an activity monitoring device (e.g., comprising an accelerometer, gyroscope, and/or an inertial measurement unit), a light monitoring device (e.g., comprising a light sensor), or a temperature monitoring device (e.g., comprising a temperature sensor). In some variations, a monitoring device may be a wearable device comprising a wearable sensor. In some variations, a single monitoring device may be configured to monitor a plurality of conditions. For example, a first monitoring device may be configured to detect both activity (e.g., step count) and light. Alternatively, a first monitoring device may be used to detect activity, while a second, different monitoring device may be used to detect light. Further, as described above, in some variations, the systems herein may comprise a third monitoring device (which could be the same device as the first and/or second monitoring device, or a third, different monitoring device) configured to detect analyte levels, such as the analyte monitoring device 110 described herein throughout. Moreover, one or more monitoring devices may be communicable coupled with each other and/or with a remote device (or a plurality of remote devices) to transmit data therebetween.

### Activity Detection Model

The following description is directed to devices, systems, and methods for detecting activity states of a user by analyzing activity data over evaluation intervals using an activity detection model. Broadly, the activity detection model may be configured to differentiate between periods of exercise and periods of inactivity. This activity detection may be performed in real-time or through post-processing of historical data sets. While the activity detection model may operate in conjunction with an analyte monitoring device-for example, to provide context for fluctuations in analyte (e.g., glucose) levels-the model and the associated methods for identifying activity states may also be implemented independently of any analyte monitoring hardware. In such variations, the system may rely on activity data received from any number of wearable or mobile monitoring devices, or combinations thereof, to determine the onset, duration, and/or end of an activity state.

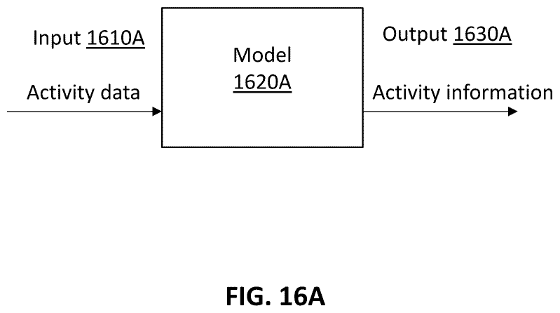

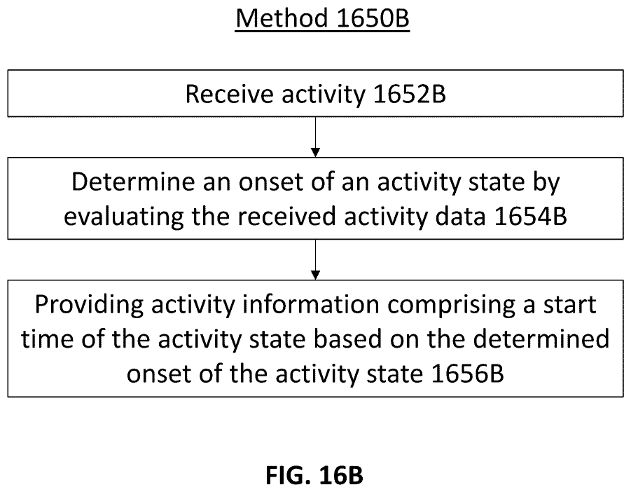

FIG. 16A depicts a block diagram of an activity detection model which may be implemented by a remote device. The activity detection model may comprise a model 1620A, such as an algorithm and/or a state-machine configured to receive an input 1610A and generate an output 1630A. In some variations, the model 1620A may be implemented as a rule-based state machine that transitions between activity states based on threshold comparisons. In some variations, the model 1620A may comprise a predictive model, such as a machine learning model, a neural network (with one or more layers), a random forest, a support vector machine, or a combination thereof. In some variations, the model 1620A may be trained using reference data from a plurality of users, such as historical step counts correlated with user-verified exercise logs or data from reference devices such as fitness trackers. The plurality of users may comprise at least 10, at least 25, at least 50, at least 75, at least 100, at least 250, at least 500, at least 750, or at least 1,000 users. The model 1620A may be executed by a processor within the remote device, which may store the model in a non-transitory computer-readable medium. The remote device may be coupled with one or more monitoring devices (e.g., an activity monitoring device comprising a wearable sensor) that transmit the activity data thereto.

The input 1610A may comprise activity data, which may include any data indicative of physical movement or exertion by the user. For example, the activity data may comprise a step count, a cadence, a distance traveled, and/or raw kinetic data such as acceleration or velocity. This activity data may be received from one or more monitoring devices, such as a wearable device (e.g., comprising a wearable sensor), a smartphone, or a smartwatch. In some variations, the activity data may be provided by a kinetic sensor, such as an accelerometer. In some variations, the activity data may be provided by a kinetic sensor, such as an accelerometer, integrated into an analyte monitoring device (as described above). In such variations, the accelerometer may output processed step count data rather than raw acceleration values, which may conserve memory and processing resources on the wearable device while still providing useful activity indicators. Alternatively, the accelerometer may output raw data, which in some variations may increase accuracy of the step count and downstream activity state determinations.

The activity data may be sampled or recorded over sampling intervals. A sampling interval may define a time period over which activity data (e.g., step counts, raw acceleration values) is accumulated before being stored or transmitted. In some variations, the activity data may comprise raw data (e.g., acceleration values) may be processed on the remote device to determine associated processed data (e.g., step count). In some variations, a sampling interval may be about 30 seconds to about 15 minutes, such as about 1 minutes to about 10 minutes, about 2 minutes to about 9 minutes, about 3 minutes to about 8 minutes, or about 4 minutes to about 7 minutes, about 4.5 minutes to about 6 minutes, or about 5 minutes to about 5.5 minutes. In some variations, the sampling interval may be about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, or more than about 15 minutes. In some variations, the sampling interval may be less than 5 minutes, 5 minutes, or more than 5 minutes. For example, the wearable sensor may record data (e.g., step counts) every 5 minutes, where each recorded value represents a total number of steps detected during that 5-minute period. Furthermore, if no steps are detected during a sampling interval, the device may record a value of zero or may omit the record to conserve storage, in which case the absence of data for a given sampling interval may be interpreted as zero activity.

In some variations, a minimum activity threshold may be applied to filter the activity data before further processing. Activity data that does not meet the minimum activity threshold may be excluded from subsequent analysis or may be treated as indicating an inactive state. For example, sampling intervals having fewer than a threshold number of steps (e.g., fewer than 4 steps, fewer than 10 steps, or fewer than 50 steps) may not be classified as activity data. In some variations, the minimum activity threshold for step count may be about 1 step to about 50 steps, such as about 2 to about 45 steps, about 3 to about 40 steps, about 4 to about 35 steps, about 5 to about 30 steps, about 6 to about 25 steps, about 7 to about 20 steps, about 8 to about 15 steps, about 9 to about 13 steps, or about 10 to about 11 steps. This filtering may reduce noise from incidental movements that do not represent meaningful physical activity. In some variations, sampling intervals having a step count greater than 50 steps may be classified as activity data, while sampling intervals with 50 or fewer steps may not be classified as activity data.

The model 1620A may process or evaluate the received activity data to determine an onset of an activity state, an end of an activity state, or both. To determine an activity state onset or end, the model may evaluate activity data across one or more evaluation intervals. An evaluation interval may define a time period over which received data is aggregated and compared against activity thresholds to analyze the data. In some variations, an evaluation interval may span one or more sampling intervals. The duration of an evaluation interval may be about 1 minute to about 60 minutes, such as about 2 minutes to about 50 minutes, about 3 minutes to about 40 minutes, about 4 minutes to about 30 minutes, about 5 minutes to about 20 minutes, about 6 minutes to about 15 minutes, about 7 minutes to about 12 minutes, or about 8 minutes to about 10 minutes. In some variations, the time period of an evaluation internal may be 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 30 minutes, about 45 minutes, or about 60 minutes (e.g., 5 minutes, 10 minutes, or 15 minutes).

In some variations, the model 1620A may evaluate activity data within a first evaluation interval and within a second evaluation interval. The first and second evaluation intervals may have different durations or the same duration. In some variations, the first and second evaluation intervals may overlap. For example, the second evaluation interval may precede at least a portion of the first evaluation interval. In these variations, the second evaluation interval may overlap with only an earlier portion of the first evaluation interval. Thus, the second evaluation interval may comprise data from a first, earlier portion of the first evaluation interval. In one example, the first evaluation interval may comprise the most recent 10 minutes of activity data (e.g., from two most recent 5-minute sampling intervals), and the second evaluation interval may comprise only the earlier of those two sampling intervals (i.e., the 5-minute period that began 10 minutes ago and ended 5 minutes ago). This temporal relationship may enable the system to determine not only whether activity has started, but also to pinpoint when the activity started with greater precision than would be possible using a single evaluation interval. Specifically, using evaluation intervals of different durations may enable the system to balance sensitivity against specificity in activity detection. For example, a longer evaluation interval (e.g., 10 minutes)—which may be a first evaluation interval—may provide a broader view of activity patterns and help confirm that sustained activity is occurring, while a shorter evaluation interval (e.g., 5 minutes)—which may be a second evaluation interval—may provide finer temporal resolution for pinpointing a start or end time of a user's activity state.

During evaluation, the model 1620A may compare activity data to one or more activity thresholds. An activity threshold may comprise a value representative of a minimum amount of movement required to classify a state as active. In some variations, a step-based activity threshold may be expressed as a step count per evaluation interval, a step count per sampling interval, a step rate, or combinations thereof. For example, a step rate may be expressed in steps per minute (steps/min) calculated by dividing a step count accumulated over an interval by a duration of that interval in minutes. In some variations, a step rate threshold may be about 0.1 steps/min to about 150 steps/min, such as about 0.5 steps/min to about 100 steps/min, about 1 step/min to about 80 steps/min, about 2 steps/min to about 60 steps/min, about 5 steps/min to about 50 steps/min, about 10 steps/min to about 40 steps/min, about 12 steps/min to about 35 steps/min, about 15 steps/min to about 30 steps/min, about 20 steps/min to about 25 steps/min, or about 22 steps/min to about 28 steps/min. In some variations, the step rate threshold may be about 0.5 steps/min, about 1 step/min, about 2 steps/min, about 5 steps/min, about 8 steps/min, about 10 steps/min, about 12 steps/min, about 15 steps/min, about 18 steps/min, about 20 steps/min, about 25 steps/min, about 30 steps/min, about 35 steps/min, about 40 steps/min, about 50 steps/min, or more than about 50 steps/min. For example, an activity threshold may be a step count threshold ranging from 4 steps to 1,000 steps per evaluation interval, such as about 25 steps to about 750 steps, about 30 steps to about 500 steps, about 40 steps to about 350 steps, about 45 steps to about 300 steps, about 50 steps to about 250 steps, about 55 steps to about 200 steps, about 60 steps to about 175 steps, about 70 steps to about 150 steps, about 75 steps to about 125 steps, about 80 steps to about 110 steps, or about 90 steps to about 100 steps. In some variations, an activity threshold for evaluating the step count may be about 4 steps, about 5 steps, about 6 steps, about 7 steps, about 8 steps, about 9 steps, about 10 steps, about 15 steps, about 20 steps, about 25 steps, about 30 steps, about 40 steps, about 45 steps, about 50 steps, about 55 steps, about 60 steps, about 65 steps, about 70 steps, about 75 steps, about 80 steps, about 85 steps, about 90 steps, about 95 steps, about 100 steps, about 150 steps, about 200 steps, about 225 steps, about 250 steps, about 275 steps, about 300 steps, about 325 steps, about 350 steps, about 375 steps, about 400 steps, about 500 steps, or more than 500 steps per evaluation interval. The model may determine an onset of an activity state if activity data in the first evaluation interval satisfies a first threshold and activity data in the second evaluation interval satisfies a second activity threshold (which may be the same or different than the first activity threshold). In some variations, the first activity threshold may be greater than the second activity threshold. Similarly, the model 1620A may determine the end of an activity state if activity data falls below at least a third activity threshold for at least a third evaluation interval, as will be described in more detail herein.

Further, the model may use different activity thresholds for different purposes. For example, a first activity threshold may be used to initially detect that activity may be occurring, while a second activity threshold may be used to refine the determination of when activity started. In some variations, the first activity threshold may be greater than the second activity threshold. Using a higher first activity threshold may ensure that activity detection is triggered only when meaningful activity is occurring, thereby reducing false positive detections from minor movements. The lower second activity threshold may then allow the system to look backward in time to identify a true onset of activity, which may have begun at a lower intensity before ramping up to the level that triggered the initial detection.

For example, the model 1620A may determine an onset of an activity state if activity data in the first evaluation interval satisfies a first activity threshold and activity data in the second evaluation interval satisfies a second, different activity threshold. Additionally, in some variations, the first evaluation interval may be longer than the second evaluation interval.

In some variations, the first evaluation interval may be about 2 minutes to about 60 minutes, such as about 3 minutes to about 45 minutes, about 4 minutes to about 30 minutes, about 5 minutes to about 20 minutes, about 6 minutes to about 15 minutes, about 7 minutes to about 12 minutes, about 8 minutes to about 10 minutes, or about 9 minutes to about 11 minutes. In some variations, the first evaluation interval may be about 5 minutes, about 7.5 minutes, about 10 minutes, about 12 minutes, about 15 minutes, about 20 minutes, about 30 minutes, about 45 minutes, or about 60 minutes. In one example, the first evaluation interval may comprise about 10 minutes.

In some variations, the second evaluation interval may be about 1 minute to about 30 minutes, such as about 2 minutes to about 25 minutes, about 3 minutes to about 20 minutes, about 4 minutes to about 15 minutes, about 4.5 minutes to about 10 minutes, or about 5 minutes to about 7.5 minutes. In some variations, the second evaluation interval may be about 2 minutes, about 3 minutes, about 4 minutes, about 5 minutes, about 6 minutes, about 7.5 minutes, about 8 minutes, about 9 minutes, about 10 minutes, about 12 minutes, about 15 minutes, about 20 minutes, about 25 minutes, or about 30 minutes. In one example, the second evaluation interval may comprise about 5 minutes.

In some variations, a first activity threshold may be applied to activity data aggregated over a first evaluation interval to determine whether an activity onset evaluation is triggered. In some variations, the first activity threshold may be about 50 steps to about 1,500 steps per first evaluation interval, such as about 75 steps to about 1,250 steps, about 100 steps to about 1,000 steps, about 150 steps to about 750 steps, about 200 steps to about 600 steps, about 225 steps to about 500 steps, about 250 steps to about 450 steps, about 275 steps to about 400 steps, about 290 steps to about 350 steps, or about 295 steps to about 325 steps. In some variations, the first activity threshold may be about 50, about 75, about 100, about 125, about 150, about 175, about 200, about 225, about 250, about 275, about 300, about 325, about 350, about 400, about 450, about 500, about 600, about 750, about 1,000, about 1,250, or about 1,500 steps per first evaluation interval.

In some variations, the first activity threshold may correspond to a step rate of about 5 steps/min to about 60 steps/min, such as about 8 steps/min to about 50 steps/min, about 10 steps/min to about 40 steps/min, about 12 steps/min to about 35 steps/min, about 15 steps/min to about 30 steps/min, about 18 steps/min to about 28 steps/min, or about 20 steps/min to about 25 steps/min. In one example, the first activity threshold may comprise about 300 steps over about 10 minutes (i.e., about 30 steps/min).

In some variations, a second activity threshold may be applied to activity data aggregated over a second evaluation interval that is shorter than, and precedes at least a portion of, the first evaluation interval, thereby refining a start time of the activity state. In some variations, the second activity threshold may be about 10 steps to about 500 steps per second evaluation interval, such as about 20 steps to about 400 steps, about 30 steps to about 300 steps, about 40 steps to about 250 steps, about 50 steps to about 200 steps, about 60 steps to about 150 steps, about 65 steps to about 125 steps, about 70 steps to about 110 steps, about 72 steps to about 100 steps, or about 73 steps to about 90 steps. In some variations, the second activity threshold may be about 10 steps, about 15, about 20, about 25, about 30, about 40, about 50, about 60, about 75, about 80, about 90, about 100, about 125, about 150, about 200, about 250, about 300, about 400, or about 500 steps per second evaluation interval.

In some variations, the second activity threshold may correspond to a step rate of about 1 steps/min to about 50 steps/min, such as about 2 steps/min to about 40 steps/min, about 5 steps/min to about 30 steps/min, about 8 steps/min to about 25 steps/min, about 10 steps/min to about 20 steps/min, or about 12 steps/min to about 18 steps/min. In one example, the second activity threshold may comprise about 75 steps over about 5 minutes (i.e., about 15 steps/min).

In one example, the first activity threshold may be about 300 steps over a 10-minute first evaluation interval, and the second activity threshold may be about 75 steps over a 5-minute second evaluation interval, where the second evaluation interval comprises activity data from an earlier portion of the first evaluation interval. That is, if at least 300 steps are detected over the first evaluation interval, the system may evaluate whether at least 75 steps occurred in the earlier 5-minute portion of that interval. If so, the activity onset may be determined to occur at the beginning of the second evaluation interval, indicating that activity onset occurred approximately 5 to 10 minutes prior. If the second activity threshold is not met, but the first activity threshold is met, the activity onset may be determined to occur at a current time, indicating that activity has just begun. Based on this determination, the system may be configured to define a start time of the activity state as a boundary of a most-recent sampling interval. Put another way, the start time may be assigned to an earlier-in-time boundary or a later-in-time boundary of the most-recent sampling interval. This dual-threshold approach with backdating may enable accurate determination of activity start times even when users gradually transition from inactivity to exercise. Because step counter data from accelerometers—particularly in low-power wearable devices—may not correlate well with reference devices at low activity levels (e.g., under about 150 steps), using calibrated activity thresholds may ensure that detected activity periods reflect genuine exercise rather than sensor noise or incidental motion, thereby reducing false positive activity detections.

Similarly, the model 1620A may determine an end of an activity state based on activity data falling below one or more additional activity thresholds. The activity threshold(s) for evaluating the end of the activity state may be different from those used to determined the onset thereof. In some variations, a third activity threshold for determining an end of the activity state may be lower than the first and/or second activity threshold used for onset detection. For example, the third activity threshold may be about 5 steps, about 10 steps, about 15 steps, about 20 steps, 25 steps, about 30 steps, about 40 steps, about 50 steps, about 75 steps, about 100 steps, about 125 steps, or about 150 steps over a third evaluation interval.

In some variations, determining the end of an activity state may involve evaluating activity data over a plurality of evaluation intervals to confirm that activity has ceased rather than paused temporarily. For example, a third activity threshold may be applied to activity data over a third evaluation interval, and if the third activity threshold is not met, a fourth activity threshold may be applied to activity data over a fourth evaluation interval to confirm the end of the activity state. In some variations, the fourth evaluation interval may define a longer time period than the third evaluation interval. Additionally, or alternatively, the fourth activity threshold may be greater than the third activity threshold.

In some variations, the third evaluation interval may be about 30 seconds to about 30 minutes, such as about 1 minute to about 20 minutes, about 2 minutes to about 15 minutes, about 3 minutes to about 12 minutes, about 4 minutes to about 10 minutes, or about 5 minutes to about 7.5 minutes. In some variations, the third evaluation interval may be about 1 minute, about 2 minutes, about 3 minutes, about 4 minutes, about 5 minutes, about 6 minutes, about 7.5 minutes, about 8 minutes, about 9 minutes, about 10 minutes, about 12 minutes, about 15 minutes, about 20 minutes, about 25 minutes, or about 30 minutes.

In some variations, the fourth evaluation interval may be about 1 minute to about 60 minutes, such as about 2 minutes to about 50 minutes, about 3 minutes to about 40 minutes, about 4 minutes to about 30 minutes, about 5 minutes to about 20 minutes, about 6 minutes to about 15 minutes, about 7 minutes to about 12 minutes, or about 8 minutes to about 10 minutes. In some variations, the fourth evaluation interval may be about 5 minutes, about 7.5 minutes, about 10 minutes, about 12 minutes, about 15 minutes, about 20 minutes, about 30 minutes, about 45 minutes, or about 60 minutes.

In some variations, the third activity threshold may be about 0 steps to about 300 steps per third evaluation interval, such as about 1 step to about 250 steps, about 5 steps to about 200 steps, about 10 steps to about 150 steps, about 15 steps to about 125 steps, about 20 steps to about 100 steps, about 25 steps to about 90 steps, about 30 steps to about 80 steps, about 35 steps to about 75 steps, about 40 steps to about 70 steps, about 45 steps to about 65 steps, or about 48 steps to about 55 steps. In some variations, the third activity threshold may be about 0, about 1, about 2, about 3, about 4, about 5, about 10, about 15, about 20, about 25, about 30, about 40, about 50, about 60, about 75, about 80, about 90, about 100, about 125, about 150, about 200, about 250, or about 300 steps per third evaluation interval.

In some variations, the third activity threshold may correspond to a step rate of about 0 steps/min to about 30 steps/min, such as about 0.5 steps/min to about 25 steps/min, about 1 steps/min to about 20 steps/min, about 2 steps/min to about 15 steps/min, about 5 steps/min to about 12 steps/min, or about 8 steps/min to about 10 steps/min.

n some variations, a fourth activity threshold may be applied over a fourth evaluation interval that is longer than the third evaluation interval to confirm that activity has ended. In some variations, the fourth activity threshold may be about 10 steps to about 1,000 steps per fourth evaluation interval, such as about 25 steps to about 750 steps, about 50 steps to about 500 steps, about 75 steps to about 400 steps, about 100 steps to about 300 steps, about 125 steps to about 250 steps, about 140 steps to about 200 steps, about 145 steps to about 175 steps, or about 148 steps to about 160 steps. In some variations, the fourth activity threshold may be about 10, about 25, about 50, about 75, about 100, about 125, about 150, about 175, about 200, about 250, about 300, about 400, about 500, about 750, or about 1,000 steps per fourth evaluation interval.

In some variations, the fourth activity threshold may correspond to a step rate of about 0.5 steps/min to about 60 steps/min, such as about 2 steps/min to about 50 steps/min, about 5 steps/min to about 40 steps/min, about 8 steps/min to about 30 steps/min, about 10 steps/min to about 20 steps/min, or about 12 steps/min to about 18 steps/min.

In one example, the third evaluation interval may comprise activity data from a most-recent sampling interval (e.g., about 5 minutes), and the third activity threshold may comprise about 50 steps. If fewer than 50 steps occur over the third evaluation interval, a fourth activity threshold comprising about 150 steps may be applied to activity data over a fourth evaluation interval comprising activity data aggregated from two most-recent sampling intervals (e.g., about 10 minutes). If fewer than 150 steps occur over the fourth evaluation interval, the system may determine that the activity state has ended. That is, only if neither the third nor the fourth activity thresholds are met does the system identify an end of the activity state. Based on this determination, the model 1620A may be configured to define an end time of the activity state as a boundary of the most-recent sampling interval. Put another way, the end time may be assigned to an earlier-in-time boundary or a later-in-time boundary of the most-recent sampling interval. This confirmation step may prevent premature termination of activity detection during brief pauses in exercise.

In some variations, gaps may occur in the activity data due to sensor communication interruptions, device removal, data compression, and/or other factors. To ensure accurate activity detection despite such gaps, the model 1620A may be configured to preprocess the activity data by inserting an inactivity value correlated with any gaps exceeding a threshold gap duration. Put differently, the model 1620A may transform the activity data to normalized activity data by correcting unrecorded or unreceived activity data. The threshold gap duration may be about 5 minutes to about 10 minutes, such as about 5 minutes, about 6 minutes, about 6.5 minutes, about 7 minutes, about 7.5 minutes, about 8 minutes, about 9 minutes, or about 10 minutes.

For example, if a gap in activity data exceeds the threshold gap duration (e.g., about 7.5 minutes), the model 1620A may insert one or more synthetic data points representing inactivity, such as a data point having zero steps. In some variations, the synthetic data point may be temporally positioned relative to the last valid data point by a predetermined interval, which may correspond to a sampling interval (e.g. about 5 minutes). This inserted inactivity value may be incorporated into the normalized activity data and may cause the model 1620A to properly segment activity states that span data gaps, ensuring that a single continuous activity state is not incorrectly inferred across a time period (e.g., a portion of a sampling interval, or one or more sampling intervals) when the user may have removed the device or experienced a sensor malfunction.

In some variations, the model 1620A may be implemented as a state machine that transitions between discrete states based on the activity threshold comparisons described above. The state machine may maintain a current activity state, which may be one of: inactive, active, or unknown. Transitions between states may occur based on evaluating activity data against the activity thresholds and evaluation intervals.

When the current state is inactive (or unknown), the state machine may check whether an activity onset should be identified by comparing activity data against the first activity threshold over the first evaluation interval. If the first threshold is satisfied, the state machine may evaluate the second evaluation interval against the second activity threshold to determine whether to transition to an active state with a start time of “now” or “prior”, as described above.

When the current state is active, the state machine may periodically check (e.g., every sampling interval) whether activity end should be identified by comparing current activity data against the additional activity threshold(s). If activity data falls below these activity thresholds, the state machine may perform a confirmation check over an extended evaluation interval before transitioning to an inactive state.

Moreover, in some variations, the model 1620A may classify activity data into intensity levels. Activity data received over a plurality of sampling intervals may be aggregated and classified as one or more of a low activity state, a moderate activity state, or a high activity state. The classification may be based on step counts, step cadence, and/or other metrics. For example, a low activity state may correspond to fewer than about 100 steps per evaluation interval (e.g., about 5 minutes), a moderate activity state may correspond to about 100 to about 200 steps per evaluation interval, and a high activity state may correspond to more than about 200 steps per evaluation interval. The activity intensity information may be provided to the user and may be displayed as a percentage of daily activity goals or as a breakdown of time spent at each intensity level.

In some variations, the model 1620A may additionally calculate daily activity statistics, such as the total number of active minutes, the total step count, and/or the percentage of the day spent at each activity intensity level. Activity data may be resampled at regular sample intervals (e.g., 5 minutes), the number of minutes at each activity intensity level may be calculated and grouped by day, and the results may be converted to percentages or displayed as progress toward a daily activity goal.

The output 1630A may comprise activity information. The activity information may comprise one or more data points, such as a start time of an activity state, an end time of an activity state, a duration of the activity state, and a total amount of activity (e.g., total steps) performed during the state. In some variations, the activity information may also include an intensity level classification. In variations where the activity end has not yet been determined (e.g., ongoing activity), the output 1630A may include the start time and current duration, but may omit or indicate as pending the end time. This activity information may be provided to the user via a graphical user interface (GUI) on the remote device, for example, as an activity interval on a health dashboard timeline. The GUI and presented output are described in further detail below with respect to the mobile application.

FIG. 16B depicts a flow diagram of a method 1650B for detecting activity of a user, which may be performed by the activity detection model described above. The method 1650B may be executed by a processor of a remote device, such as a mobile computing device, and may involve instructions stored on a non-transitory computer-readable medium that, when executed by one or more processors, cause the processors to perform the steps described herein. Generally, the remote device may receive data from one or monitoring devices, such as a first monitoring device (e.g., a wearable device with a sensor), which may be an activity monitoring device. In some variations, the method 1650B may be performed in real-time as activity data is received. In other variations, the method 1650B may be performed as post-processing of historical activity data. In some variations, as described with respect to FIGS. 13-15, in some variations, the method 1650B may be executed by or in conjunction with an analyte monitoring device.

The method 1650B may include receiving activity data from a monitoring device (e.g., an activity monitoring device comprising a wearable sensor) at step 1652B. The activity data may comprise a raw acceleration values (that may be processed to determine a step count) or a step count detected over a plurality of discrete sampling intervals. In some variations, each of the plurality of discrete sampling intervals may comprise any of the sampling intervals described herein, such as, for example, about 5 minutes. The activity data may be received wirelessly from the wearable sensor at periodic intervals, such as after each sampling interval, or may be received in a batch transfer comprising activity data from multiple sampling intervals. In some variations, the remote device may receive the activity data from the wearable sensor via a wireless communication protocol, such as a short-range wireless technology standard (e.g., Bluetooth or Bluetooth Low Energy).

The method 1650B may further include identifying a subset of the received activity data that meets a minimum activity threshold. In some variations, activity data from sampling intervals having fewer than a threshold number of steps (e.g., fewer than about 4 steps, fewer than about 10 steps, or fewer than about 50 steps) may be excluded from subsequent evaluation or may be treated as indicating an inactive state. The received activity data used for subsequent determining steps may comprise the subset of the received activity data that meets the minimum activity threshold. This filtering may reduce noise from incidental movements that do not represent meaningful physical activity.

In some variations, the method 1650B may include aggregating the activity data received over a plurality of discrete sampling intervals. For example, activity data from two or more (e.g., three, four, five, six, or more) consecutive sampling intervals may be summed or otherwise combined to produce an aggregated activity value for use in subsequent evaluation steps.

The method 1650B may further include determining an onset of an activity state by evaluating the received activity data at step 1654B. The determining may be based on (a) a first comparison of the received activity data to a first activity threshold over a first evaluation interval, and (b) a second comparison of the received activity data to a second, different activity threshold applied over a second, different evaluation interval. In some variations, the second evaluation interval may precede a portion of the first evaluation interval. For example, the second evaluation interval may comprise activity data from an earlier portion of the first evaluation interval, or may end immediately before the first evaluation interval begins.

In some variations, the second evaluation interval may be shorter than the first evaluation interval. For example, the first evaluation interval may be about 10 minutes and the second evaluation interval may be about 5 minutes. In some variations, the first activity threshold may be greater than the second activity threshold. For example, the first activity threshold may comprise about 300 steps over a 10-minute first evaluation interval (i.e., 30 steps/min), and the second activity threshold may comprise about 75 steps over a 5-minute second evaluation interval (i.e., 15 steps/min).

The first and second activity thresholds may be configured to reduce false positive activity detections. By requiring that activity data satisfy a higher threshold over a longer evaluation interval before evaluating a lower threshold over a shorter, earlier evaluation interval, the method may ensure that activity detection is triggered only when meaningful, sustained activity is occurring.

If the activity data in the first evaluation interval satisfies the first activity threshold and the activity data in the second evaluation interval satisfies the second activity threshold, the method may determine that the onset of the activity state occurred at or near the beginning of the second evaluation interval. If the activity data in the first evaluation interval satisfies the first activity threshold but the activity data in the second evaluation interval does not satisfy the second activity threshold, the method may determine that the onset of the activity state occurred at or near the current time. This dual-threshold approach may enable accurate determination of activity start times even when users gradually transition from inactivity to exercise.

The method 1650B may also include providing, via a display, activity information comprising a start time of the activity state based on the determined onset of the activity state at step 1656B. In some variations, providing the activity information may comprise storing the start time in a memory of the remote device. In some variations, providing the activity information may comprise transmitting the start time to a remote server, such as a cloud server. In some variations, providing the activity information may comprise displaying the start time on a graphical user interface (GUI) of the remote device.

In some variations, the activity information displayed may comprise the start time and a duration of the detected activity state. In some variations, providing the activity information may further comprise displaying a time-based visualization including one or more segments aligned along a common timeline, the one or more segments representing one or more activity states. The detected activity state may be displayed as a visually differentiated segment extending between the start time and an end time along the timeline. In some variations, the timeline may simultaneously display the activity information together with one or more of sleep information, medication information, or glucose information, thereby enabling a user to correlate activity patterns with physiological responses.

In some variations, the method 1650B may further include receiving user input to selectively display or hide one or more categories of information on the timeline. In some variations, the method 1650B may further include receiving user input selecting a temporal span of the timeline, such as a 12-hour view, a 24-hour view, or a multi-day view.

The method 1650B may also include additional steps not shown in FIG. 14B. For example, the method 1650B may include determining an end of the activity state based on a third, different activity threshold applied to the received activity data over a third, different evaluation interval. The third evaluation interval may be about 30 seconds to about 20 minutes, such as about 1 min to about 15 min, about 2 min to about 13 min, about 3 min to about 12 min, about 4 min to about 11 min, about 5 min to about 10 min, about 6 min to about 9 min, or about 7 min to about 8 min. In some variations, the third activity threshold may be lower than the first activity threshold. In some variations, the third activity threshold may be about 1 step to about 250 steps, such as about 5 steps to about 200 steps, about 10 steps to about 150 steps, about 25 steps to about 100 steps, about 30 steps to about 90 steps, about 40 steps to about 80 steps, about 45 steps to about 75 steps, about 50 steps to about 70 steps, or about 55 steps to about 65 steps. For example, the third activity threshold may comprise about 50 steps over a third evaluation interval comprising about 5 minutes.

In some variations, determining the end of the activity state may further comprise evaluating the received activity data over a fourth, different evaluation interval that is subsequent to the first, second, and third evaluation intervals. For example, if the activity data falls below the third activity threshold over the third evaluation interval, the method may further comprise evaluating whether the activity data falls below a fourth activity threshold over a fourth evaluation interval comprising a longer time period than the third evaluation interval, such as about 10 minutes. In some variations, the fourth activity threshold may comprise about 150 steps. Only if the activity data falls below both the third and fourth activity thresholds may the model determine that the activity state has ended. This confirmation step may prevent premature termination of activity detection during brief pauses in exercise.

In some variations, the activity information provided by the display may further comprise an activity end time based on the determined end of the activity state. In some variations, the activity information may comprise a duration of the activity state calculated from the start time and the end time.

In some variations, the method 1650B may further include, prior to determining the onset of the activity state, inserting an inactivity value correlated with any gaps in the activity data exceeding a threshold gap duration. The threshold gap duration may comprise about 5 minutes to about 10 minutes, such as about 7.5 minutes. For example, if a gap in the activity data exceeds the threshold gap duration, the method may insert a synthetic data point representing zero steps at a time point offset from the last valid data point by a predetermined interval, such as about 5 minutes. This preprocessing step may ensure that activity states are properly segmented when gaps occur in the activity data.

In some variations, the method 1650B may further include classifying the aggregated activity data as one or more of a low activity state, a moderate activity state, or a high activity state over a cumulative time duration comprising the plurality of discrete sampling intervals. For example, activity data corresponding to fewer than about 100 steps per evaluation interval may be classified as a low activity state, activity data corresponding to about 100 steps to about 200 steps per evaluation interval may be classified as a moderate activity state, and activity data corresponding to more than about 200 steps per evaluation interval may be classified as a high activity state. The activity intensity classification may be provided to the user as part of the activity information.

In some variations, the method 1650B may further include classifying the activity state as an activity type and displaying the activity type. The activity type may comprise one or more of walking, running, cycling, swimming, gardening, weightlifting, stretching, chores, or other user or application-defined activity categories. In some variations, the method may further include receiving user input modifying at least one of the start time, duration, or activity type associated with the activity state. For example, the GUI may present the detected activity as a selectable element, and upon user selection, may allow the user to adjust the start time, end time, duration, and/or activity type classification.

In some variations, the method 1650B may be performed by a remote device that receives the activity data from the monitoring device (e.g., from a wearable sensor thereof). The remote device may comprise a mobile computing device, such as a smartphone or tablet, communicably coupled with the wearable sensor. In some variations, the method 1650B may be repeated at regular intervals, such as after each sampling interval, to update the status of an ongoing activity state or to detect a new onset of an activity state.

### Inactivity Detection Model

The following description is directed to devices, systems, and methods for detecting inactivity states of a user by analyzing activity data and light data over one or more evaluation intervals using an inactivity detection model. Broadly, the inactivity detection model may be configured to differentiate between periods of inactivity (e.g., rest or sleep) and periods of wakefulness. This determination may be based on correlated reductions in movement and light exposure over time, optionally in combination with temporal features associated with the user's typical sleep schedule. The inactivity detection may be performed in real time or through post-processing of historical data. While the inactivity detection model may operate in conjunction with an analyte monitoring device—for example, to provide context for fluctuations in analyte (e.g., glucose) levels during sleep—the inactivity detection model and associated methods may also be implemented independently of any analyte monitoring hardware.

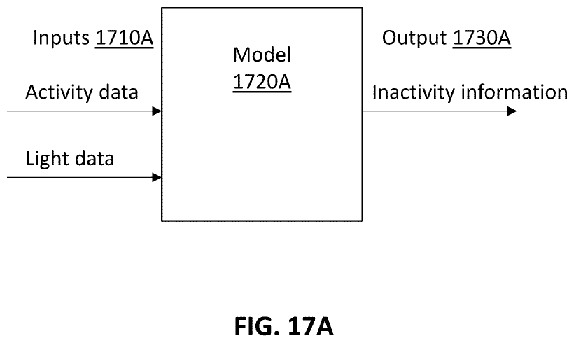

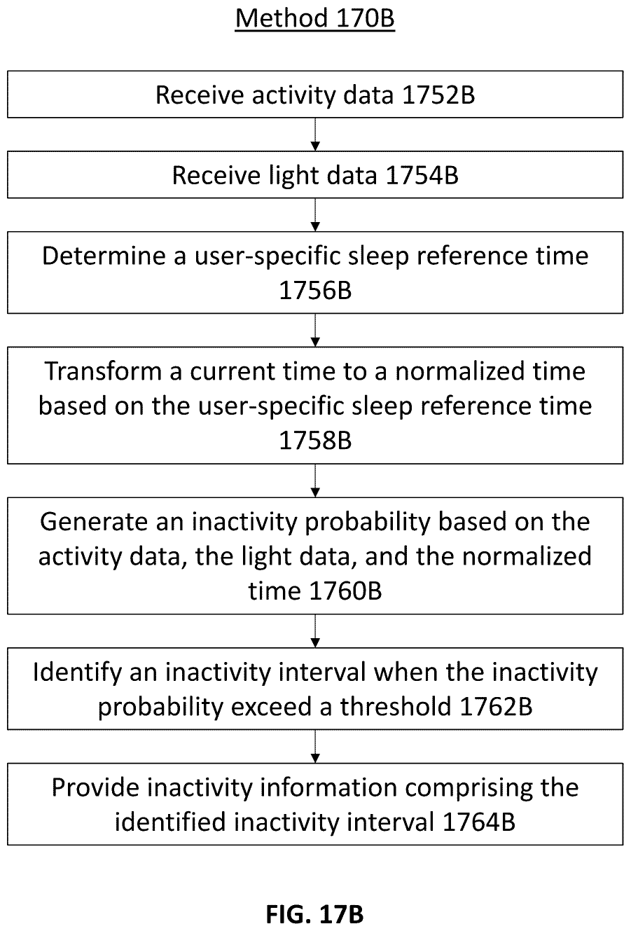

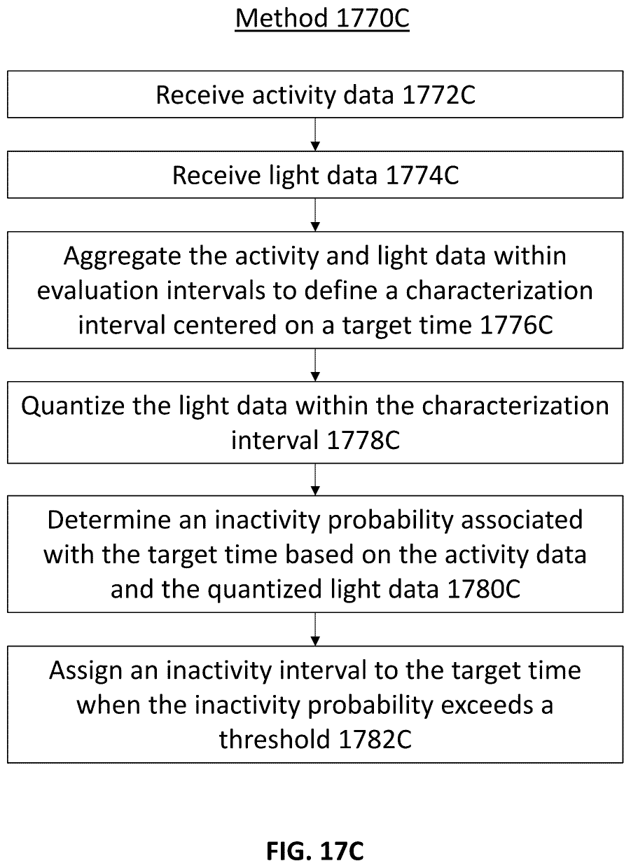

FIG. 17A depicts a block diagram of an inactivity detection model, which may be implemented by a remote device. The inactivity model may comprise a model 1720A, which may be a predictive model, algorithm, or state machine configured to receive one or more inputs 1710A and generate an output 1730A. In some variations, the model 1720A may be configured to generate an inactivity probability indicative of whether the user is in an inactivity state at a given time. The model 1720A may be executed by a processor within the remote device, which may store the model in a non-transitory computer-readable medium. The remote device may be communicably coupled with at least a first wearable device that transmits one or more of the inputs 1710A thereto.

In some variations, the model 1720A may comprise a neural network. For example, the neural network may comprise one or more hidden layers, such as a first hidden layer and a second hidden layer. In some variations, the neural network may comprise about 50 to about 500 neurons in a first hidden layer and about 25 to about 300 neurons in a second hidden layer, such as about 200 neurons and about 100 neurons, respectively. In some variations, the neural network may use an activation function, such as a ReLU activation function. In other variations, the model 1720A may comprise a machine learning model, a random forest model, a support vector machine, or another classification model.

In some variations, the model 1720A may be trained using reference data obtained from a plurality of users. The reference data may comprise historical activity data and light data correlated with user-verified sleep logs or data from reference devices such as fitness trackers. The plurality of users may comprise at least 10 users, at least 20 users, at least 30 users, at least 50 users, at least 75 users, at least 100 users, at least 250 users, at least 500 users, at least 750 users, or at least 1,000 users. In some variations, the reference data may be divided into training and validation sets, such as data from about 20 to about 30 sensors for training and data from about 5 to about 15 sensors for validation. In some variations, the reference data may require manual cleaning or reconciliation, such as reconciling inconsistencies between user sleep logs and fitness tracker data.

The inputs 1710A to the model 1720A may comprise activity data and light data. The activity data may include any data indicative of physical movement or lack thereof by the user. For example, the activity data may comprise a step count. The activity data may be received from a first wearable device, such as a wearable sensor, a smartwatch, a smartphone, a pedometer, or an analyte monitoring device. In some variations, the activity data may be generated by a kinetic sensor, such as an accelerometer, which may output processed step count data rather than raw acceleration values.

The light data may comprise measurements from a light sensor. In some variations, the light data may be received from the first wearable device. In other variations, the light data may be received from a second wearable device different from the first wearable device. The light data may be indicative of an ambient light environment experienced by the user, which may correlate with whether the user is in a dark environment consistent with rest or sleep.

In some variations, the activity data and the light data may be received in discrete sampling intervals. Each sampling interval may comprise about 30 seconds to about 60 minutes, such as about 1 minute to about 30 minutes, about 2 minutes to about 30 minutes, about 3 minutes to about 25 minutes, about 4 minutes to about 20 minutes, about 5 minutes to about 15 minutes, about 6 minutes to about 12 minutes, about 7 minutes to about 10 minutes, about 7.5 minutes to about 9 minutes, or about 8 minutes to about 8.5 minutes. In some variations, each sampling interval may comprise about 2 minutes to about 30 minutes, such as about 5 minutes, about 7.5 minutes, about 10 minutes, about 15 minutes, about 20 minutes, about 25 minutes, or about 30 minutes. In some variations, the sampling interval may be about 30 seconds, about 1 minute, about 2 minutes, about 3 minutes, about 4 minutes, about 5 minutes, about 6 minutes, about 7 minutes, about 7.5 minutes, about 8 minutes, about 9 minutes, about 10 minutes, about 12 minutes, about 15 minutes, about 20 minutes, about 25 minutes, about 30 minutes, about 45 minutes, about 60 minutes, or more than about 60 minutes. In some variations, the sampling interval may be less than about 5 minutes, about 5 minutes, or more than about 5 minutes.

In some variations, the model 1720A may evaluate data over one or more evaluation intervals. An evaluation interval may define a time period over which activity data and light data are aggregated and evaluated to classify a target time. In some variations, an evaluation interval may span one or more sampling intervals. In some variations, an evaluation interval may comprise about 1 minute to about 12 hours, such as about 5 minutes to about 8 hours, about 10 minutes to about 6 hours, about 15 minutes to about 4 hours, about 30 minutes to about 3 hours, about 45 minutes to about 2.5 hours, or about 1 hour to about 2 hours. In some variations, an evaluation interval may comprise about 10 minutes to about 4 hours, such as about 15 minutes to about 3 hours, about 25 minutes to about 2.5 hours, about 30 minutes to about 2.25 hours, about 40 minutes to about 2 hours, about 50 minutes to about 1.75 hours, or about 1 hour to about 1.5 hours. In some variations, an evaluation interval may comprise about 5 minutes, about 10 minutes, about 15 minutes, about 20 minutes, about 30 minutes, about 45 minutes, about 1 hour, about 1.25 hours, about 1.5 hours, about 2 hours, about 2.5 hours, about 3 hours, about 4 hours, about 6 hours, about 8 hours, or about 12 hours.

In some variations, the activity data may be standardized or normalized prior to input into the predictive model. For example, step counts may be normalized to have a mean value of about 4 to about 7 and a standard deviation of about 15 to about 25, such as a mean of about 5 to about 6 and a standard deviation of about 19 to about 20.

In some variations, the model 1720A may be configured to discretize the light data into light level values. Discretizing the light data may comprise mapping a raw light measurement to one of a plurality of discrete light level values. In some variations, the plurality of discrete light level values may comprise 2 to 10 levels, such as 3 to 8 levels, 4 to 7 levels, or 5 levels. In some variations, the light level values may range from 1 to 5. In some variations, the light level values may be about 1, about 2, about 3, about 4, or about 5.

In some variations, the discretized light level values may range from 1 to 5, where lower values (e.g., levels 1-2) indicate darker environments and higher values (e.g., levels 4-5) indicate brighter environments. For example, a light level value of 1 may correspond to a dark environment consistent with nighttime sleep, while a light level value of 5 may correspond to a bright environment consistent with daytime wakefulness. In some variations, a monitoring device may output discretized light level values directly.

In other variations, the remote device may convert raw lux or millilux measurements into the discretized light level values. In such variations, the raw light measurements may be expressed as about 0.001 lux to about 100,000 lux, such as about 0.01 lux to about 10,000 lux, about 0.1 lux to about 1,000 lux, about 1 lux to about 500 lux, about 5 lux to about 200 lux, about 10 lux to about 100 lux, or about 20 lux to about 50 lux. In some variations, the raw light measurement may be about 0.001 lux, about 0.01 lux, about 0.1 lux, about 0.5 lux, about 1 lux, about 2 lux, about 5 lux, about 10 lux, about 20 lux, about 50 lux, about 100 lux, about 200 lux, about 500 lux, about 1,000 lux, about 5,000 lux, about 10,000 lux, or more than about 10,000 lux.

In some variations, the input 1710A may further comprise temporal features. For example, the model 15020A may receive as an input a current time or a time associated with a data point being evaluated. In some variations, the model 1720A may receive as an input a user-specific sleep reference time. The user-specific sleep reference time may comprise a typical sleep time or a typical wake time for the user. In some variations, determining the user-specific sleep reference time may comprise receiving user input indicative of a typical sleep time or typical wake time. For example, the user may be prompted to enter their normal bedtime and normal wake time via a graphical user interface (GUI) on the remote device, as described in detail below.

In some variations, the model 1720A may transform a current time to a normalized time based on the user-specific sleep reference time. Transforming the current time may comprise shifting the current time relative to the user-specific sleep reference time to constrain the normalized time within a bounded range. In some variations, the bounded range may comprise about −12 hours to about +12 hours relative to the user-specific sleep reference time. This transformation may address a technical challenge arising from the cyclical nature of time: without transformation, a discontinuity exists between hour 23 and hour 0 (midnight), which may cause errors for models that rely on continuous input features. By shifting the time representation relative to the user's expected wake time, the discontinuity may be moved to a time when the user is least likely to be transitioning between sleep and wakefulness, thereby improving model performance.

For example, the normalized time may be calculated by subtracting the user-specific sleep reference time (e.g., the normal wake time) from a current time. If the resulting value exceeds a first threshold (e.g., +12 hours), a full day value (e.g., 24 hours) may be subtracted to shift the normalized time into the bounded range. This transformation may produce a normalized time value where negative values correspond to times before the expected wake time (i.e., times when the user is more likely to be asleep) and positive values correspond to times after the expected wake time (i.e., times when the user is more likely to be awake). The gradient of the normalized time feature may be largest near zero (i.e., near the expected wake time), which may help the model 1720A distinguish between sleep and wake states during the transition period.

In some variations, the inputs 1710A may further comprise a proximity to the user-specific sleep reference time. The proximity may comprise a distance in time to an expected sleep time, a distance in time to an expected wake time, or both. For example, the predictive model 1720A may receive as inputs both a “proximity to expected sleep time” feature and a “proximity to expected wake time” feature. These proximity features may enable the model 1720A to account for the user's typical sleep schedule when determining an inactivity probability.

The model 1720A may be configured to classify whether a user is in an inactivity state (e.g., asleep) at a given target time. To do so, the model 1720A may analyze activity data and light data from a characterization interval surrounding the target time, and may generate an inactivity probability representing the likelihood that the user is asleep at that target time. If the inactivity probability exceeds an inactivity threshold for at least a minimum evaluation interval (e.g., at least about one hour), the model 1720A may identify an inactivity interval and assign a sleep state to that time period. This multi-step approach-generating a probability, then applying an inactivity threshold over a minimum evaluation interval-may reduce false positive detections that might otherwise occur when the user is sedentary but awake, such as when sitting at a desk or watching television in a dark room.

To generate the inactivity probability, the model 1720A may evaluate activity data and light data from evaluation intervals both before and after the target time. In some variations, the model 1720A may aggregate the activity data and the light data within a first evaluation interval preceding the target time and a second evaluation interval following the target time to define a characterization interval centered on the target time. In some variations, each of the first evaluation interval and the second evaluation interval may comprise about 10 minutes to about 4 hours, such as about 15 min to about 3 hr, about 25 min to about 2.5 hr, about 30 min to about 2.25 hr, about 40 min to about 2 hr, about 50 min to about 1.75 hr, or about 1 hr to about 1.5 hr. For example, one or both of the first or second evaluation intervals may be about 10 minutes, about 15 minutes, about 30 minutes, about 45 minutes, about 1 hour, about 1.25 hr, or about 1.5 hours. If each of the first and second evaluation intervals comprises about one hour, the characterization interval may span about two hours centered on the target time (i.e., about one hour before and about one hour after the target time).

In some variations, the characterization interval may span a duration of about 20 minutes to about 12 hours centered on the target time, such as about 30 minutes to about 8 hours, about 45 minutes to about 6 hours, about 1 hour to about 4 hours, about 1.5 hours to about 3.5 hours, or about 2 hours to about 3 hours. In some variations, the characterization interval may span about 1 hour, about 1.5 hours, about 2 hours, about 2.5 hours, about 3 hours, about 4 hours, about 5 hours, about 6 hours, about 8 hours, about 10 hours, or about 12 hours.

In some variations, the first evaluation interval preceding the target time and the second evaluation interval following the target time may have equal durations. In other variations, the first and second evaluation intervals may have different durations. In some variations, the first evaluation interval may be longer than the second evaluation interval. In other variations, the second evaluation interval may be longer than the first evaluation interval. In some variations, the first and second evaluation intervals may each be about 10 minutes to about 4 hours, such as about 15 minutes to about 3 hours, about 25 minutes to about 2.5 hours, about 30 minutes to about 2.25 hours, about 40 minutes to about 2 hours, about 50 minutes to about 1.75 hours, or about 1 hour to about 1.5 hours. In some variations, one or both of the first and second evaluation intervals may be about 10 minutes, about 15 minutes, about 20 minutes, about 25 minutes, about 30 minutes, about 45 minutes, about 1 hour, about 1.25 hours, about 1.5 hours, about 2 hours, about 3 hours, or about 4 hours.

This bidirectional evaluation approach may introduce a lag in inactivity detection, as the model 1720A may wait until data from the subsequent evaluation interval is available before classifying a given time point. For example, if each evaluation interval comprises about one hour, the system may not classify whether the user was asleep at a given time until about one hour after that time. However, this approach may improve accuracy by providing the model 1720A with a more complete picture of the user's activity and light patterns surrounding the target time. In some variations, the model 1720A may be non-recursive. That is, the model 1720A may not require past outputs (e.g., prior inactivity classifications) as inputs to generate a given output. Instead, the model 1720A may use any segment of data spanning the characterization interval (e.g., about 2 hours) to make a prediction, without reference to prior predictions.

The model 1720A may generate the inactivity probability based on the activity data, the light data, and the normalized time (and/or the proximity to the user-specific sleep reference time). The inactivity probability may comprise a value between 0 and 1 that represents a likelihood that the user is in an inactivity state (e.g., asleep) at the target time. In some variations, determining the inactivity probability may comprise correlating a reduction in activity data (e.g., step count) with a reduction in light data (e.g., quantized light level values) within the characterization interval. In some variations, the model 1720A may treat low activity as activity data below an activity threshold. When the activity data comprises step count, the activity threshold may comprise about 0 steps to about 50 steps per evaluation interval, such as about 0 to about 25 steps, about 1 to about 20 steps, about 2 to about 15 steps, about 3 to about 10 steps, or about 4 to about 6 steps per evaluation interval. In some variations, the activity threshold may be about 0 steps, about 1 step, about 2 steps, about 3 steps, about 4 steps, about 5 steps, about 6 steps, about 8 steps, about 10 steps, about 15 steps, about 20 steps, about 25 steps, about 30 steps, about 40 steps, or about 50 steps per evaluation interval.

For example, if the activity data is below an activity threshold (e.g., fewer than about 1, 2, 3, 4, or 5 steps) and the light data indicates low light level values (e.g., level 1 or level 2) during the characterization interval, the model 1720A may output a higher inactivity probability that is closer to 1 than to 0.

Once the inactivity probability is generated for a given target time, the model 1720A may determine whether to identify an inactivity interval based on whether the inactivity probability exceeds an inactivity threshold for at least a minimum evaluation interval. In some variations, the inactivity threshold may be a value between 0 and 1, such as about 0.1 to about 0.99, about 0.2 to about 0.95, about 0.3 to about 0.9, about 0.4 to about 0.9, about 0.45 to about 0.85, about 0.5 to about 0.8, about 0.55 to about 0.75, or about 0.6 to about 0.7. In some variations, the inactivity threshold may be about 0.4, about 0.45, about 0.5, about 0.55, about 0.6, about 0.65, about 0.7, about 0.75, about 0.8, about 0.85, about 0.9, or about 0.95.

In some variations, the minimum evaluation interval may comprise about 10 minutes to about 6 hours, such as about 15 minutes to about 4 hours, about 20 minutes to about 3 hours, about 30 minutes to about 2.5 hours, about 45 minutes to about 2 hours, or about 60 minutes to about 1.5 hours. In some variations, the minimum evaluation interval may comprise about 30 minutes, about 45 minutes, about 60 minutes, about 75 minutes, about 90 minutes, about 2 hours, about 3 hours, about 4 hours, about 5 hours, or about 6 hours. In some variations, the minimum evaluation interval may comprise at least about 60 minutes.

In some variations, the minimum evaluation interval may comprise a plurality of consecutive evaluation intervals. For example, when each evaluation interval corresponds to about 2 minutes, about 5 minutes, about 7.5 minutes, about 10 minutes, or about 15 minutes, the minimum evaluation interval may comprise about 4 to about 120 consecutive evaluation intervals, such as about 6 to about 60 consecutive evaluation intervals, about 8 to about 48 consecutive evaluation intervals, about 10 to about 36 consecutive evaluation intervals, or about 12 to about 24 consecutive evaluation intervals. In one example, when each evaluation interval corresponds to about 5 minutes, the minimum evaluation interval may comprise about 6, about 8, about 10, about 12, about 14, about 16, about 18, about 20, or about 24 consecutive evaluation intervals, such as about 12 consecutive evaluation intervals totaling about 60 minutes.

This minimum evaluation interval requirement may address a technical challenge in distinguishing true sleep from sedentary wakefulness. Because sleep and sedentary behavior may produce similar patterns of low activity and low ambient light, relying solely on instantaneous probability values may result in false positive sleep detections. By requiring the inactivity probability to exceed the inactivity threshold for at least about, e.g., one hour, the model 1720A may reliably distinguish between true sleep states and mere periods of sedentary wakefulness. For example, if a user is sitting still in a dark room for 30 minutes but then begins moving, the inactivity probability may not persist above the inactivity threshold for the full minimum evaluation interval, and the system may correctly classify the user as awake rather than asleep.

In some variations, an inactivity state of a user may be a sleep state. In some variations, an awake state may be assigned when the inactivity probability exceeds the inactivity threshold but does not persist for the minimum evaluation interval.

The output 1730A may comprise inactivity information. The inactivity information may comprise one or more data points, such as a start time of an inactivity interval, an end time of an inactivity interval, and/or a duration of the inactivity interval. In some variations, the inactivity information may comprise an identified inactivity interval, which may define a time range during which the user was determined to be in an inactivity state (e.g., asleep). In some variations, the model 1720A may be configured to assign an inactivity interval to a target time when the inactivity probability exceeds the inactivity threshold for at least the minimum evaluation interval.

This inactivity information may be provided to the user via a graphical user interface (GUI) on the remote device. In some variations, the inactivity information may be displayed as an inactivity interval on a dashboard timeline. The GUI and presented output are described in further detail below with respect to the mobile application.

FIG. 17B depicts a flow diagram of a method 1750B for detecting inactivity of a user, which may be performed by the inactivity detection model described above. The method 1750B may be executed by a processor of a remote device, such as a mobile computing device, and may involve instructions stored on a non-transitory computer-readable medium that, when executed by one or more processors, cause the processors to perform the steps described herein. In some variations, the method 1750B may be performed in real-time as activity data and light data are received. In other variations, the method 1750B may be performed as post-processing of historical data. The method 1750B may be performed in conjunction with the method 1770C described below, and/or in conjunction with the activity detection methods described above (e.g., method 1650B). For example, the remote device may perform both activity detection and inactivity detection to provide a comprehensive view of the user's daily activity and rest patterns. Generally, the remote device may receive data from one or monitoring deivecs, such as a first monitoring device (e.g., a wearable device with a sensor), and a second, different monitoring device (e.g., a wearable device with a different sensor). Alternatively, the remote device may receive data from a single monitoring device (e.g., a wearable device with a plurality of sensor types). In some variations, as described with respect to FIGS. 13-15, in some variations, the method 1770B may be executed by or in conjunction with an analyte monitoring device.

The method 1750B may include receiving activity data from a first wearable device at step 1752B. The activity data may comprise a step count detected over a plurality of discrete sampling intervals. In some variations, the activity data and the light data may be received in discrete sampling intervals of about 5 minutes. The activity data may be received wirelessly from the first wearable device at periodic intervals, such as after each sampling interval, or may be received in a batch transfer comprising data from multiple sampling intervals.

The method 1750B may further include receiving light data from the first monitoring (e.g., wearable) device or a second, different monitoring (e.g., wearable) device at step 1754B. The light data may comprise measurements from an ambient light sensor. In some variations, the light data may be received from the same wearable device that provides the activity data (i.e., the first and second monitoring devices may be the same device). In other variations, the light data may be received from a second monitoring device that is different from the first monitoring device. In some variations, the method 1750B may further include discretizing the light data into light level values. In some variations, the light level values may comprise values of 1 to 5, where lower values indicate darker environments and higher values indicate brighter environments.

The method 1750B may further include determining a user-specific sleep reference time at step 1756B. The user-specific sleep reference time may comprise a typical sleep time or a typical wake time for the user. The typical sleep time may be a time or range of times at which the user typically goes to sleep. The typical wake time may be a time or range of times at which the user typically wakes up. In some variations, determining the user-specific sleep reference time may comprise receiving user input indicative of a typical sleep time or typical wake time. For example, the user may be prompted to enter their normal bedtime and normal wake time via a graphical user interface on the remote device. In some variations, the user-specific sleep reference time may be based on population (e.g., cohort or general population) means or default values if user input is not provided. For example, a default sleep time of about 11:00 PM and a default wake time of about 7:00 AM may be used. In some variations, the typical sleep and/or typical wake times may be adjusted by the user. Additionally, in some variations, the typical sleep and/or typical wake times may be different for different days of the week.

The method 1750B may further include transforming a current time to a normalized time based on the user-specific sleep reference time at step 1758B. In some variations, transforming the current time may comprise shifting the current time relative to the user-specific sleep reference time to constrain the normalized time within a bounded range. In some variations, the bounded range may comprise about −12 hours to about +12 hours relative to the user-specific sleep reference time. This transformation may address a discontinuity that would otherwise exist between hour 23 and hour 0 (midnight) in a 24-hour time representation. By shifting the time representation relative to the user's expected wake time, the discontinuity may be moved to a time when the user is least likely to be transitioning between sleep and wakefulness.

For example, the normalized time may be calculated by subtracting the user-specific sleep reference time (e.g., the normal wake time) from the current time. If the resulting value exceeds about +12 hours, a full day value (e.g., 24 hours) may be subtracted to shift the normalized time into the bounded range. Conversely, if the resulting value is less than about −12 hours, a full day value may be added. This transformation may produce a normalized time value where negative values correspond to times before the expected wake time (i.e., times when the user may be more likely to be asleep) and positive values correspond to times after the expected wake time (i.e., times when the user may be more likely to be awake).

The method 1750B may further include generating an inactivity probability based on the activity data, the light data, and the normalized time at step 1760B. The inactivity probability may comprise a value between 0 and 1 that represents a likelihood that the user is in an inactivity state (e.g., asleep) at a target time. In some variations, a model (e.g., a predictive model) may receive the activity data, the light data, and the normalized time as input to generate the inactivity probability. In some variations, the model may further receive as an input a proximity to the user-specific sleep reference time, such as a proximity in time to an expected sleep time and/or a proximity in time to an expected wake time. In some variations, the predictive model may comprise a layered neural network, such as a neural network with one or more hidden layers.

In some variations, the inactivity probability may be generated using the activity data and the light data from time intervals before and after the current time. For example, the system (e.g., via the model) may evaluate activity data and light data from a characterization interval spanning about one hour before and about one hour after the time point being classified. This bidirectional evaluation may improve accuracy by providing the predictive model with a more complete picture of the user's activity and light patterns surrounding the target time.

The method 1750B may further include identifying an inactivity interval when the inactivity probability exceeds a threshold for at least a minimum evaluation interval at step 1762B. In some variations, the minimum evaluation interval may comprise a plurality of consecutive evaluation intervals, and the inactivity interval may be identified when the inactivity probability exceeds the threshold over the plurality of consecutive evaluation intervals. In some variations, each of the plurality of consecutive evaluation intervals may correspond to at least about one hour. For example, the minimum evaluation interval may require the inactivity probability to exceed the inactivity threshold for at least about 25 minutes, at least about 30 minutes, at least about 35 minutes, at least about 40 minutes, at least about 45 minutes, at least about 50 minutes, at least about 55 minutes, at least about 60 minutes, at least about 65 minutes, at least about 70 minutes, at least about 75 minutes, or at least about 90 minutes. This minimum duration requirement may reduce false positive inactivity detections that might otherwise occur when the user is sedentary but awake.

The method 1750B may further include providing, via a display, inactivity information comprising the identified inactivity interval at step 1764B. The inactivity information may comprise a start time, a duration, and/or an end time for the inactivity interval. In some variations, providing the inactivity information may comprise storing the inactivity interval in a memory of the remote device. In some variations, providing the inactivity information may comprise transmitting the inactivity interval to a remote device, such as a cloud server. In some variations, providing the inactivity information may comprise displaying the inactivity interval on a graphical user interface (GUI) of the remote device, such as on a health dashboard timeline. The inactivity interval may be displayed in temporal alignment with other physiological or contextual information, such as activity intervals, medication events, or analyte (e.g., glucose) readings.

FIG. 17C depicts a flow diagram of a method 1770C for determining an inactivity of a user, which may be performed by the inactivity detection model described above. The method 1770C may represent another aspect of inactivity detection that may be used in conjunction with the method 1750B described above, and/or in conjunction with the activity detection methods described above (e.g., method 1650B). The method 1770C may be executed by a processor of a remote device, such as a mobile computing device, and may involve instructions stored on a non-transitory computer-readable medium that, when executed by one or more processors, cause the processors to perform the steps described herein. Generally, the remote device may receive data from one or monitoring devices, such as a first monitoring device (e.g., a wearable device with a sensor), and a second, different monitoring device (e.g., a wearable device with a different sensor). Alternatively, the remote device may receive data from a single monitoring device (e.g., a wearable device with a plurality of sensor types). In some variations, as described with respect to FIGS. 13-15, in some variations, the method 1770C may be executed by or in conjunction with an analyte monitoring device.

The method 1770C may include receiving activity data from a first monitoring (e.g., wearable) device at step 1772C. The activity data may comprise a step count detected over a plurality of discrete sampling intervals. In some variations, each discrete sampling interval may comprise about 5 minutes.

The method 1770C may further include receiving light data from the first monitoring (e.g., wearable) device or a second, different monitoring (e.g., wearable device) at step 1774C. The light data may comprise measurements from an ambient light sensor. In some variations, the light data may be received from the same device that provides the activity data. In other variations, the light data may be received from a different device.

The method 1770C may further include aggregating the activity data and the light data within evaluation intervals to define a characterization interval centered on a target time at step 1776C. In some variations, the aggregating may comprise aggregating the activity data and the light data within a first evaluation interval preceding the target time and a second evaluation interval following the target time. The characterization interval may thus span both preceding and following time periods relative to the target time. In some variations, each of the first evaluation interval and the second evaluation interval may comprise about one hour. Thus, the characterization interval may span about two hours centered on the target time (i.e., about one hour before and about one hour after the target time). This bidirectional aggregation may enable the method to evaluate the user's activity and light patterns both leading up to and following the target time, which may improve the accuracy of inactivity classification.

The method 1770C may further include quantizing the light data within the characterization interval at step 1778C. Quantizing the light data may comprise assigning light level values to measurements within the light data. In some variations, the light level values may comprise values of 1 to 5, where lower values indicate darker environments and higher values indicate brighter environments. In some variations, the light data may already be quantized by the wearable device prior to transmission to the remote device. In other variations, the remote device may quantize raw light measurements (e.g., lux or millilux values) into the discrete light level values.

The method 1770C may further include determining, based on the activity data and the quantized light data within the characterization interval, an inactivity probability associated with the target time at step 1780C. The inactivity probability may comprise a value between 0 and 1 that represents a likelihood that the user is in an inactivity state (e.g., asleep) at the target time. In some variations, determining the inactivity probability may comprise correlating a reduction in the step count with a reduction in light level values within the characterization interval. For example, if the activity data indicates low or zero step counts and the quantized light data indicates low light level values (e.g., level 1 or level 2) during the characterization interval, the method may determine a higher inactivity probability.

The method 1770C may further include assigning an inactivity interval to the target time when the inactivity probability exceeds a threshold for at least a minimum evaluation interval at step 1782C. In some variations, the minimum evaluation interval may comprise a continuous duration of about 60 minutes. In some variations, an inactivity state may comprise a sleep state. In some variations, an awake state may be assigned when the inactivity probability exceeds the threshold but does not persist for the minimum evaluation interval. For example, if the user is sitting still in a dark room but the inactivity probability does not exceed the threshold for at least about one hour, the method may assign an awake state rather than a sleep state.

The method 1770C may also include additional steps not shown in FIG. 15C. For example, the method 1770C may include providing, via a display, inactivity information comprising the assigned inactivity interval. The inactivity information may be displayed on a graphical user interface (GUI) of the remote device, such as on a health dashboard timeline that simultaneously displays inactivity intervals together with activity intervals, medication events, or glucose readings.

### Mobile Application

Further described herein are user interfaces that may incorporate the display of inactivity information (e.g., rest data or sleep data), activity information, baseline information, analyte data, and/or medication data. The user interfaces may be generated by a mobile application executed on a mobile computing device. As used herein, the mobile computing device may be a remote device as described elsewhere herein. The mobile application and the user interfaces generated thereby may be collectively referred to herein as a graphical user interface (GUI).

In some variations, the mobile application may operate in conjunction with one or more monitoring devices (e.g., a wearable analyte monitoring device) as described elsewhere herein. For example, the mobile computing device may receive analyte data, activity data, and/or light data from the wearable analyte monitoring device via wireless communication (e.g., NFC, Bluetooth, or Bluetooth Low Energy). In some variations, the mobile application may operate in conjunction with other monitoring devices, such as a fitness tracker, a smartwatch, a pedometer, or other wearable sensors. In some variations, the mobile application may receive data from a plurality of monitoring devices. In some variations, the mobile application may perform activity detection and/or inactivity detection as described herein without requiring an analyte monitoring device. For example, the mobile application may receive activity data and/or light data from a wearable sensor that does not include analyte sensing capabilities, and may perform the activity detection and inactivity detection methods described herein based on that data. Thus, while the following description may reference an analyte monitoring device as an exemplary data source, the systems and methods described herein are not limited to use with an analyte monitoring device and may be practiced with any combination of wearable and remote devices.

Referring again to FIG. 1, the mobile computing device 102 may comprise at least one processor and at least one memory storing instructions that, when executed by the processor, cause the mobile computing device to execute and perform the activity and inactivity detection models/methods described herein. The mobile computing device may further comprise a display and physical interface components (e.g., touch-sensitive screens, buttons, or haptic engines) to enable the generation and operation of the GUI. The methods and algorithms described herein may be implemented as instructions stored on a non-transitory computer-readable medium. Such a non-transitory computer-readable medium may be located on the mobile computing device, a cloud-based server, or a combination thereof.

The mobile application executed by the mobile computing device 102 may generate a series of user interfaces that provide the user with data (e.g., the analyte data, the rest data, and/or the activity data), insights, and/or resources. The data, insights, and/or resources may include or reference one or more links, the selection of which may provide additional information or may direct the user to one or more other sources of information, such as to a website through an internet browser.

In some variations, the mobile application executed by the mobile computing device 102 accesses a catalog of insights related to educational information and the analyte data, rest data, and activity data. Each insight may be associated with one or more characteristics, such as, for example, characteristics of the analyte monitoring device 110, the analyte data, rest data, activity data, and/or other data available to the mobile computing device 102. In some variations, an insight may be associated with baseline data on a user that is neither exercising (active) nor intentionally resting or sleeping (inactive). In some the user interfaces described herein, one or more insights may be rendered so that the user is provided with an opportunity to better understand their analyte level and impacts to their analyte level.

In some variations, an insight engine is implemented to select an insight (a message) to be rendered on a user interface. The insight engine may utilize pattern matching, which includes receiving input data including, for example, status of the analyte monitoring device 110, analyte data, activity data, ambient light sensor data, sleep data, meal data, analyte spikes, time information, data related to the user's routine, edited or added sleep, edited or added activity, time-in-range goals, and/or activity goals. A pattern matching module takes the input data and generates a set of events, which represents a current state of the user. An insights file includes a plurality of messages, each of which has a set of triggering events (an inclusion event list) and a set of precluding events (an exclusion event list). The set of events generated by the pattern matching module is filtered, or matched, against the insights file, with the triggering events and the precluding events, to narrow the insights to a set of messages. Each message is associated with a tier and a weight. Tiers may be used whenever the user or the analyte monitoring device 110 include an event or state that requires a specific message to be shown. Weights may be used under many conditions. A message with a higher weight is more likely to be selected. A message is selected from the set of messages based on tiers and weights. In some variations, messages may expire after a set time or based on user interaction with the mobile application.

In some variations, the mobile application executed by the mobile computing device 102 generates a series of user interfaces that allow the user to enter user information that the mobile computing device 102 uses for displaying and processing of data. The user-provided information may include user demographics, user registration information, user medication information, user sleep information, and user-defined goals.

One or more user interface controls may be provided on the user interfaces and may be configured to display data, manipulate data, and hide data based upon user selection. The user interfaces generated by the mobile application may, in some variations, transition to other user interfaces based on user selection of the one more user interface controls, which may include, for example, icons, buttons, graphics, drop-down filters, scrollable icons, or other selectable options. For example, the user interfaces may include drop-down windows or scrollable icons, selection of which results in one or more of providing an alternate user interface, populating additional data or details on the user interface, and hiding data on the user interface.

In some variations, the mobile application generates an analyte results user interface that provides a view of the user's analyte level at a given time. The analyte results user interface may be updated and displayed on the mobile application in response to the user scanning the analyte monitoring device 110 with the mobile computing device 102. The data shown may be that from the most recent time that the user has scanned the analyte monitoring device 110 or from the most recent time that the mobile computing device 102 receives analyte data from the analyte monitoring device 110.

The analyte results user interface may include a span of analyte values and may be arranged in sub-spans (e.g., bins or buckets) of values. Each sub-span may represent a range of potential values of the analyte, and the sub-span representative of the range in which the user's analyte level is at a given time may be marked or emphasized to convey the range to the user. For example, the sub-span may be highlighted or illuminated, or an icon may be positioned in the sub-span to show in which range of values the user's analyte level is at a given time. The analyte results user interface thus provides a snapshot view, informing the user of their current analyte level within a range of values.

In some variations, analyte values may be defined as in-range and out-of-range. In-range values may be those considered to be healthy and/or desirable. In some variations, the in-range values are industry-standard or industry-defined values. In some variations, the in-range values are defined by a medical provider or input or defined by the user. Out-of-range values are those considered to be unhealthy and/or undesirable.

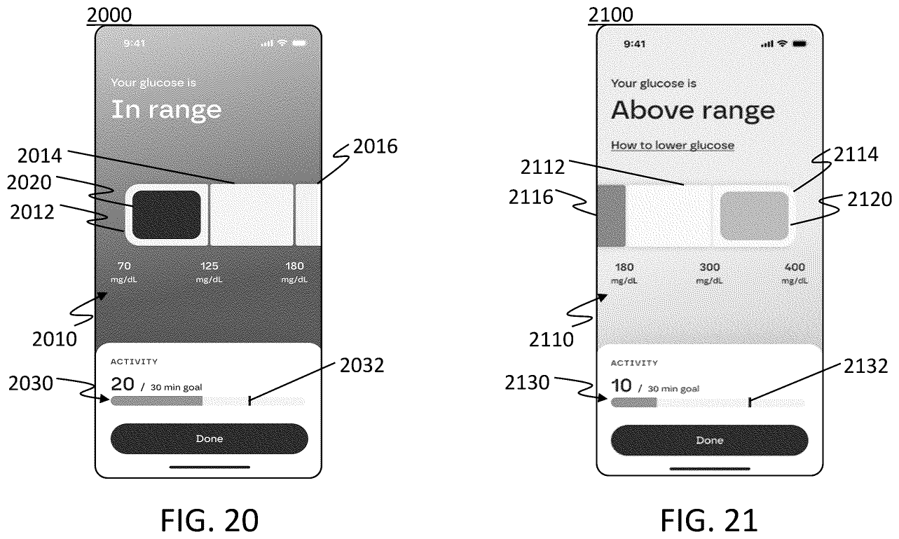

FIG. 20 is an exemplary depiction of an in-range analyte results user interface 2000 generated by the mobile application according to a variation. The in-range analyte results user interface 2000 includes a span (or range) 2010 of analyte values divided between three sub-spans (or buckets) 2012, 2014, and 2016, each of which represents a range of analyte monitoring data. A first sub-span 2012 represents a first in-range group of values, and a second sub-span 2014 represents a second in-range group of values. A third sub-span 2016 is also shown and conveys to the user that moving beyond the second sub-span moves the user into an out-of-range value.

FIG. 21 is an exemplary depiction of an out-of-range analyte results user interface 2100 that represents out-of-range analyte monitoring data along a span (or range) 2110 and includes two sub-spans 2112 and 2114 of out-of-range analyte monitoring data. A first sub-span 2110 represents a first out-of-range group of values, and a second sub-span 2114 represents a second out-of-range group of values. A third sub-span 2116 is also shown and conveys to the user that moving below the first sub-span moves the user into an in-range value.

In one variation, and as shown in FIG. 20 and FIG. 21, the analyte monitoring device 110 is measuring glucose, and the mobile application is displaying glucose data. In the examples shown in FIG. 20 and FIG. 21, the spans include a range of glucose values divided into in-range values and out-of-range values where the out-of-range values may be above range values. In the example variation in FIG. 20, the first sub-span representing the first in-range group of values (e.g., 70 mg/dL to 125 mg/dL) is marked with an icon 2020 to indicate that the user's glucose level falls within the corresponding first sub-span. In the example variation in FIG. 21, the second sub-span representing the second out-of-range group of values (e.g., 300 mg/dL to 400 mg/dL) is marked with an icon 2120 to indicate that the user's glucose level falls within the corresponding second sub-span. Although glucose is shown as the analyte being measured, the mobile application can be adapted to other analytes.

In the variations shown in FIG. 20 and FIG. 21, the icons are boxes that fill a majority portion of the respective sub-spans. Other types of indicators, symbols, or markings may be provided. In some variations, the lengths of the sub-spans may vary from each other and may represent different amounts of the range. In some variations, the lengths of the sub-spans may be the same as each other but may vary in the amount of the ranges represented (e.g., the length of the sub-spans may not be to scale). In some variations, additional granularity is provided by incorporating additional sub-spans. In some variations, one of the in-range analyte results user interface and the out-of-range analyte results user interface may have more granularity than the other.

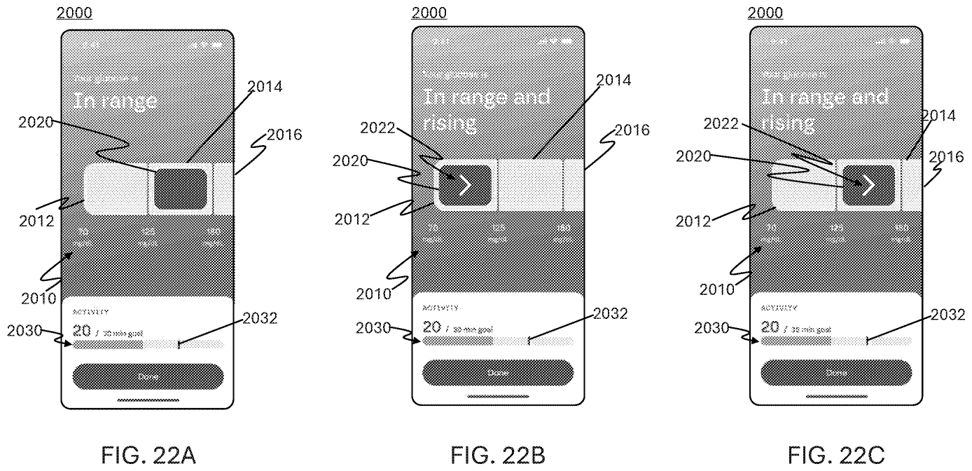

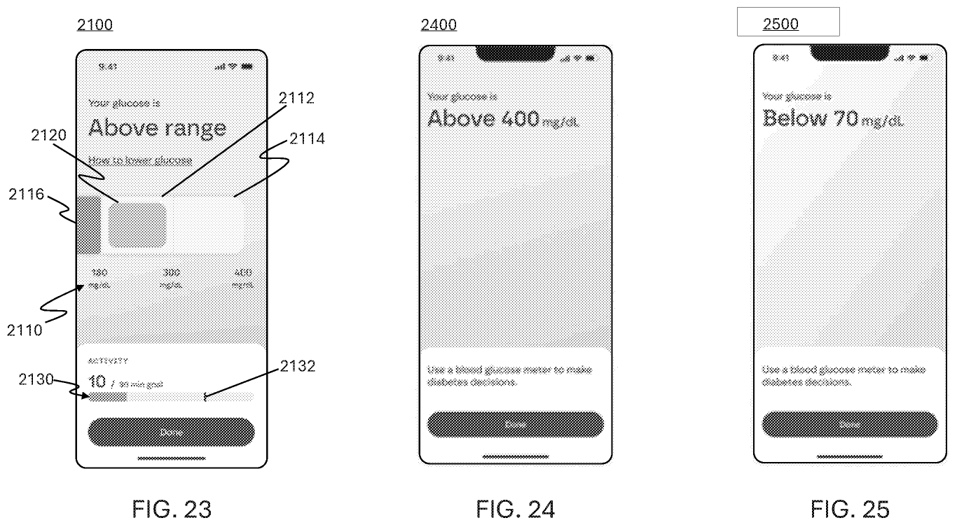

FIGS. 22A, 22B, and 22C show variations of the in-range analyte results user interface 2000, and FIG. 23 shows variations of the out-of-range results user interface 2100. In addition to the icon indicating in which range the user's analyte level is at a given time, a secondary icon may be provided to convey additional information about the user's analyte level. For example, the secondary icon may indicate changes, rates, or other details related to the analyte level. As shown in FIG. 22B in an example variation of the in-range analyte results user interface 2000, a secondary icon 2022 is an arrow that overlies the icon 2020 to indicate that the user's analyte level is rising. Arrows and other symbols as well as alphanumeric values, numbers, and text may be used as the secondary icon 2022. The secondary icon 2022 may overlie or be adjacent to the icon.

Also provided, in some variations, the in-range results user interface and the out-of-range results user interface may provide insights (or messages) to provide educational information or other data as described herein. The in-range results user interface and the out-of-range results user interface may also include supplementary data views on a portion of the user interface. The supplementary data view may include activity data, sleep data, meal data, medication data, or other data associated with the user. In the variation shown in FIG. 20 and FIGS. 22A to 22C, a fillable activity data bar 2030 is provided on the in-range analyte results user interface, and in the variation shown in FIG. 21 and FIG. 23, a fillable activity data bar 2130 is provided on the out-of-range analyte results user interface, to convey the amount of activity that the user has engaged in over a predefined time period, such as the current day. An amount of which the fillable activity data bar is filled represents the amount of activity that the user has engaged in. An end point of the fillable activity data bar may represent an activity goal of the user. In some variations, a marker 2032 and 2132 that overlies the fillable activity data bar 2030 and 2130, respectively, may represent the activity goal of the user.

FIG. 24 and FIG. 25 show variations of beyond reporting range user interfaces 2400 and 2500, respectively. In some variations, the analyte monitoring device 110 may report analyte levels within a certain range of values. If an analyte level is determined to be above the range of reportable values, the mobile application may render the user interface 2400 to report the above range value and provide information (via an insight) on next steps for the user to take. Similarly, if an analyte level is determined to be below the range of reportable values, the mobile application may render the user interface 2500 to report the below range value and provide information, via an insight, on next steps for the user to take.

The mobile application may include a home user interface that is presented when the user selects or opens the mobile application on the mobile computing device 102. In some variations, the home user interface is also accessible through other user interfaces provided by the mobile application. In some variations, the home user interface may provide an overview of the wear session of the analyte monitoring device 110. The home screen may also include other data, such as the rest data, activity data, medication data, or food or meal data. An insight related to the wear session of the analyte monitoring device 110 or related to a recent scan or upcoming event may also be provided on the home user interface.

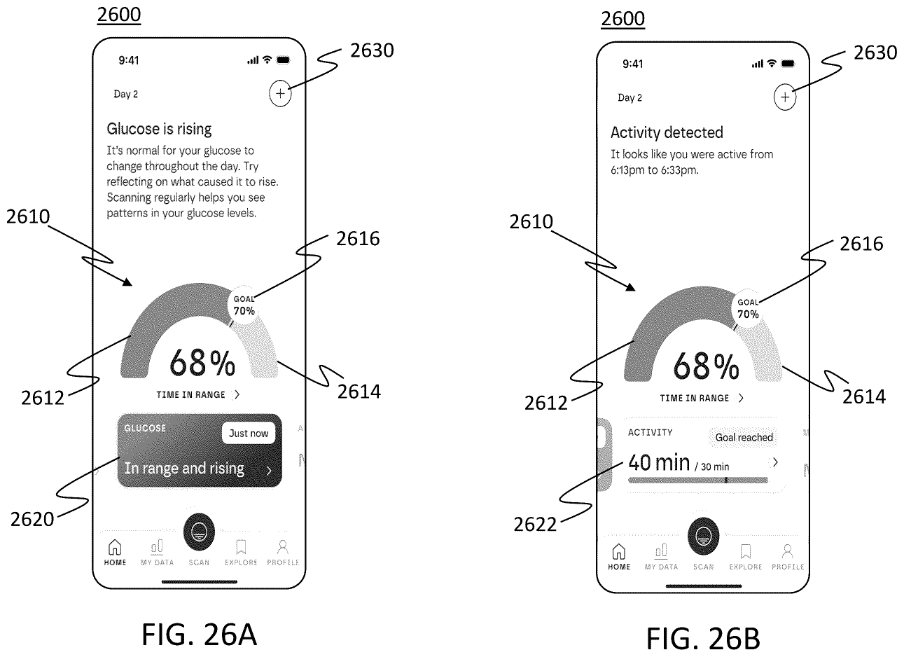

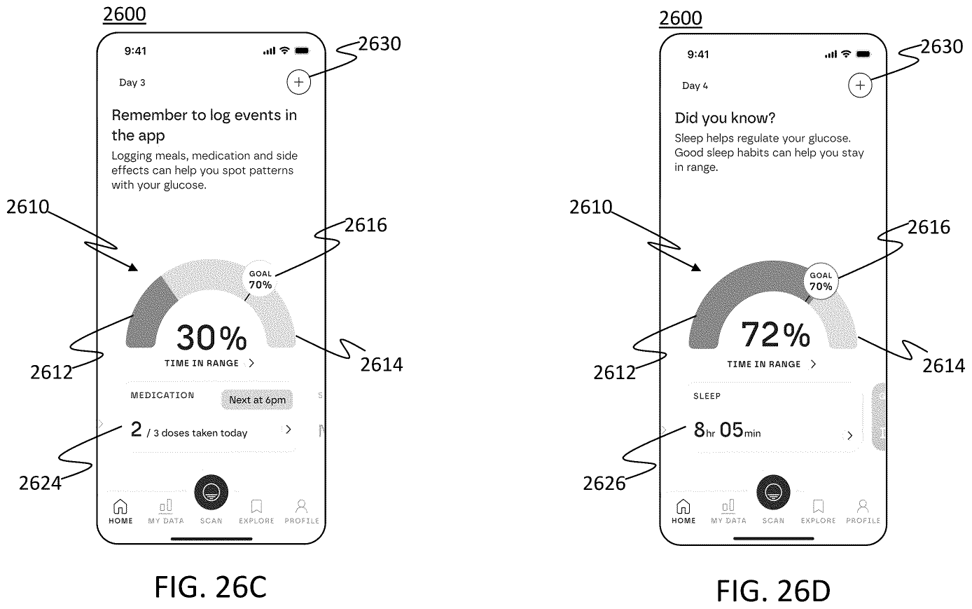

FIG. 26A to FIG. 26D are exemplary depictions of a home user interface 2600 according to a variation. As shown, the home user interface 2600 generated by the mobile application includes a time in-range gauge 2610 that provides details regarding a percentage in which the user has in-range analyte monitoring data and a percentage in which the user has out-of-range analyte monitoring data, determined based on the analyte monitoring data received from the analyte monitoring device 110. In some variations, the time in-range gauge is updated each time analyte monitoring data is received from the analyte monitoring device 110. In some variations, the percentage is based on the analyte data from the current day.

The time in range gauge 2610 is a segment formed of two sections, a first section 2612 representing a percentage of in-range analyte monitoring data and a second section 2614 representing a percentage of out-of-range analyte monitoring data. The first section 2612 may be shaded or colored in a first color to represent the in-range percentage, while the second section 2614 is shaded or colored in a second color to represent the out-of-range percentage. In some variations, rather than or in addition to being color-coded, the time in-range gauge may incorporate one or more of patterns, symbols, alphanumeric values, numbers, and text to distinguish between the percentage of in-range analyte monitoring data and the percentage of out-of-range analyte monitoring data. In some variations, a line or demarcation is provided to distinguish between and separate the percentage of in-range analyte monitoring data and the percentage of out-of-range analyte monitoring data. In some variations, such as that shown in FIGS. 26A-26D, the time in range gauge 2610 is an arc, and the first section 2612 is first portion of the arc while the second section 2614 is a remaining or second portion of the arc. In other variations, the time in range gauge 2610 may be other shapes broken into or separating the first section 2612 and the second section 2614 to represent the in-range and out-of-range percentages.

Overlying the time in-range gauge may be a marker 2616 representing a time-in-range goal of the user. The marker 2616 may be one or more of a line, a symbol, and a demarcation to convey to the user if the time in-range goal has been satisfied. In some variations, the time in-range gauge represents a daily time in-range. In some variations, the time in-range gauge represents a wear session time in-range. In some variations, other time periods may be used. For example, the user may wish to know the time in-range over the past 48 hours and may set the time in-range gauge accordingly through one or more selectable options or user interface controls.

The home user interface 2600 also includes, in some variations, scrollable cards with additional data or supplemental information, each of which, once selected, transition to a user interface or window that provides more information and options about the selected card.

An activity card 2622, as shown in FIG. 26B, is provided to convey the amount of activity that the user has engaged in over a predefined time period, such as the current day. Similar to the supplementary data view described with reference to the analyte results user interface, the activity card may include a fillable activity data bar. An amount of which the fillable activity data bar is filled represents the amount of activity that the user has engaged in. An end point of the fillable activity data bar may represent an activity goal of the user. In some variations, a marker that overlies the fillable activity data bar may represent the activity goal of the user. In some variations, the fillable activity bar and marker dynamically adjusts based on the amount of activity of the user.

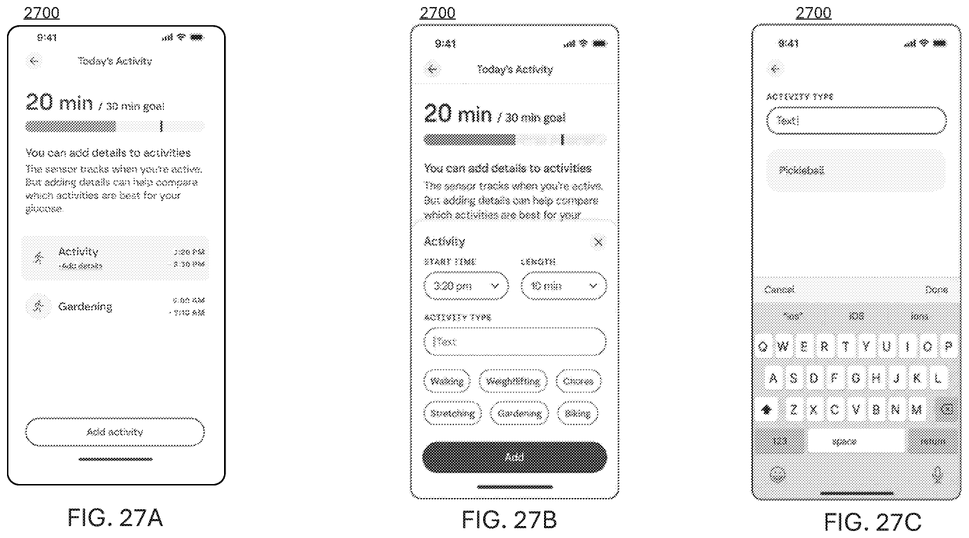

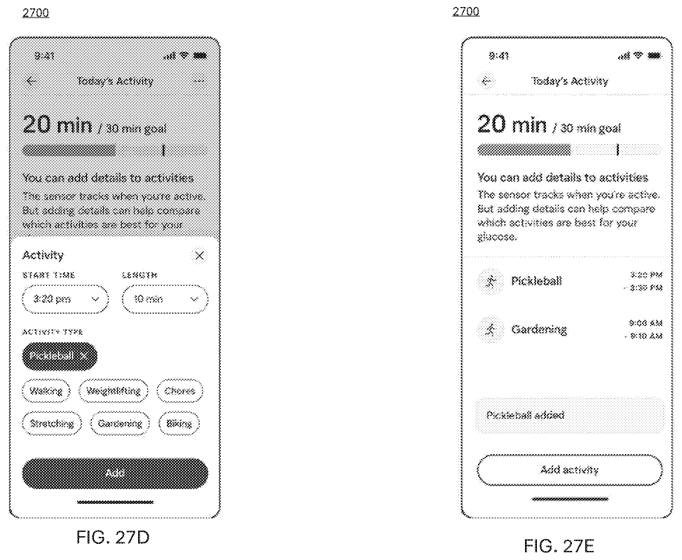

Selection of the activity card 2622 from the home user interface 2600 or selection of a “+” button 2630 when the activity card 2622 is shown on the home user interface 2600 may result in transitioning to an activity logging user interface 2700, aspects of which are shown in FIG. 27A through FIG. 27E. The activity logging user interface 2700 transitions through a series of screens to allow the user to enter data around one or more activities, resulting in creation of activity intervals. Activity intervals may also be determined based from the activity data received from the analyte monitoring device 110, as further described herein. The activity logging user interface 2700 provides user interface controls to adjust data of one or more logged activities. For example, the user may adjust one or more of the length of time, start time, stop time, or the type of activity. The activity logging user interface 2700 additionally allows the user to manually add activities, such as biking, that may not be detected by the user's analyte monitoring device 110.

A medication card 2624, as shown in FIG. 26C, from the home user interface 2600, provides an overview of medication taken by the user. In some variations, the medication information is that of medication related to or impacted by the analyte being measured by the analyte monitoring device 110; for example, diabetes-related medication for an analyte monitoring device that measures glucose levels. The medication card may include an indication of number of doses taken, remaining doses, doses skipped or missed, and a time at which medication needs to be taken.

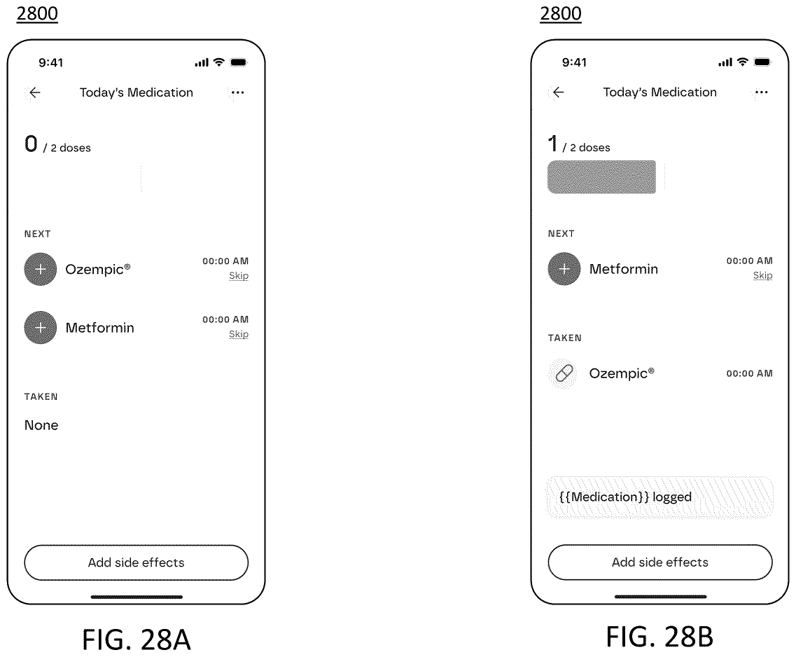

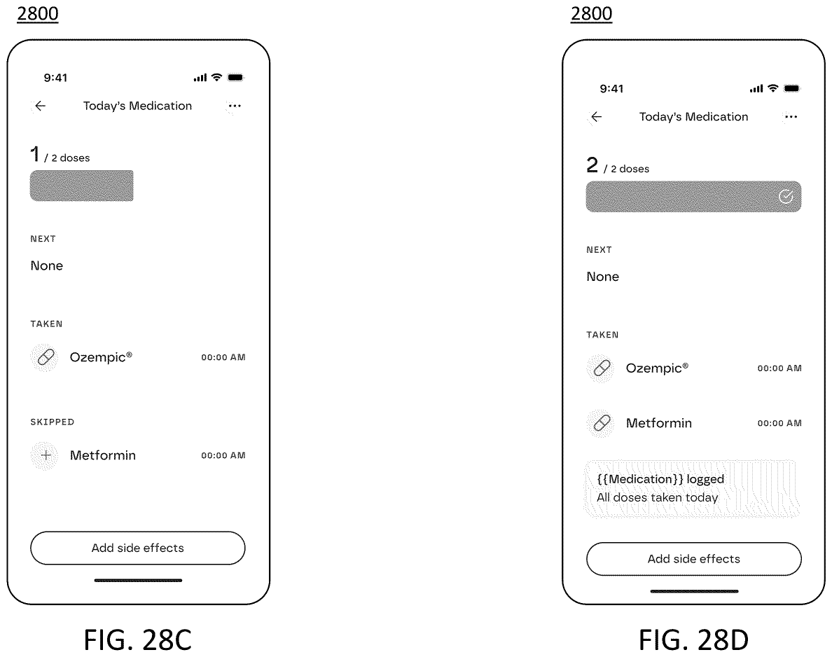

Selection of the medication card from the home user interface or selection of the “+” button 2630 when the medication card 2624 is shown on the home user interface 2600 may result in transitioning to a medication logging user interface 2800, aspects of which are shown in FIGS. 28A through 28D. The medication logging user interface includes details about the medications taken or scheduled to be taken, such as the medication type and scheduled time. The medication logging user interface also provides user interface controls for inputting or editing details related to the medication, such as side effects, time the medication is taken, and removing or skipping a medication dose.

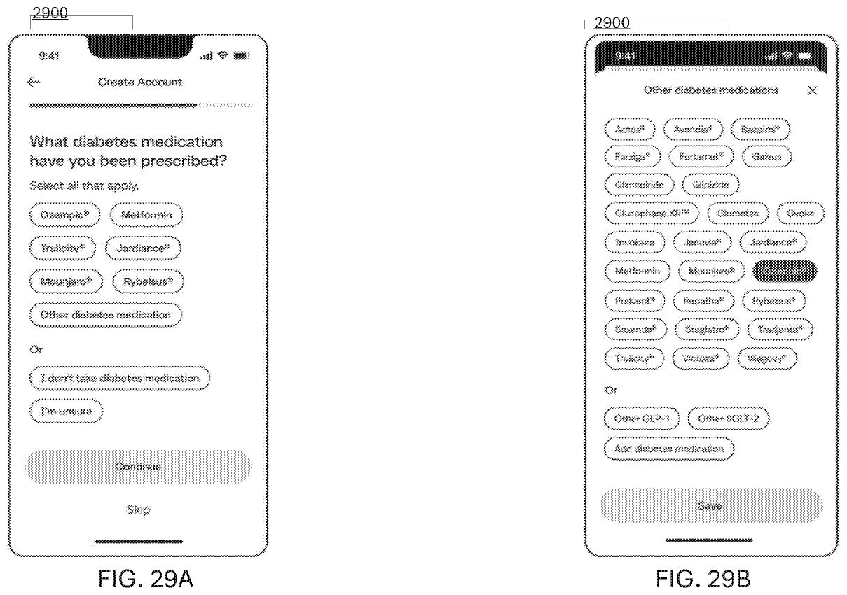

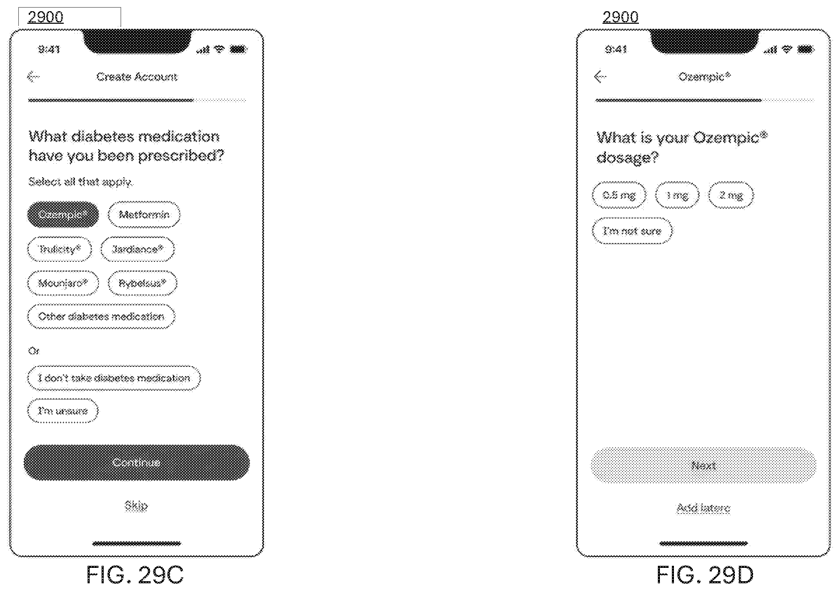

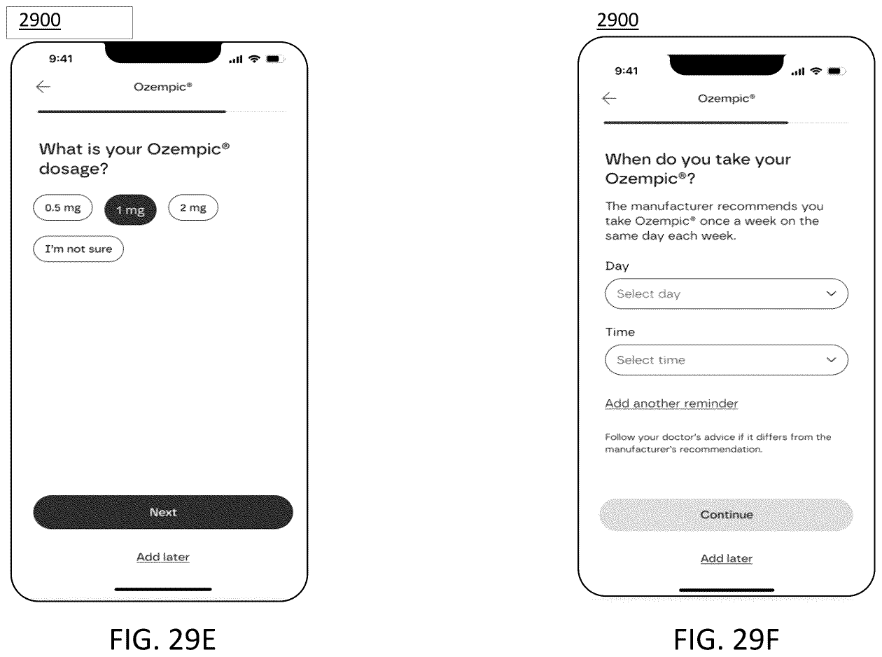

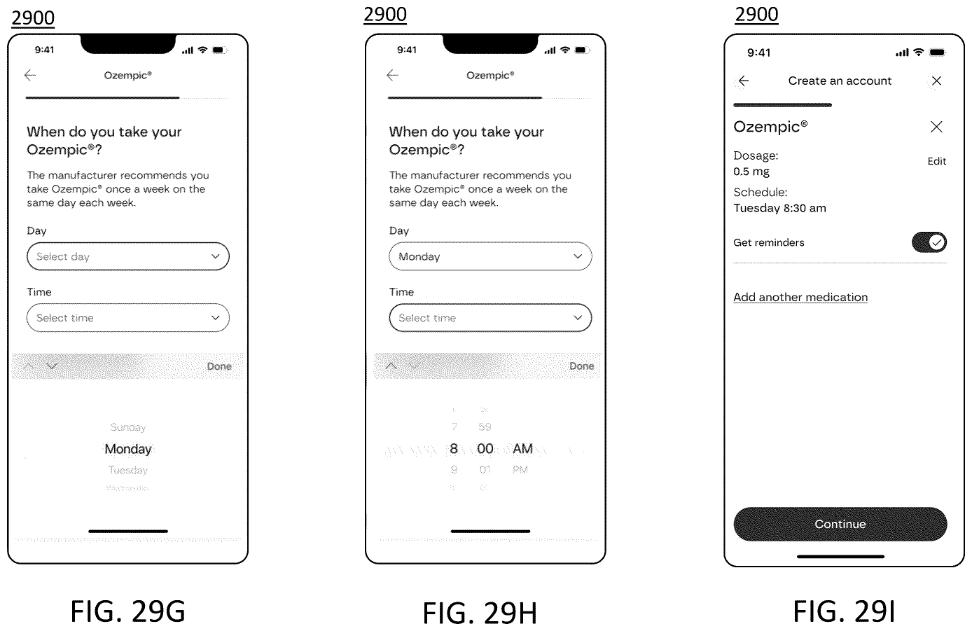

In some variations, a medication registration user interface 2900, aspects of which are shown in FIG. 29A and FIG. 291, is provided by the mobile application to allow the user to set a schedule for taking medications, which may include entering data related to medications and dosages. The medication registration user interface 2900 may be provided at or prior to the beginning of use of the analyte monitoring device 110 and the mobile application (e.g., prior to a wear session of the analyte monitoring device 110). The medication data entered may then be populated in the medication logging user interface 2800.

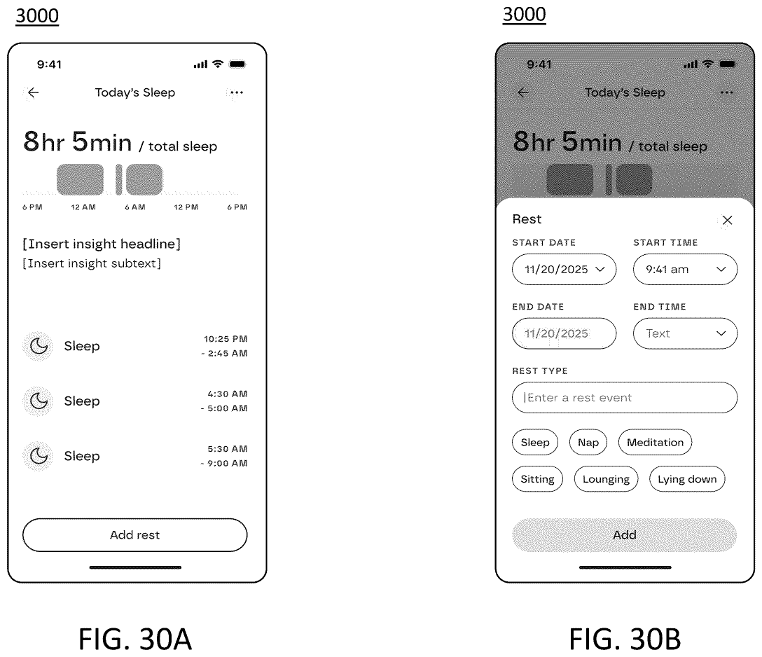

A sleep card 2626, as shown in FIG. 26D, from the home user interface, provides an overview of the amount of time of sleep that the user has had over the course of a time period, such as a daily view of time slept. Selection of the sleep card from the home user interface 2600 or selection of the “+” button 2630 when the sleep card 2626 is shown on the home user interface 2600 may result in transitioning to a sleep logging user interface 3000. FIG. 30A and FIG. 30B depict aspects of the sleep logging user interface 3000 according to some variations. The sleep logging user interface 3000 includes a fillable sleep data bar that indicates sleep intervals with a start time and an end time. A summary of data related to the sleep intervals may also be provided.

The sleep logging user interface 3000 allows for the user to enter data, including dates, times, and rest events or types, for one or more sleep intervals, resulting in creation of sleep intervals. Sleep intervals may also be determined based from the activity, light, and time data received from the analyte monitoring device 110, as further described herein. The sleep logging user interface 3000 also provides user interface controls to adjust data of one or more sleep intervals. For example, the user may adjust the length of time, the start time, and the end time. The sleep logging user interface 3000 additionally allows the user to manually add types of rest beyond sleep, such as meditation or a nap, that may not be detected by the user's analyte monitoring device 110.

An analyte card 2620, on the home user interface 2600 as shown in FIG. 26A, provides an overview of the user's current analyte level or most recent analyte level. The analyte card 2620 may also provide information on the status of the analyte monitoring device 110, such as, for example, when the analyte monitoring device 110 is adjusting and/or analyte data is not available, when the wear session has ended, or when it's time for the final scan (e.g., data transfer) of the analyte monitoring device 110. For example, the overview may include one or more of a range of the analyte level, a category of the analyte level (e.g., in-range data, out-of-range data, above range data, or below range data), and a time at which the analyte level was received.

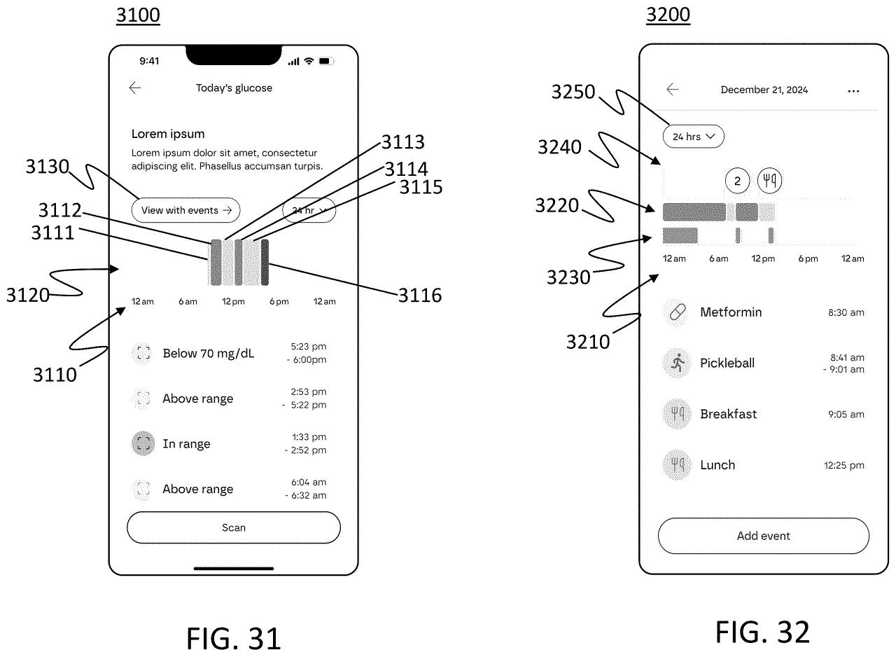

Selection of the analyte card 2620 or selection of the “+” button 2630 when the analyte card 2620 is shown on the home user interface 2600 causes the mobile application to transition to an analyte timeline user interface 3100 as shown in FIG. 31. The analyte timeline user interface 3100 includes a timeline 3110 extending across a temporal span with one or more segments aligned in a first row 3120 along the timeline and representing analyte monitoring data. In the variation shown in FIG. 31, segments 3111, 3112, 3113, 3114, 3115, and 3116 are shown. In some variations, each segment is representative of a category of analyte monitoring data, such as in-range data, out-of-range data, above range data, or below range data, and is aligned with the timeline 3110 to indicate the temporal span of the respective category of analyte monitoring data. Each segment may be a coded or labeled data block to signify the category and the temporal span of analyte monitoring data. In some variations, each segment is color-coded. In some variations, each segment incorporates a label or symbol to indicate the category. The temporal span may present a daily view (e.g., the current day over a 24-hour window) of the analyte monitoring data, although other periods of time may be implemented. The first row 3120 may be scrollable to allow the user to scroll to earlier times not originally shown and to scroll back to the original view.

The analyte timeline user interface 3100 may also include, separate from the timeline, a summary of the analyte monitoring data. For example, one, two, three, four or more of the most recent segments of analyte monitoring data may be listed with a respective category and time interval.

The analyte timeline user interface 3100 may transition to an analyte and event timeline user interface 3200 upon selection of a button or icon, for example, a view with events button 3130. FIG. 32 is an exemplary depiction of an analyte and event timeline user interface 3200 according to a variation. In addition to one or more segments aligned in a first row 3220 along a timeline 3210 and representing analyte monitoring data (similar to the analyte timeline user interface 3100 shown in FIG. 31), one or more second segments are aligned in a second row 3230 along the timeline 3210 and represent one or more activity intervals of the user and/or one or more third segments are aligned in the second row 3230 along the timeline 3210 and represent one or more sleep intervals of the user. The second and third segments represent discrete, noncontinuous traces of activity and sleep, respectively, that are separated by a temporal gap over a portion of the timeline 3210 over which the analyte monitoring data is represented. The second and third segments may be distinguished from one another by incorporation of a respective color representing activity segments and a respective color representing sleep segments. In some variations, symbols, icons, or patterns are used to distinguish between the second and third segments. The analyte and event timeline user interface 3200 may include one or more user interface controls configured to selectively display or hide one or more categories of physiological information including the analyte monitoring data, the activity intervals, or the sleep intervals.

The analyte and event timeline user interface 3200 may include a third row 3240 in which one or more event icons are aligned along the timeline and represent a respective event. The one or more event icons represent discrete events that are separated by a temporal gap over the portion of the timeline over which the analyte monitoring data is represented. The one or more event icons may represent a medication dose, side effects, and a meal. If two or more events occur near in time to one another such that display of the two or more event icons is impacted on the third row 3240 (e.g., the two or more event icons overlap one another, making it difficult to discern the events and respective times), a numerical icon is included to denote the presence of multiple events.

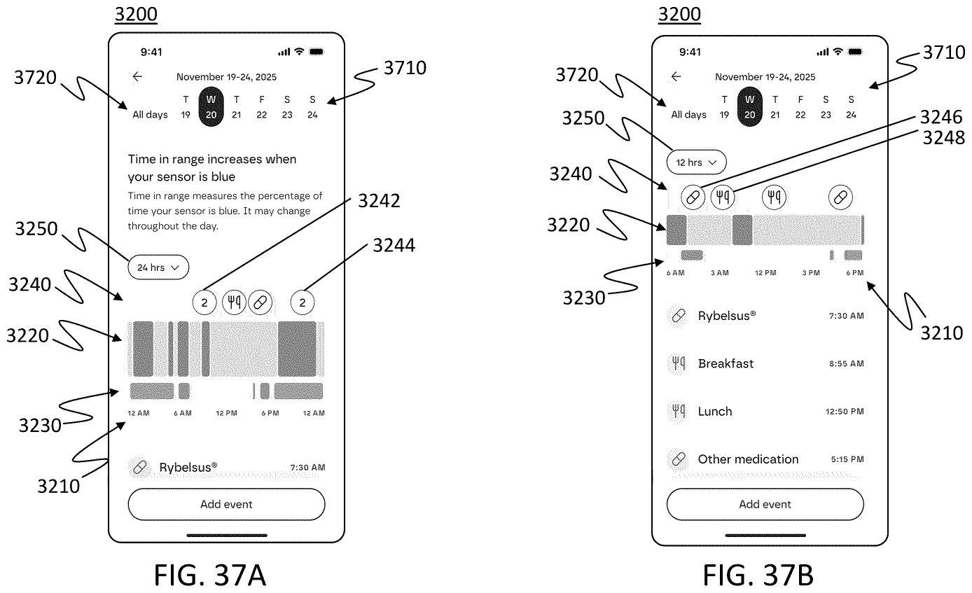

The various timeline user interfaces may allow for selection of a different temporal span to be displayed. For example, a dropdown filter 3250 may provide various options of temporal spans, selection of which results in the temporal span and the timeline being adjusted, further described with reference to FIGS. 37A and 37B.

The analyte and event timeline user interface 3200 may also include, separate from the timeline, a summary of the events. For example, one, two, three, four or more of the most recent events may be listed with a respective time interval and a respective event icon.

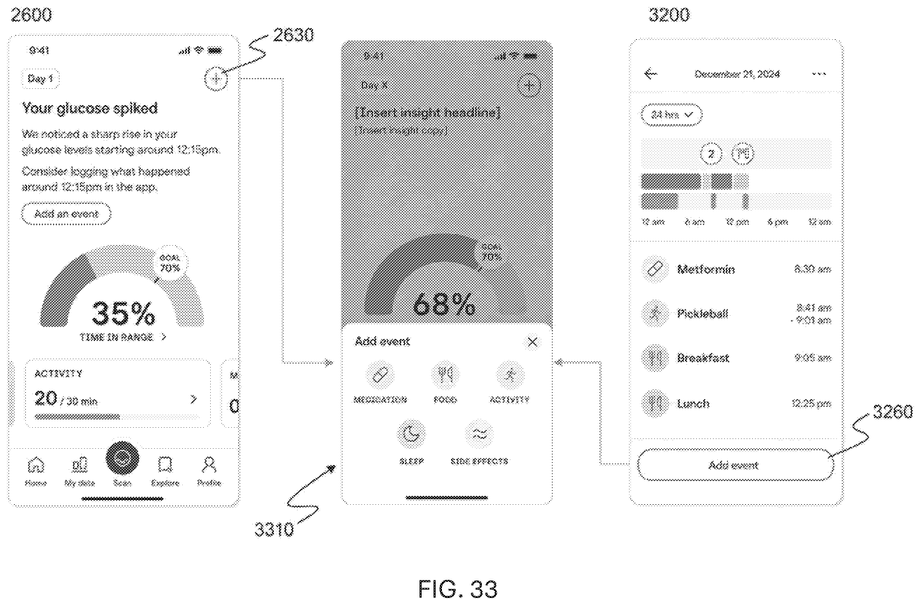

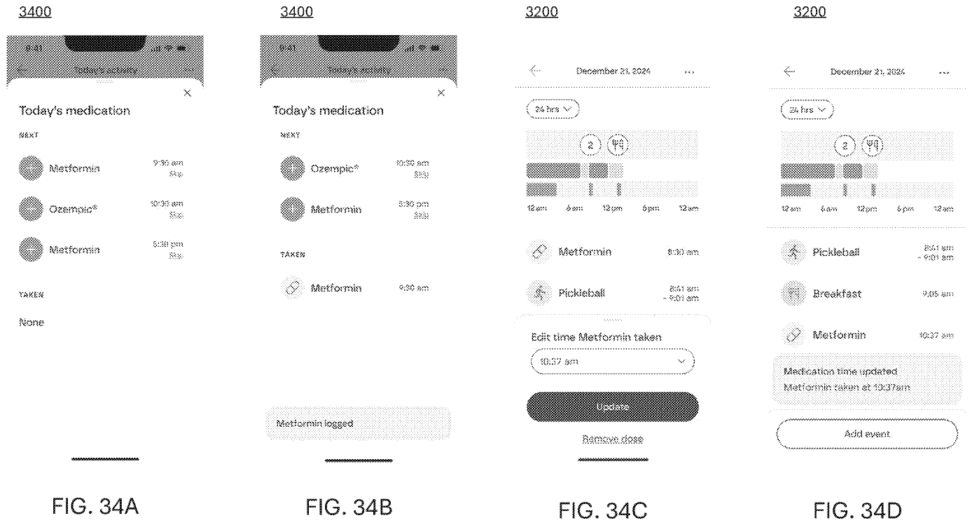

Various user interfaces provided by the mobile application allow for the entry and/or modification of events. For example, selection of an icon or button may generate a window that allows for selection of an event. As an example, the user may select the “+” button 2630 or a first add event button 2640 on the home user interface 2600 or a second add event button 3260 on the analyte and event timeline user interface 3200, as shown in FIG. 33, which renders a window 3310 with event icons that may be selected. In some variations, selection of a medication icon results in transition to the medication logging user interface 2800, selection of an activity icon results in transition to the activity logging user interface 2700, selection of a sleep icon results in transition to the sleep logging user interface 3000, selection of a side effects button results in transition to a side effect logging user interfaces 3500, and selection of a food icon results in transition to a food logging interface 3600. In some variation, selection of one of the event icons results in transition to a quick logging user interface 3400, an example of aspects of which are shown in FIGS. 34A and 34B. The quick logging user interface 3400 may provide one or more user interface controls for adding or modifying an event including the event type and time. FIGS. 34C and 34D depict example representations on how the addition or modification is shown on the analyte and event timeline user interface 3200.

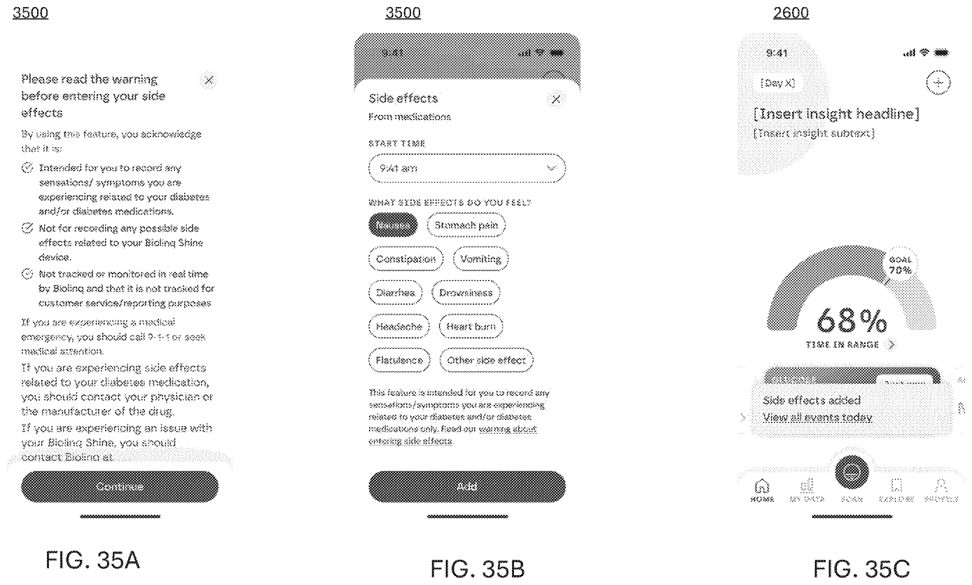

The side effects logging user interface 3500 provides user interface controls for the user to enter data regarding one or more side effects, aspects of which are shown in FIGS. 35A through 35 C. A warning screen may provide information on intention and limitations of the side effects logging tool and may include a user acceptance button. Through the side effects logging user interface 3500, the user is able to provide details regarding side effects, including time and medication and selection of side effects. FIG. 35C depicts an example representation of how the side effects logging is shown on the home user interface 2600.

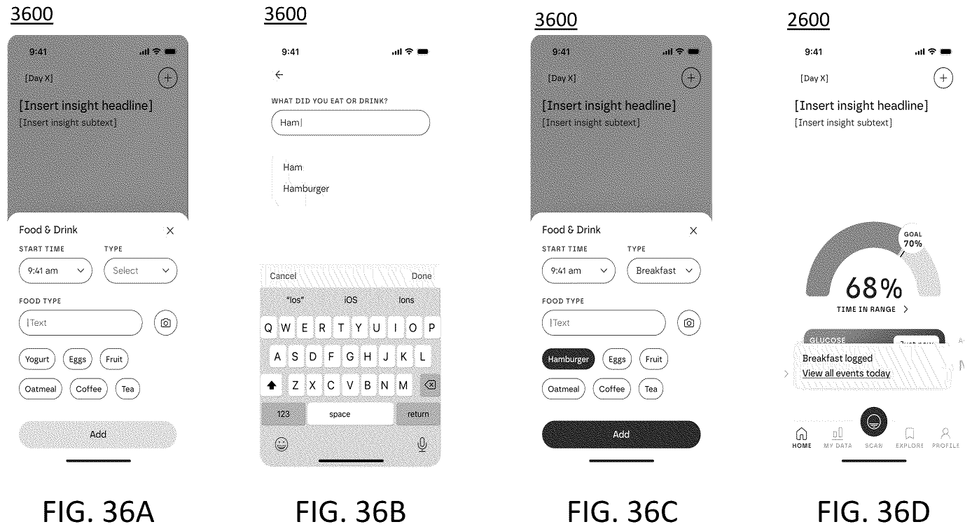

The food logging user interface 3600 provides user interface controls for the user to enter data regarding one or more meals. FIGS. 36A through 36D provides exemplary depictions of a series of the food logging user interface 3600 that includes text entry fields and selection tools for entering foods eaten, type of meal, and time. FIG. 36D depicts an example representation of how the food logging is shown on the home user interface 2600.

FIGS. 37A and 37B provide additional depictions of the analyte and event timeline user interface 3200, which may also be accessed via the home user interface 2600 by selection of a data icon or selection of the analyte card. FIG. 37A shows a variation of the analyte and event timeline user interface 3200 in which the timeline portion is scrolled down to display at least a portion of a calendar 3710. The calendar 3710 allows for user selection of a particular day or all days (an “all days” button 3720) of the wear session of the analyte monitoring device. The dropdown filter 3250 provides various options of temporal spans, such as 24 hours, 12 hours, and six hours, selection of which results in the temporal span and the timeline being adjusted to display the selected temporal span. When selecting a smaller temporal span, additional details or more detailed, precise views may be generated in the rows. For example, with a 24-hour temporal span, as shown in FIG. 37A, two event icons overlie each other, denoted by the numerical icons 3242 and 3244 in the third row 3240. With selection of the 12-hour temporal span, however, as shown in FIG. 37B, separate event icons 3246 and 3248 are generated from the numerical icon 3242 and displayed accordingly with respect to the timeline 3210, allowing the user to see a more granular view of the events with respect to the timeline 3210. The user can scroll left and right to see additional granular details.

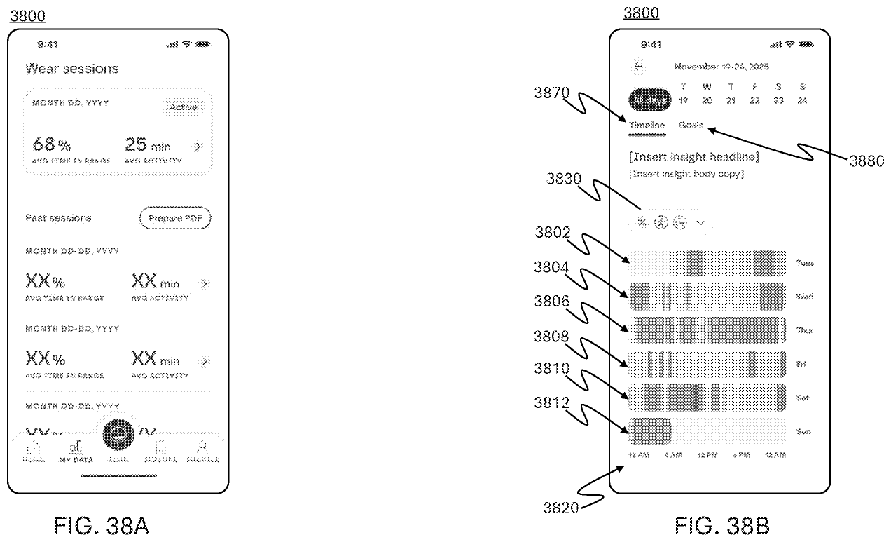

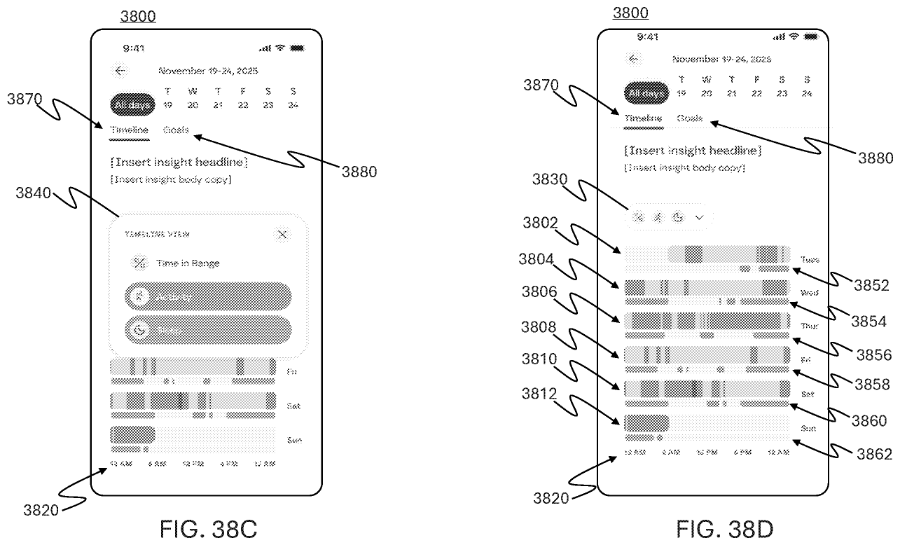

A wear session analyte timeline user interface 3800 provides a timeline view over the length of a wear session of the analyte monitoring device 110 and may be viewed by selecting the “all days” button 3720 in the calendar 3710 of the analyte and event timeline user interface 3200. FIGS. 38A through 38D provide exemplary depictions of variations of the wear session analyte timeline user interface 3800. FIG. 38A depicts a variation in which a summary of the current wear session and summaries of past wear sessions are provided. Selection of the current wear session summary may result in the mobile application rendering the variation shown in FIG. 38B. In some variations, the summary view is bypassed.

As shown in FIG. 38B, the wear session analyte timeline user interface 3800 includes a plurality of rows 3802, 3804, 3806, 3808, 3810, and 3812, each row representing a particular day of the wear session. In some variations, additional or fewer rows are provided, depending on the operational length of use of the analyte monitoring device 110. In some variations, the number of rows shown may be equal to the number of days that have so far elapsed in the wear session. For example, on the fourth day of wear, four rows are shown. At the start of the fifth day, an additional row is added to reflect the current, fifth day.

One or more segments are aligned in each row along a timeline 3820 extending across a temporal span to represent daily analyte monitoring data from the analyte monitoring device 110. In some variations, each segment is representative of a category of analyte monitoring data, such as in-range data, out-of-range data, above range data, or below range data, and is aligned with the timeline to indicate the temporal span of the respective category of analyte monitoring data. Each segment may be a coded or labeled data block to signify the category and the temporal span of analyte monitoring data. In some variations, each segment is color-coded. In some variations, each segment incorporates a label or symbol to indicate the category. For an incomplete wear session, one or more of the plurality of rows will not include segments to signify that the analyte monitoring device 110 has not collected data for the particular days.

As shown in FIG. 38C, selection of the dropdown filter 3830 provides a selection window 3840 to allow for the user to select one or more of analyte data, activity data, and sleep data. As shown in FIG. 38D, upon selection of analyte data, activity data, and sleep data, activity intervals and sleep intervals are populated in secondary rows 3852, 3854, 3856, 3858, 3860, and 3862 positioned beneath each of the plurality of rows of analyte data to display the activity intervals and the sleep intervals. In particular, one or more second segments are aligned in a secondary row along the timeline 3820 and represent one or more activity intervals of the user and/or one or more third segments are aligned in the secondary row along the timeline and represent one or more sleep intervals of the user. The second and third segments represent discrete, noncontinuous traces of activity and sleep, respectively, that are separated by a temporal gap over a portion of the timeline over which the analyte monitoring data is represented. The one or more second segments and the one or more third segments may be color coded to allow for the user to distinguish between the two segments in the secondary rows. For example, a first color may represent activity and a second color may represent sleep.

A data goals user interface 3900 provides a bar graph depiction of the user's data with respect to goals over a wear session of the analyte monitoring device 110. The user may toggle between the timeline view and the data goals view by selecting one of a timeline button 3870 or a goals button 3880 in the wear session analyte timeline user interface and the data goals user interface 3900.

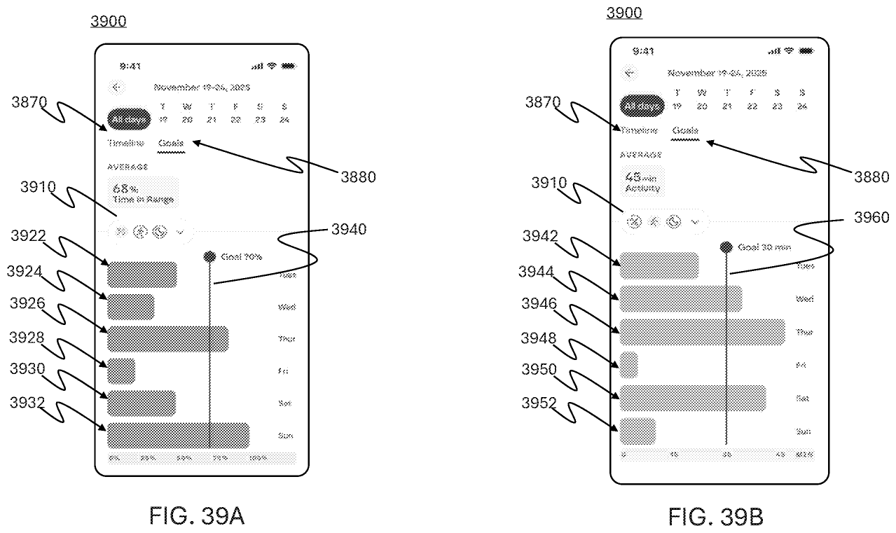

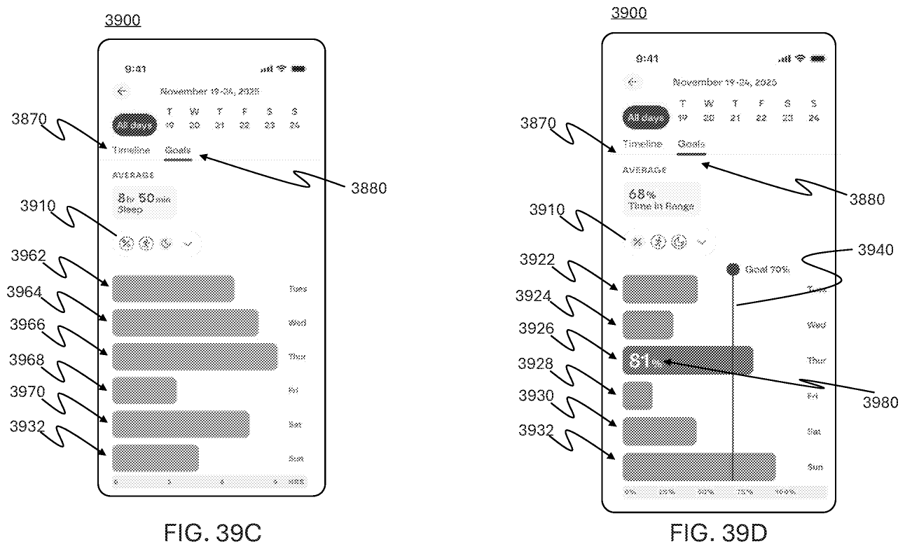

FIGS. 39A through 39D are exemplary depictions of variations of the data goals user interface 3900. A dropdown filter 3910 provides for selection of the type of data to be displayed, including, for example, time in-range (or analyte) data, activity data, sleep data, or combinations thereof. A plurality of data bars is arranged in rows, with each data bar representing a value of a respective type of data for a particular day of a wear session of the analyte monitoring device 110. In one variation, as shown in FIG. 39A, analyte data bars 3922, 3924, 3926, 3928, 3930, and 3932 represent daily percentage of time in-range and are compared against a time in-range goal 3940, denoted by a line along the data bars to signify the user's progress towards the goal. In one variation, activity data bars 3942, 3944, 3946, 3948, 3950, and 3952 represent daily activity time, an example of which is shown in FIG. 39B, which may be compared against an activity goal 3960, denoted by a line along the data bars to signify the user's progress towards the goal. In one variation, sleep data bars 3962, 3964, 3966, 3968, 3970, and 3972 represent daily amount of time slept, as shown in FIG. 39C. In some variations, as shown in FIG. 39D, selection or tapping of a particular data bar results in an icon 3980 overlying the data bar to provide more granular data regarding the data bar. In some variations, the more granular data is a numerical value.

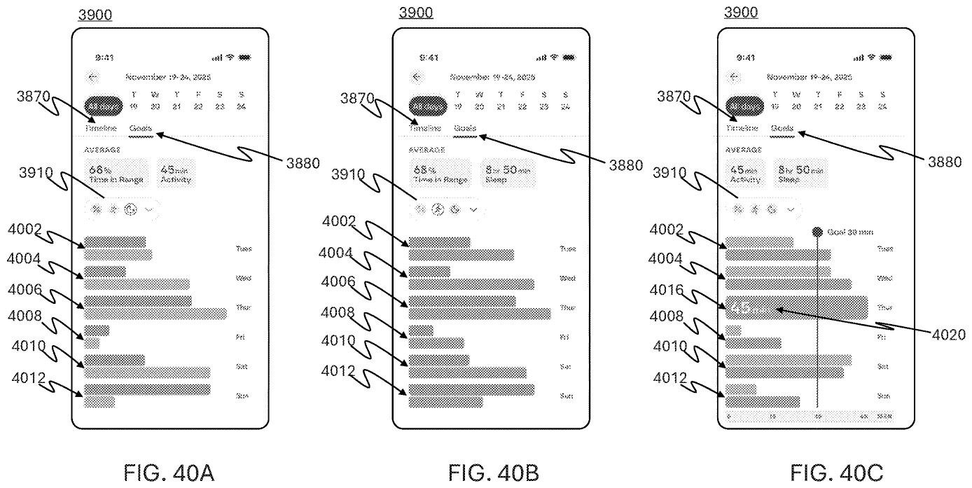

FIGS. 40A through 40C are exemplary depictions of variations of the data goals user interface 3900 in which two types of data are selected for display through the dropdown filter 3910. In this variation, a grouping of two data bars 4002, 4004, 4006, 4008, 4010, and 4012 is provided for each day to represent the two types of selected data. In some variations and as shown in FIG. 40C, selection or tapping of a particular grouping of two data bars results in a first data bar 4016 of the two data bars overlying both of the two data bars with a first icon 4020 overlying the first data bar in the grouping to provide more granular data regarding the first data bar. The goal associated with the first data bar may be rendered on the user interface 3900 to show the user's progress towards the goal. A subsequent selection of tapping of the particular grouping of two data bars results in a second data bar of the two data bars overlying both of the two data bars with a second icon overlying the second data bar in the grouping to provide more granular data regarding the second data bar.

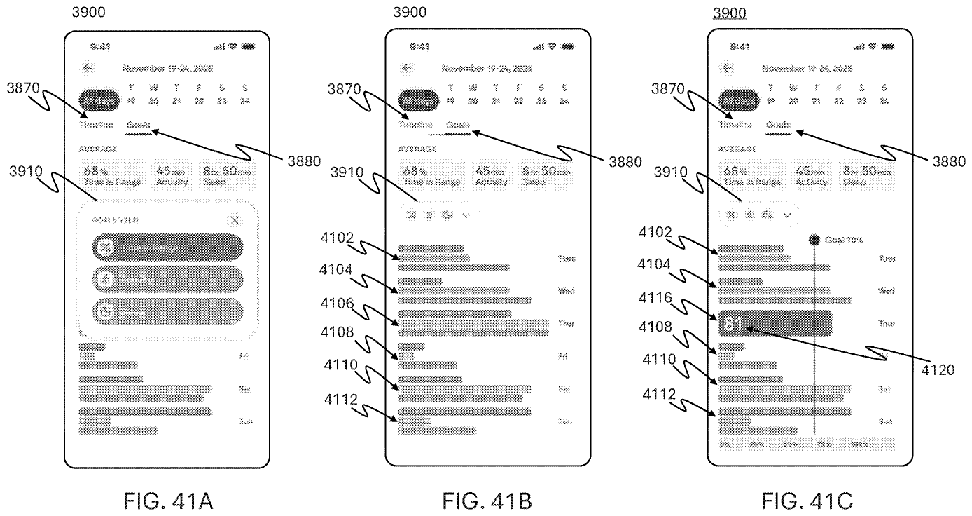

FIGS. 41A through 41C are exemplary depictions of variations of the data goals user interface 3900 in which three types of data are selected for display through the dropdown filter 3910. In this variation, a grouping of three data bars 4102, 4104, 4106, 4108, 4110, and 4112 is provided for each day to represent the three types of selected data. In some variations, as shown in FIG. 41C, selection or tapping of a particular grouping of three data bars results in a first data bar 4116 of the three data bars overlying the three data bars with a first icon 4120 overlying the first data bar in the grouping to provide more granular data regarding the first data bar. The goal associated with the first data bar may be rendered on the user interface 3900 to show the user's progress towards the goal. A subsequent selection of tapping of the particular grouping of three data bars results in a second data bar of the three data bars overlying the three data bars with a second icon overlying the second data bar in the grouping to provide more granular data regarding the second data bar. A subsequent selection of tapping of the particular grouping of three data bars results in a third data bar of the three data bars overlying the three data bars with a third icon overlying the third data bar in the grouping to provide more granular data regarding the third data bar.

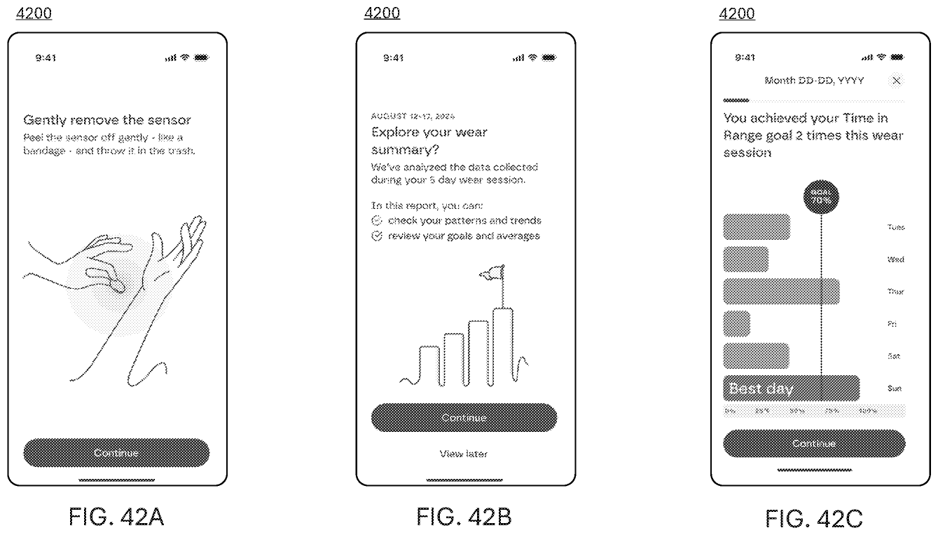

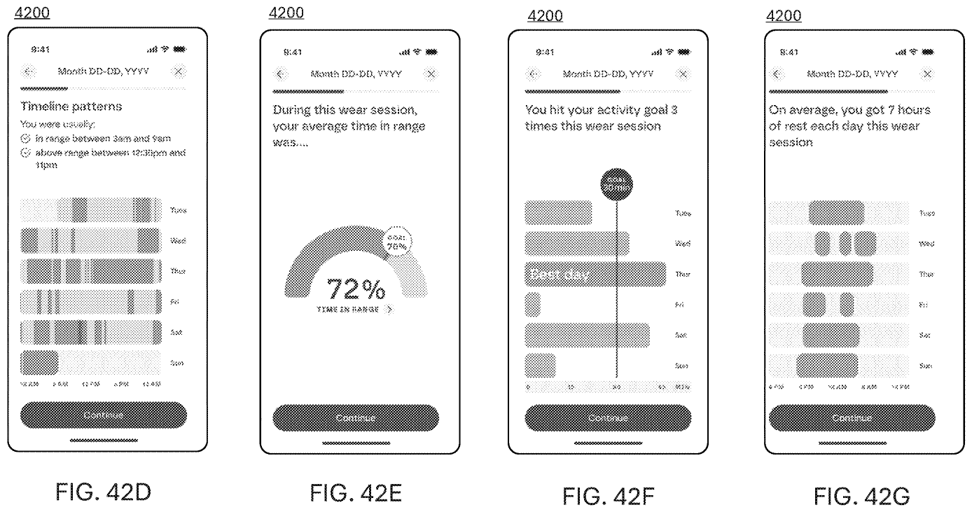

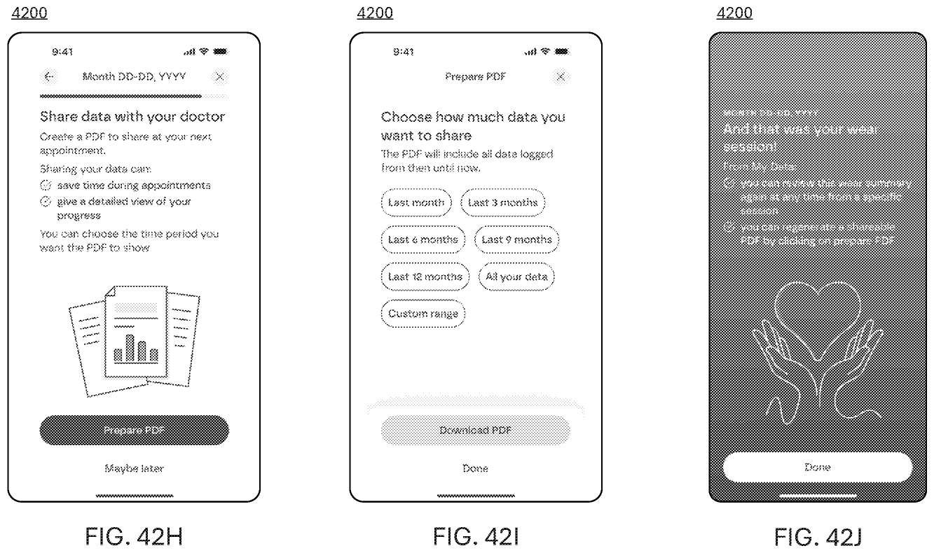

FIGS. 42A through 42J depict aspects of a wear report summary user interface 4200 that may be rendered when the user has completed use of the analyte monitoring device 110. The wear report summary user interface 4200 may provide a series of overviews and summaries relating to the user's use of the analyte monitoring device 110.

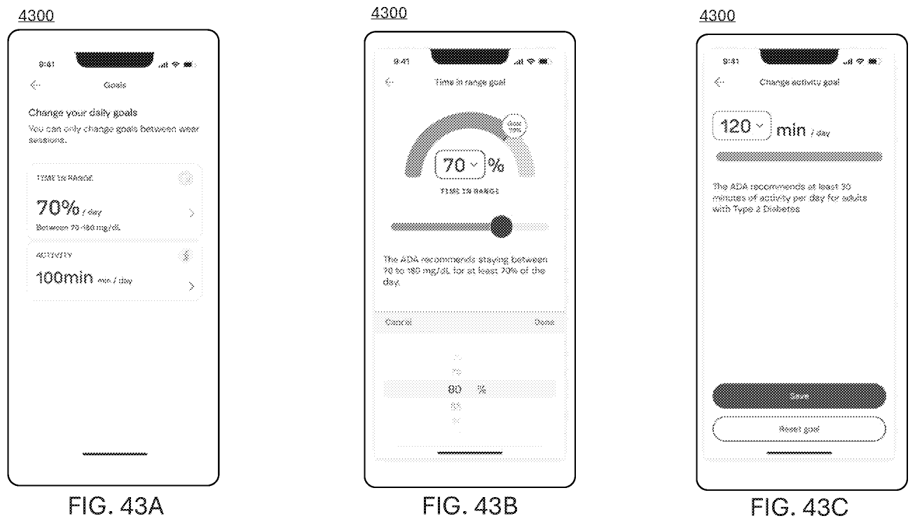

A change goals user interface 4300 provides user interface controls for the user to modify the goals from either predefined goals or those previously set by the user. FIGS. 43A through 43C provides exemplary depictions of a series of the change goals user interface 4300 that provides for adjusting time in-range and activity goals.

The mobile application and rendered user interfaces may employ a color scheme that correlates with the user interface of the analyte monitoring device 110. For example, the LEDs of the analyte monitoring device 110 may utilize a first color to indicate in-range analyte results, a second color to indicate out-of-range analyte results, and a third color to indicate non-reportable or beyond range results. The mobile application may in some variations utilize the first color, the second color, and the third color in the same manner in the rendered user interfaces. In some variations, the first color is blue, the second color is yellow, and the third color is red.

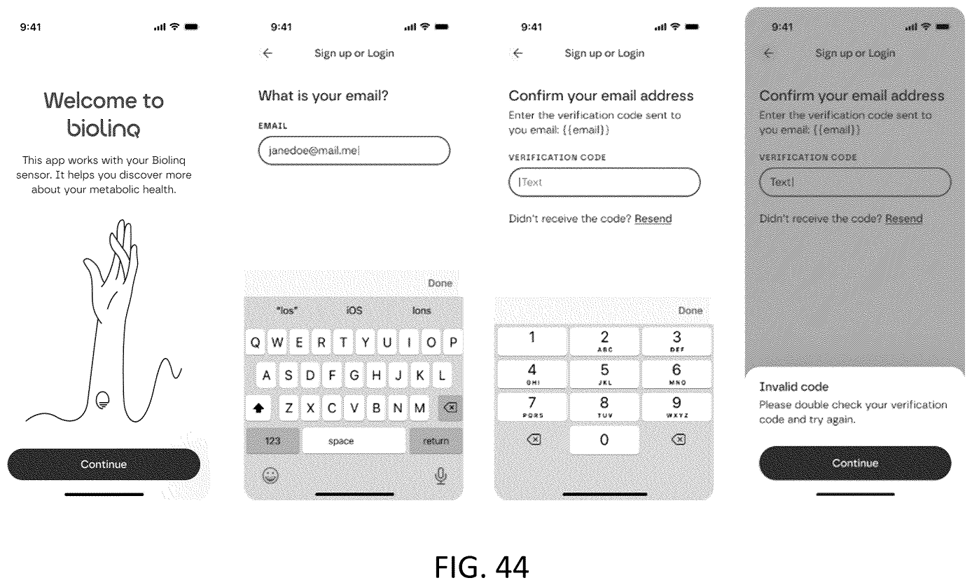

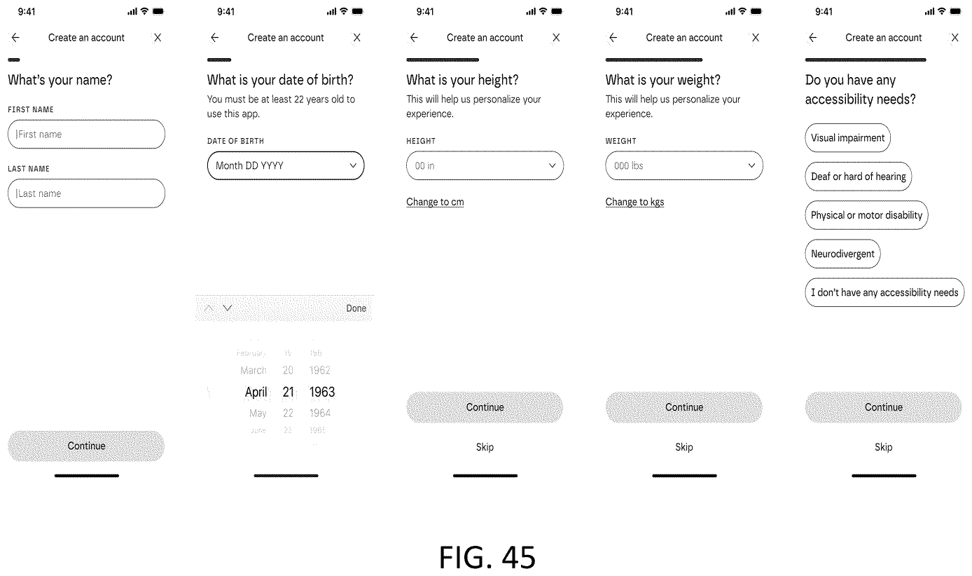

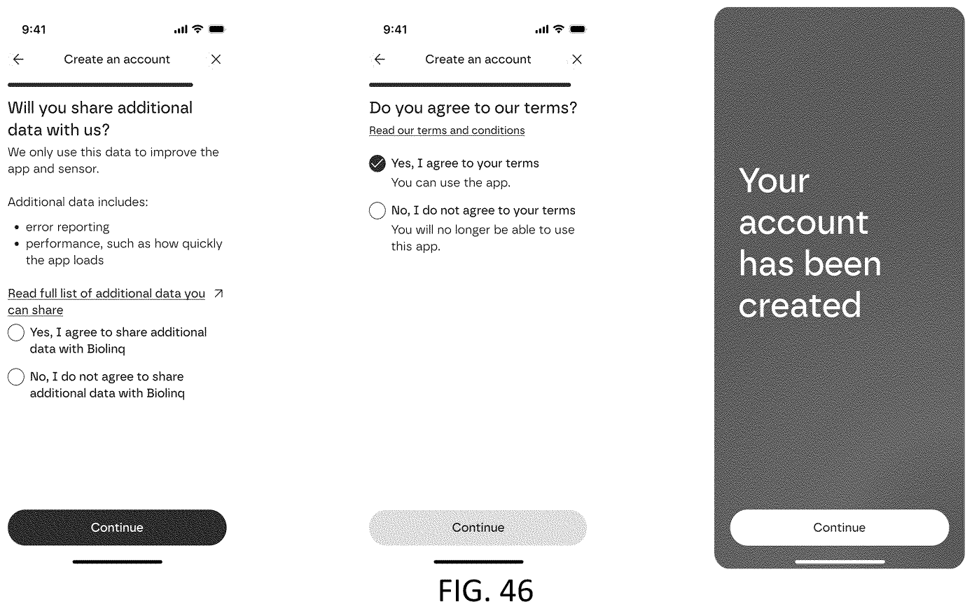

FIGS. 44, 45, and 46 provide a series of user registration user interfaces for creating a user account.

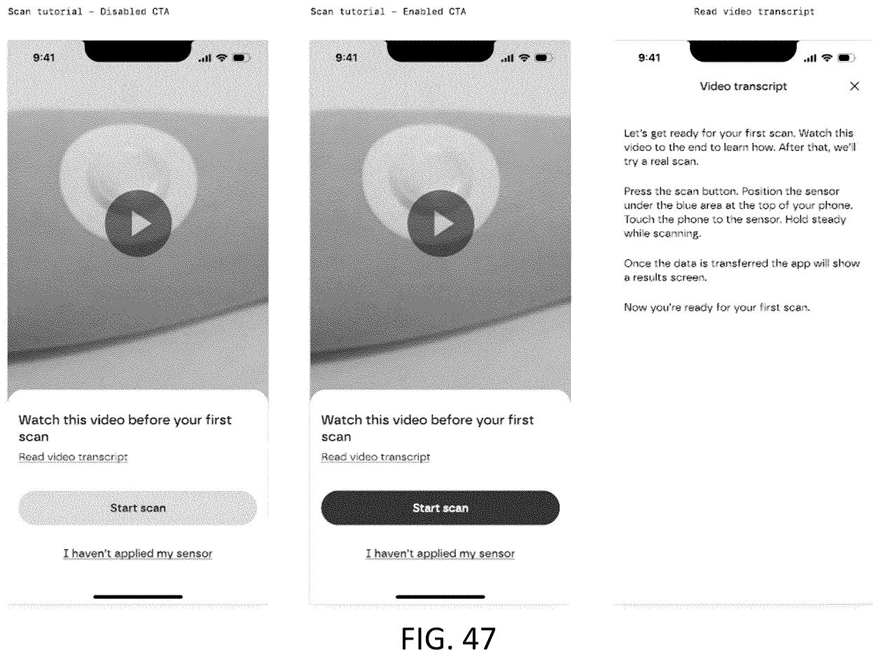

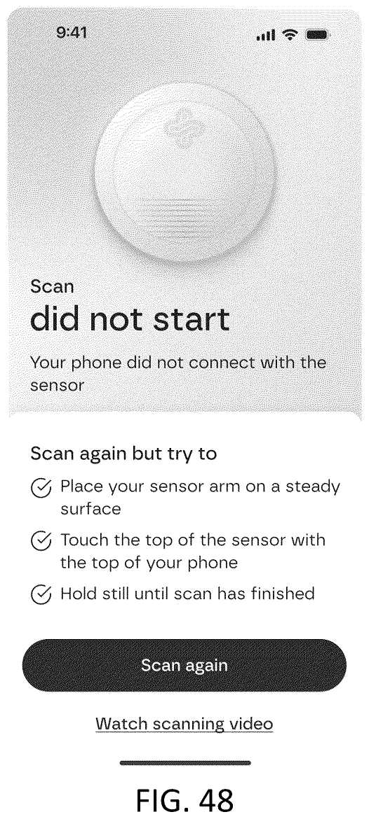

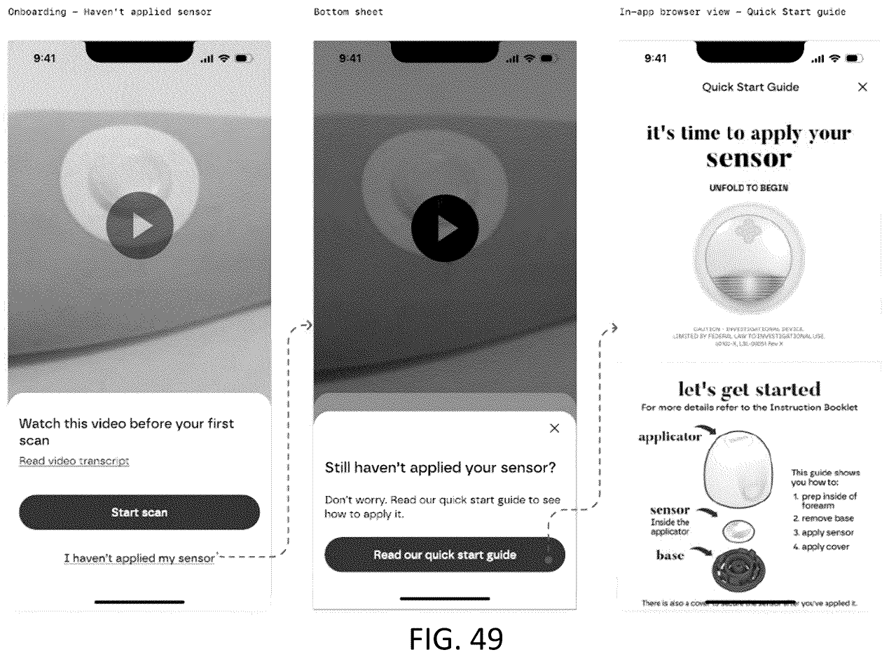

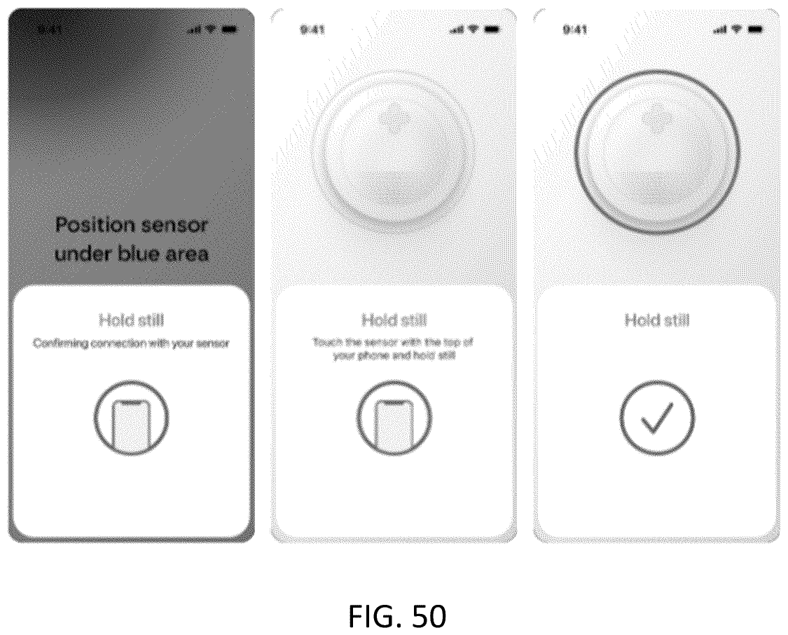

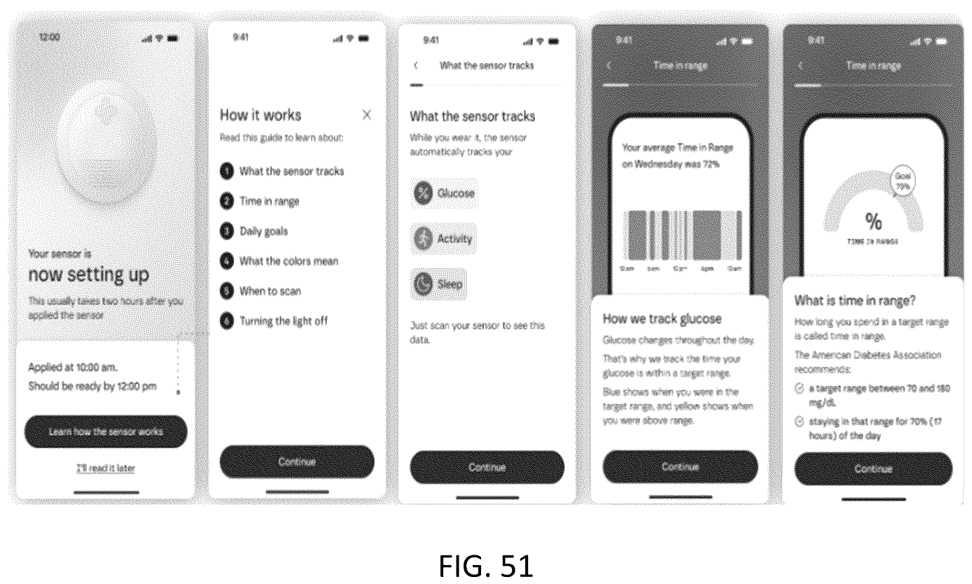

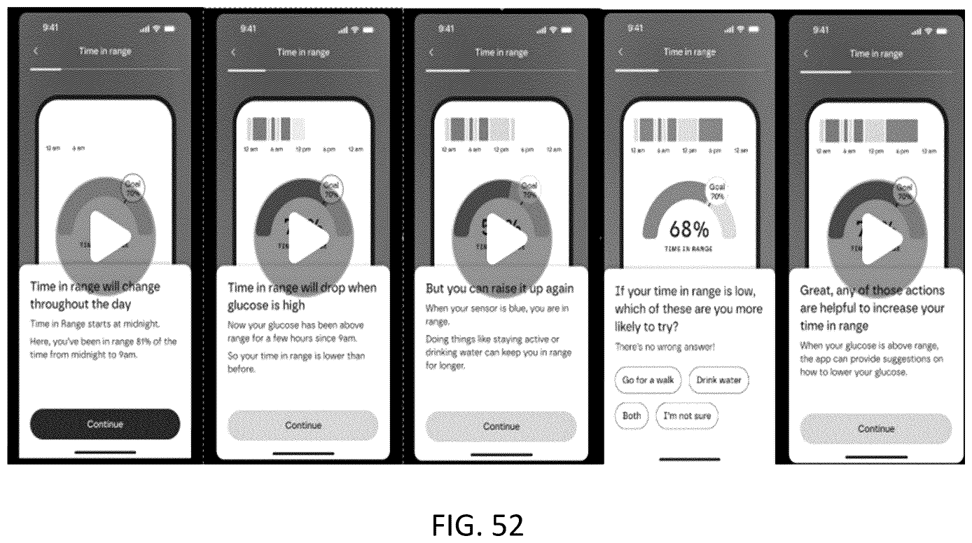

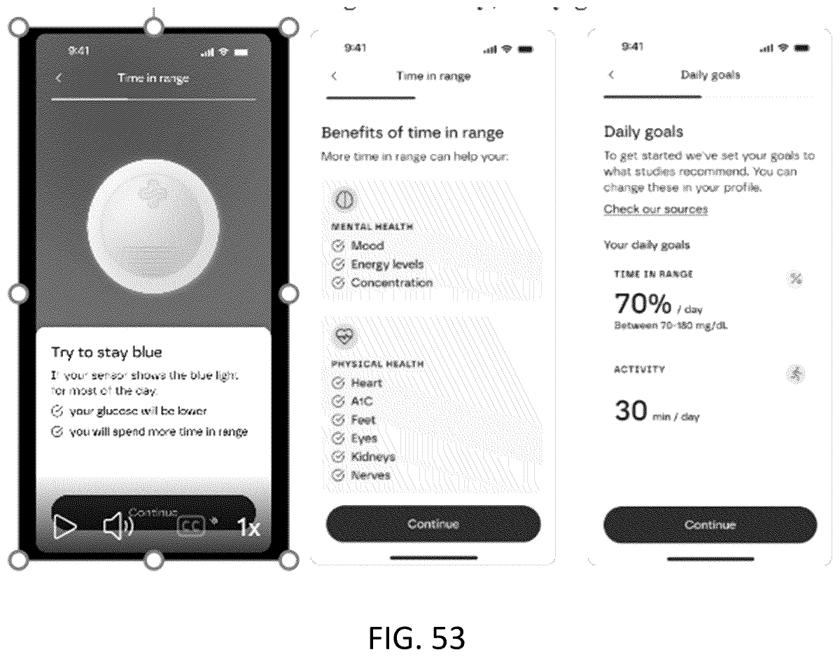

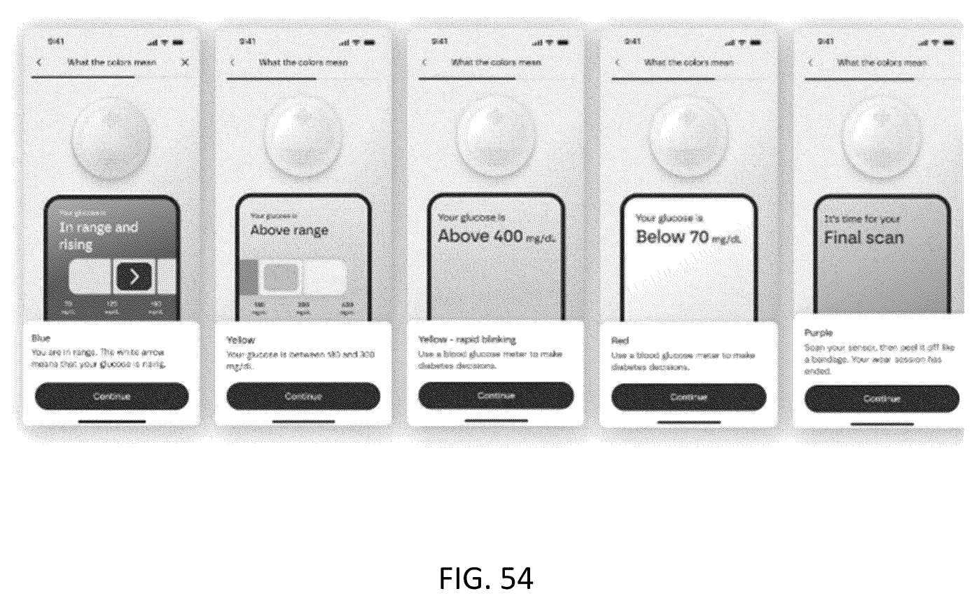

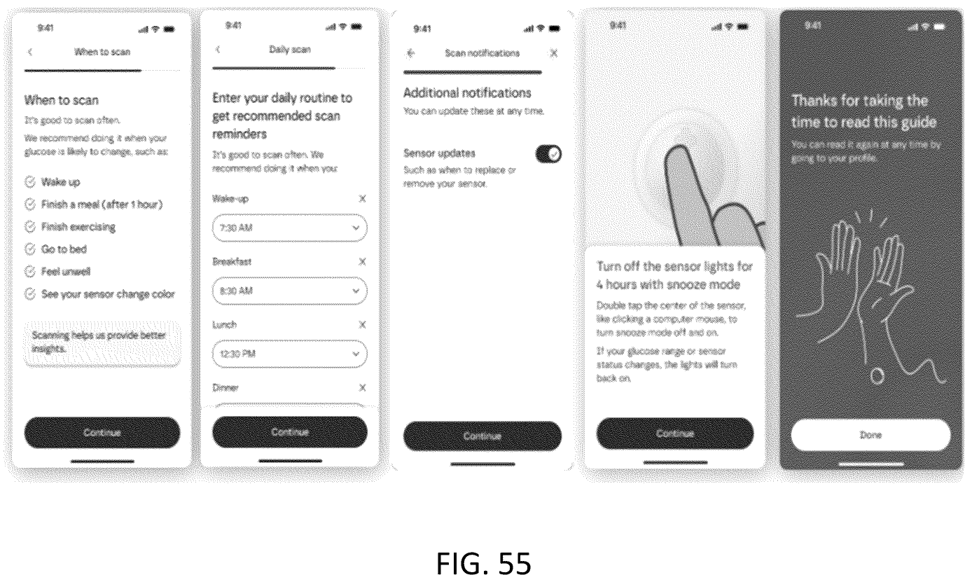

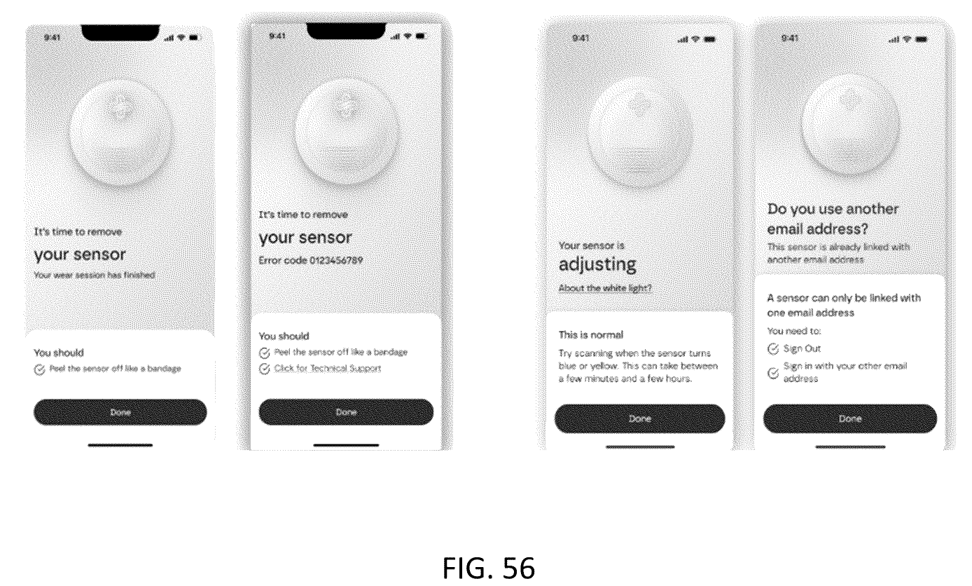

FIGS. 47-56 provide a series of onboarding user interfaces that guide the user through use and operation of the analyte monitoring device 110 and the associated mobile application.

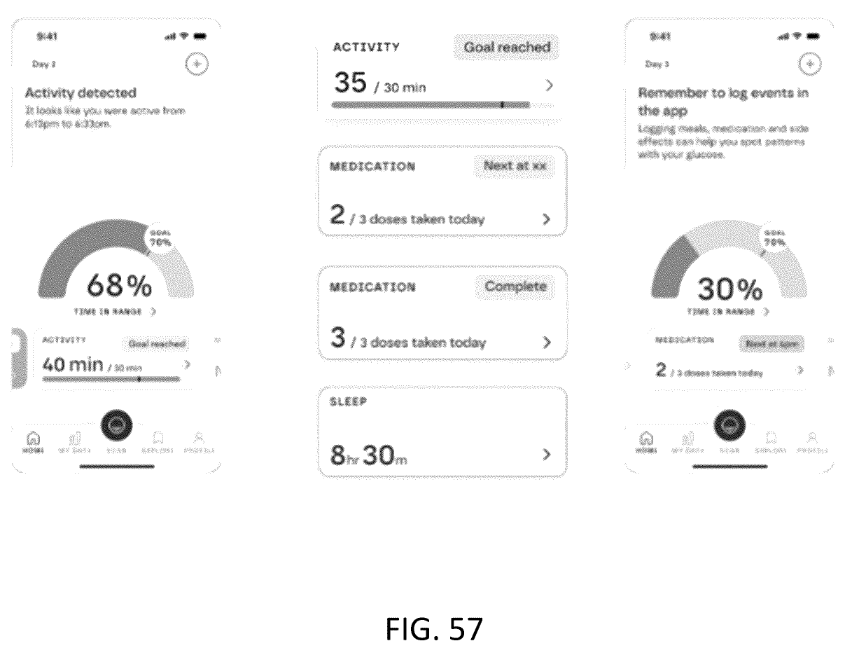

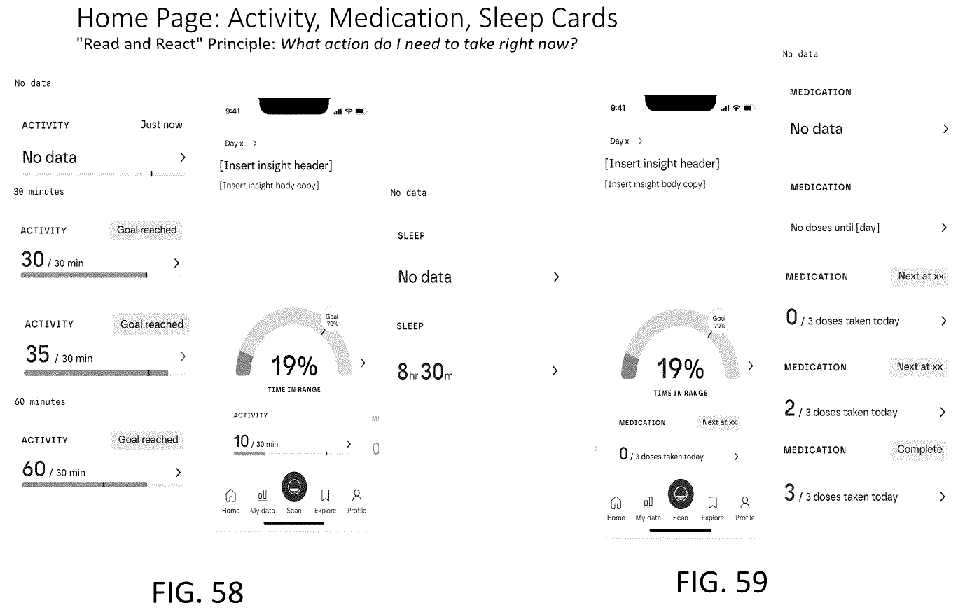

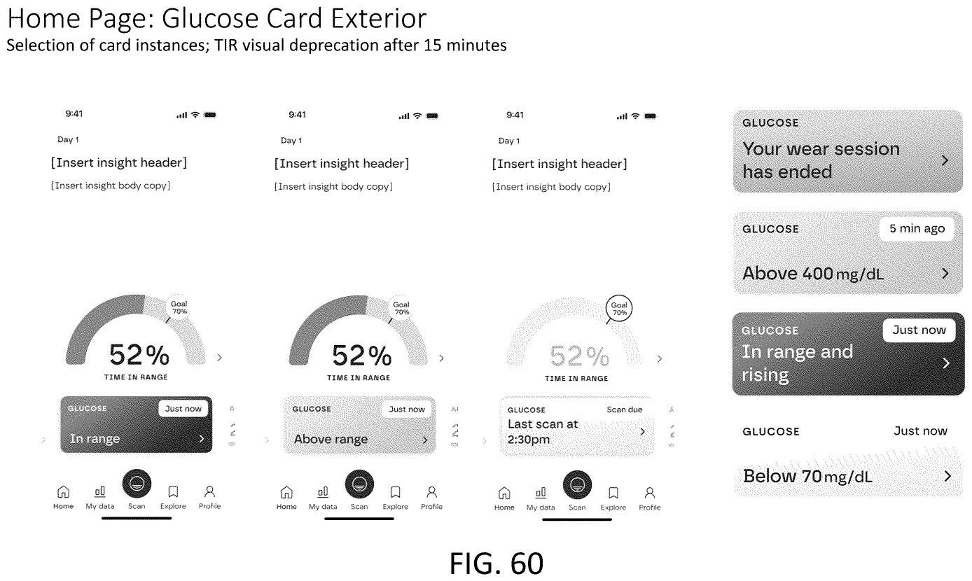

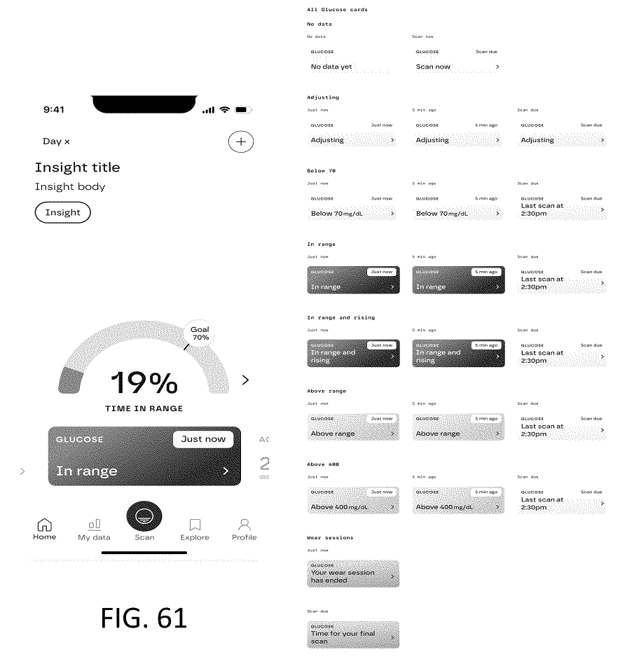

FIGS. 57-61 provide additional details related to the home user interface 2600.

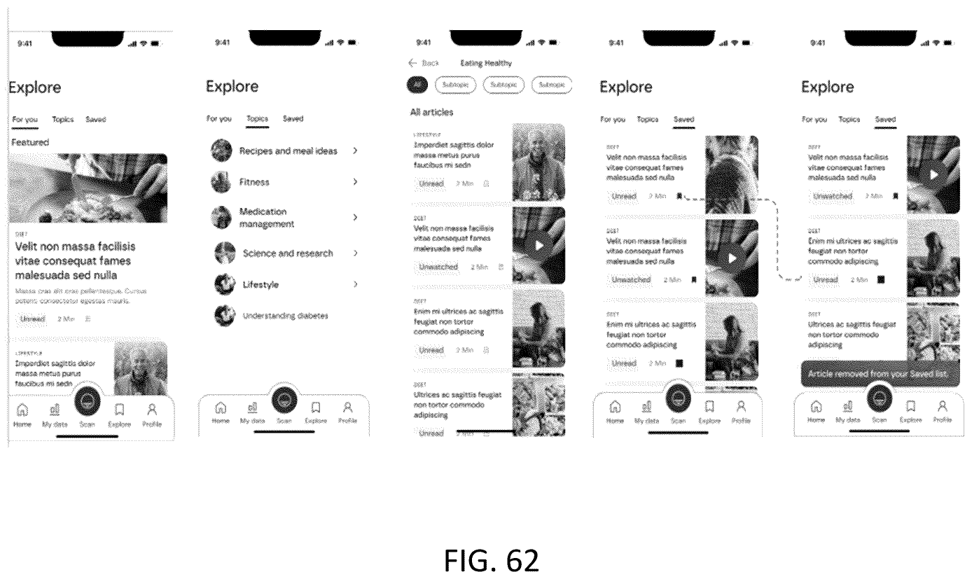

FIG. 62 depicts a series of explore user interfaces that provide the user with additional resources and information.

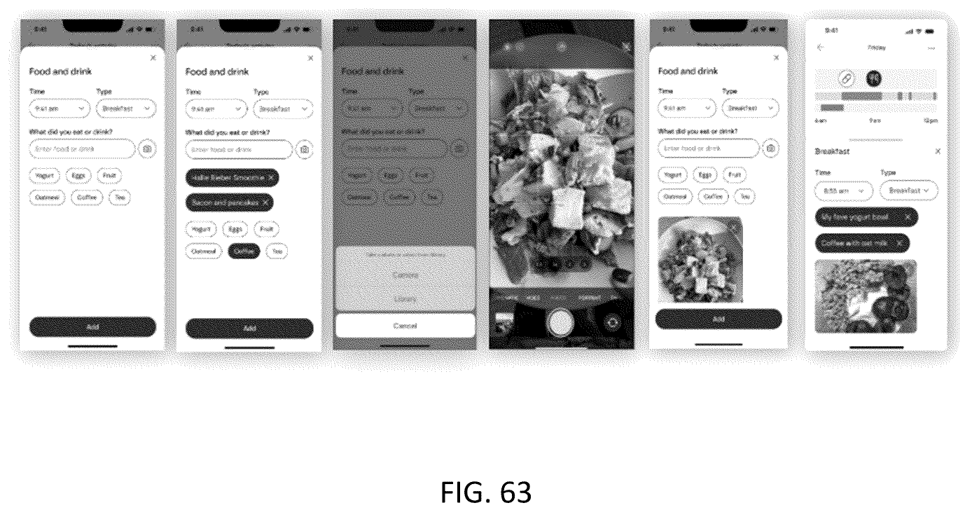

FIG. 63 depicts a series of additional food logging user interfaces for entering details related to meals and food eaten.

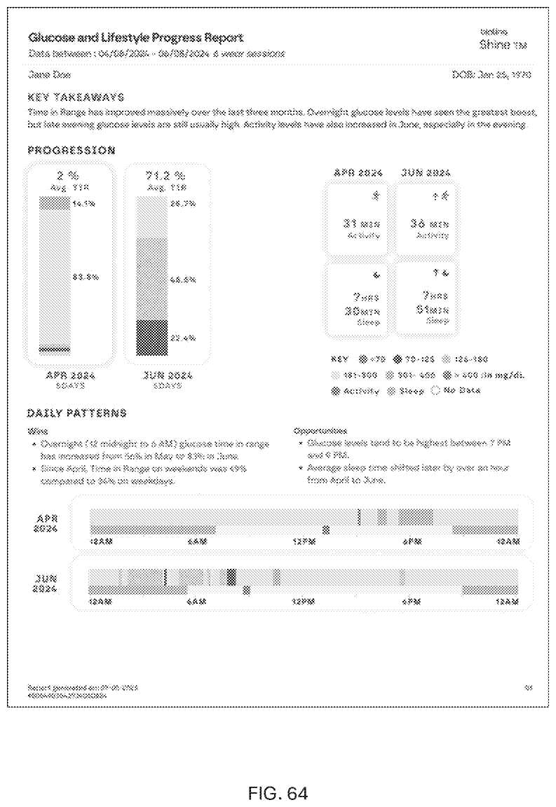

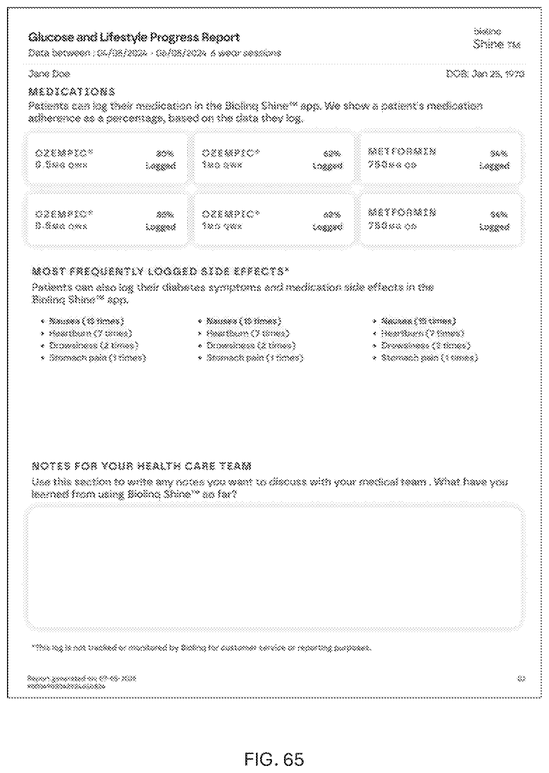

FIG. 64 and FIG. 65 depict an exemplary glucose and lifestyle progress report that may be generated by the mobile application. The glucose and lifestyle progress report may provide a summary of a user's analyte data, activity data, and sleep data over a plurality of wear sessions of the analyte monitoring device. In some variations, the glucose and lifestyle progress report may be generated for a predefined time period, such as data spanning multiple wear sessions over several months.

As shown in FIG. 64, the glucose and lifestyle progress report may include a key takeaways section that provides a summary of insights regarding the user's analyte levels, activity, and sleep patterns over the time period. The glucose and lifestyle progress report may further include a progression section that displays changes in the user's time in-range percentage across multiple wear sessions. In some variations, the progression section may include one or more pie charts or other graphical representations indicating a breakdown of time spent in various analyte ranges (e.g., below range, in-range, above range). The progression section may also include a comparison of activity and sleep data between wear sessions, such as average daily activity minutes and average sleep duration.

The glucose and lifestyle progress report may further include a daily patterns section that provides a time-aligned visualization of the user's analyte data, activity intervals, and sleep intervals across a 24-hour period. In some variations, the daily patterns section may display data from multiple wear sessions in separate rows to allow the user to compare patterns over time. The daily patterns section may include a key indicating color-coding for various analyte ranges (e.g., below 70 mg/dL, 70-125 mg/dL, 126-180 mg/dL, 181-300 mg/dL, 301-400 mg/dL, above 400 mg/dL), as well as color-coding for activity intervals and sleep intervals. The daily patterns section may further include a summary of wins and opportunities identified from the user's data, such as improvements in overnight glucose levels or identification of time periods with elevated glucose.

As shown in FIG. 65, the glucose and lifestyle progress report may include a medications section that displays medication adherence information based on data logged by the user in the mobile application. In some variations, the medications section may display one or more medication cards, each indicating a medication name, dosage, frequency, and/or a percentage representing logged adherence.

The glucose and lifestyle progress report may further include a most frequently logged side effects section that displays side effects logged by the user over the time period. In some variations, the side effects section may list side effects and a corresponding frequency of occurrence.

The glucose and lifestyle progress report may further include a notes section that provides a text entry field for the user to write notes for discussion with a healthcare team. In some variations, the notes section may not be tracked or monitored by the system for customer service or reporting purposes.

At the end of a wear session of the analyte monitoring device 110, the mobile computing device 102 may transfer the data related to the wear session to one or more servers or additional remote devices, which may generate wear reports such as those shown in FIG. 64 and FIG. 65. The wear reports summarize the wear session and highlight trends and patterns as well as other key takeaways.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

### ENUMERATED EMBODIMENTS

Embodiment 1: A method for detecting activity of a user, comprising: receiving activity data from a wearable sensor; determining an onset of an activity state based on (a) a first comparison of the received activity data to a first activity threshold over a first evaluation interval, and (b) a second comparison of the received activity data to a second, different activity threshold applied over a second, different evaluation interval, wherein the second evaluation interval precedes a portion of the first evaluation interval; and providing, via a display, activity information comprising a start time of the activity state based on the determined onset of the activity state.

Embodiment 2: The method of embodiment 1, wherein the activity data comprises a step count detected over a plurality of discrete sampling intervals.

Embodiment 3: The method of embodiment 2, wherein each of the plurality of discrete sampling intervals comprise about 5 minutes.

Embodiment 4: The method of embodiment 1, further comprising identifying a subset of the received activity data that meets a minimum activity threshold, wherein the received activity data for the determining is the subset of the received activity data.

Embodiment 5: The method of embodiment 1, wherein the second evaluation interval is shorter than the first evaluation interval.

Embodiment 6: The method of embodiment 1, wherein the first evaluation interval is about 10 minutes.

Embodiment 7: The method of embodiment 1, wherein the second evaluation interval is about 5 minutes.

Embodiment 8: The method of embodiment 1, wherein the first activity threshold is greater than the second activity threshold.

Embodiment 9: The method of embodiment 1, further comprising determining an end of the activity state based on a third, different activity threshold applied to the received activity data over a third, different evaluation interval.

Embodiment 10: The method of embodiment 9, wherein the determining the end of the activity state further comprises evaluating the received activity data over a fourth, different and subsequent evaluation interval.

Embodiment 11: The method of embodiment 9, wherein the activity information provided by the display further comprises an activity end time based on the determined end of the activity state.

Embodiment 12: The method of embodiment 1, further comprising, prior to determining the onset of the activity state, inserting an inactivity value correlated with any gaps in the activity data exceeding a threshold gap duration.

Embodiment 13: The method of embodiment 12, wherein the threshold gap duration comprises about 5 to about 10 minutes.

Embodiment 14: The method of embodiment 1, wherein the first and second activity thresholds are configured to reduce false positive activity detections.

Embodiment 15: The method of embodiment 1, wherein a remote device receives the activity data.

Embodiment 16: The method of embodiment 1, further comprising aggregating the activity data received over a plurality of discrete sampling intervals.

Embodiment 17: The method of embodiment 16, further comprising classifying the aggregated activity data as one or more of a low activity state, a moderate activity state, or a high activity state over a cumulative time duration comprising the plurality of discrete sampling intervals.

Embodiment 18: The method of embodiment 1, wherein providing the activity information further comprises displaying a time-based visualization including one or more segments aligned along a common timeline, the one or more segments representing one or more activity states.

Embodiment 19: The method of embodiment 18, wherein the detected activity state is displayed as a visually differentiated segment extending between the start time and an end time along the timeline.

Embodiment 20: The method of embodiment 18, wherein the timeline simultaneously displays the activity information together with one or more of sleep information, medication information, or glucose information.

Embodiment 21: The method of embodiment 18, further comprising receiving user input to selectively display or hide one or more categories of information on the timeline.

Embodiment 22: The method of embodiment 18, further comprising receiving user input selecting a temporal span of the timeline.

Embodiment 23: The method of embodiment 1, wherein the activity information displayed comprises the start time and a duration of the detected activity state.

Embodiment 24: The method of embodiment 23, further comprising classifying the activity state as an activity type, and displaying the activity type.

Embodiment 25: The method of embodiment 24, further comprising receiving user input modifying at least one of the start time, duration, or activity type associated with the activity state.

Embodiment 26: A system for detecting activity of a user, comprising: a wearable sensor; and a remote device comprising a display and communicably coupled with the wearable sensor, wherein the remote device is configured to: receive activity data from the wearable sensor; determine an onset of an activity state based on (a) a first comparison of the received activity data to a first activity threshold over a first evaluation interval, and (b) a second comparison of the received activity data to a second, different activity threshold applied over a second, different evaluation interval, wherein the second evaluation interval precedes a portion of the first evaluation interval; and provide, via the display, activity information comprising a start time of the activity state based on the determined onset of the activity state.

Embodiment 27: A non-transitory computer-readable medium storing instructions that, when executed by one or more processors, cause the processors to: receive activity data from a wearable sensor; determine an onset of an activity state based on (a) a first comparison of the received activity data to a first activity threshold over a first evaluation interval, and (b) a second comparison of the received activity data to a second, different activity threshold applied over a second, different evaluation interval, wherein the second evaluation interval precedes a portion of the first evaluation interval; and provide, via a display, activity information comprising a start time of the activity state based on the determined onset of the activity state.

Embodiment 28: A method for detecting inactivity of a user, comprising: receiving activity data from a first wearable device; receiving light data from the first wearable device or a second wearable device; determining a user-specific sleep reference time; transforming a current time to a normalized time based on the user-specific sleep reference time; generating an inactivity probability based on the activity data, the light data, and the normalized time; identifying an inactivity interval when the inactivity probability exceeds a threshold for at least a minimum evaluation interval; and providing, via a display, inactivity information comprising the identified inactivity interval.

Embodiment 29: The method of embodiment 28, wherein determining the user-specific sleep reference time comprises receiving user input indicative of a typical sleep time or typical wake time.

Embodiment 30: The method of embodiment 28, wherein transforming the current time comprises shifting the current time relative to the user-specific sleep reference time to constrain the normalized time within a bounded range.

Embodiment 31: The method of embodiment 30, wherein the bounded range comprises about −12 hours to about +12 hours relative to the user-specific sleep reference time.

Embodiment 32: The method of embodiment 28, wherein a predictive model receives the activity data, the light data, and the normalized time as input to generate the inactivity probability.

Embodiment 33: The method of embodiment 32, wherein the predictive model further receives as an input a proximity to the user-specific sleep reference time.

Embodiment 34: The method of embodiment 32, wherein the predictive model comprises a layered neural network.

Embodiment 35: The method of embodiment 28, wherein the inactivity probability is generated using the activity data and the light data from time intervals before and after the current time.

Embodiment 36: The method of embodiment 28, wherein the minimum evaluation interval comprises a plurality of consecutive evaluation intervals, and wherein the inactivity interval is identified when the inactivity probability exceeds the threshold over the plurality of consecutive evaluation intervals.

Embodiment 37: The method of embodiment 36, wherein each of the plurality of consecutive evaluation intervals corresponds to at least about one hour.

Embodiment 38: The method of embodiment 28, wherein the activity data and the light data are received in discrete sampling intervals of about 5 minutes.

Embodiment 39: The method of embodiment 28, further comprising discretizing the light data into light level values.

Embodiment 40: The method of embodiment 39, wherein the light level values comprise values of 1 to 5.

Embodiment 41: A system for detecting inactivity of a user, the system comprising: a first wearable device; and a remote device comprising a display and communicably coupled to the first wearable device, wherein the remote device is configured to: receive activity data from the first wearable device; receive light data from the first wearable device or a second wearable device; determine a user-specific sleep reference time; transform a current time to a normalized time based on the user-specific sleep reference time; generate an inactivity probability based on the activity data, the light data, and the normalized time; identify an inactivity interval when the inactivity probability exceeds a threshold for at least a minimum evaluation interval; and provide, via the display, inactivity information comprising the identified inactivity interval.

Embodiment 42: A non-transitory computer-readable medium storing instructions that, when executed by one or more processors, cause the processors to: receive activity data from a first wearable device; receive light data from the first wearable device or a second wearable device; determine a user-specific sleep reference time; transform a current time to a normalized time based on the user-specific sleep reference time; generate an inactivity probability based on the activity data, the light data, and the normalized time; identify an inactivity interval when the inactivity probability exceeds a threshold for at least a minimum evaluation interval; and provide, via display, inactivity information comprising the identified inactivity interval.

Embodiment 43: A method for determining an inactivity of a user, comprising: receiving activity data from a first wearable device; receiving light data from the first wearable device or a second wearable device; aggregating the activity data and the light data within a first evaluation interval preceding a target time and a second evaluation interval following the target time to define a characterization interval centered on the target time; quantizing the light data within the characterization interval; determining, based on the activity data and the quantized light data within the characterization interval, an inactivity probability associated with the target time; and assigning an inactivity interval to the target time when the inactivity probability exceeds a threshold for at least a minimum evaluation interval.

Embodiment 44: The method of embodiment 43, wherein each of the first evaluation interval and the second evaluation interval comprises about one hour.

Embodiment 45: The method of embodiment 43, wherein quantizing the light data comprises assigning light level values of 1 to 5 to measurements within the light data.

Embodiment 46: The method of embodiment 45, wherein the activity data comprises a step count, and wherein determining the inactivity probability comprises correlating a reduction in the step count with a reduction in light level values within the characterization interval.

Embodiment 47: The method of embodiment 43, wherein the minimum evaluation interval comprises a continuous duration of about 60 minutes.

Embodiment 48: The method of embodiment 43, wherein the inactivity state comprises a sleep state.

Embodiment 49: The method of embodiment 43, wherein an awake state is assigned when the inactivity probability exceeds the threshold but does not persist for the minimum evaluation interval.

Embodiment 50: A system for detecting inactivity of a user, the system comprising: a first wearable device; and a remote device comprising a display and communicably coupled to the first wearable device, wherein the remote device is configured to: receive activity data from the first wearable device; receive light data from the first wearable device or a second wearable device; aggregate the activity data and the light data within a first evaluation interval preceding a target time and a second evaluation interval following the target time to define a characterization interval centered on the target time; quantize the light data within the characterization interval; determine, based on the activity data and the quantized light data within the characterization interval, an inactivity probability associated with the target time; and assign an inactivity interval to the target time when the inactivity probability exceeds a threshold for at least a minimum evaluation interval.

Embodiment 51: A method for graphically representing physiological state information of a user, comprising: receiving, at a processor, physiological data comprising activity intervals, sleep intervals, and analyte monitoring data for the user; providing the activity intervals, the sleep intervals, and the analyte monitoring data via a graphical user interface (GUI), wherein the GUI comprises: a timeline extending across a temporal span; a first segment aligned in a first row along the timeline and representing analyte monitoring data; a second segment aligned in a second row along the timeline and representing a first activity interval of the received activity intervals; and a third segment aligned in the second row along the timeline and representing a first sleep interval of the received sleep intervals, wherein the first segment represents continuous analyte monitoring data over a first portion of the timeline, and the second and third segments represent discrete, noncontinuous traces of activity and sleep, respectively, that are separated by a temporal gap over the first portion of the timeline; and one or more user interface controls configured to selectively display or hide one or more categories of physiological information comprising at least one of the analyte monitoring data, the activity intervals, or the sleep intervals.

Embodiment 52: The method of embodiment 51, wherein at least one of the first activity interval or the first inactivity interval is displayed with a start time and a duration.

Embodiment 53: The method of embodiment 52, further comprising receiving user input modifying at least one of a start time or duration associated with the first activity interval or the first inactivity interval.

Embodiment 54: The method of embodiment 51, wherein the GUI is configured to re-render the second row with at least one of a modified second segment or a modified third segment without re-rendering the first row.

Embodiment 55: The method of embodiment 51, wherein the analyte monitoring data comprises glucose information.

Embodiment 56: The method of embodiment 51, wherein the first segment comprises at least one of a first subsegment that represents in-range analyte monitoring data or a second subsegment that represents out-of-range analyte monitoring data.

Embodiment 57: The method of embodiment 54, wherein the first segment comprises the first and second subsegments, and wherein no temporal gaps are provided between the first and second subsegments.

Embodiment 58: The method of embodiment 51, wherein the one or more categories of physiological information further comprise medication data.

Embodiment 59: The method of embodiment 51, wherein the temporal span is selectable, and the selectable temporal span comprises one or more of a 12-hour view, a 24-hour view, or a multi-day view.

Embodiment 60: The method of embodiment 51, wherein the first, second, and third segments each comprise a unique color.

Embodiment 61: A wearable analyte monitoring device, comprising: an ambient light sensor configured to sense a level of ambient light; a kinetic sensor configured to monitor a step count of a user; a clock configured to monitor a time; and a microcontroller comprising a trained machine learning model configured to determine a state of rest of the user based on input signals from the ambient light sensor, the kinetic sensor, and the clock.

Embodiment 62: The wearable analyte monitoring device of embodiment 61, wherein the trained machine learning model further utilizes time parameters inputted by the user to determine the state of rest.

Embodiment 63: The wearable analyte monitoring device of embodiment 61, further comprising a microneedle array comprising a plurality of microneedles configured to detect an analyte.

Embodiment 64: The wearable analyte monitoring device of embodiment 61, further comprising a communication controller configured to transmit state of rest data to a mobile device, wherein the mobile device is configured to execute a mobile application to display one or more user interfaces including representations of the state of rest data.

1.-27. (canceled)

28. A method for detecting inactivity of a user, comprising:
receiving activity data from a first wearable device;
receiving light data from the first wearable device or a second wearable device;
determining a user-specific sleep reference time;
transforming a current time to a normalized time based on the user-specific sleep reference time;
generating an inactivity probability based on the activity data, the light data, and the normalized time;
identifying an inactivity interval when the inactivity probability exceeds a threshold for at least a minimum evaluation interval; and
providing, via a display, inactivity information comprising the identified inactivity interval.

29. The method of claim 28, wherein determining the user-specific sleep reference time comprises receiving user input indicative of a typical sleep time or typical wake time.

30. The method of claim 28, wherein transforming the current time comprises shifting the current time relative to the user-specific sleep reference time to constrain the normalized time within a bounded range.

31. The method of claim 30, wherein the bounded range comprises about −12 hours to about +12 hours relative to the user-specific sleep reference time.

32. The method of claim 28, wherein a predictive model receives the activity data, the light data, and the normalized time as input to generate the inactivity probability.

33. The method of claim 32, wherein the predictive model further receives as an input a proximity to the user-specific sleep reference time.

34. The method of claim 32, wherein the predictive model comprises a layered neural network.

35. The method of claim 28, wherein the inactivity probability is generated using the activity data and the light data from time intervals before and after the current time.

36. The method of claim 28, wherein the minimum evaluation interval comprises a plurality of consecutive evaluation intervals, and wherein the inactivity interval is identified when the inactivity probability exceeds the threshold over the plurality of consecutive evaluation intervals.

37. The method of claim 36, wherein each of the plurality of consecutive evaluation intervals corresponds to at least about one hour.

38. The method of claim 28, wherein the activity data and the light data are received in discrete sampling intervals of about 5 minutes.

39. The method of claim 28, further comprising discretizing the light data into light level values.

40. The method of claim 39, wherein the light level values comprise values of 1 to 5.

41. A system for detecting inactivity of a user, the system comprising:
a first wearable device; and
a remote device comprising a display and communicably coupled to the first wearable device, wherein the remote device is configured to:
receive activity data from the first wearable device;
receive light data from the first wearable device or a second wearable device;
determine a user-specific sleep reference time;
transform a current time to a normalized time based on the user-specific sleep reference time;
generate an inactivity probability based on the activity data, the light data, and the normalized time;
identify an inactivity interval when the inactivity probability exceeds a threshold for at least a minimum evaluation interval; and
provide, via the display, inactivity information comprising the identified inactivity interval.

42. A non-transitory computer-readable medium storing instructions that, when executed by one or more processors, cause the processors to:
receive activity data from a first wearable device;
receive light data from the first wearable device or a second wearable device;
determine a user-specific sleep reference time;
transform a current time to a normalized time based on the user-specific sleep reference time;
generate an inactivity probability based on the activity data, the light data, and the normalized time;
identify an inactivity interval when the inactivity probability exceeds a threshold for at least a minimum evaluation interval; and
provide, via display, inactivity information comprising the identified inactivity interval.

43.-64. (canceled)
